SEQUENCE LISTING (290 sequences)

SEQ ID NO: 1 - DNA, 20 bp, Artificial sequence
  sgRNA
gggcagtgtttcaaaatcca

SEQ ID NO: 2 - DNA, 20 bp, Artificial sequence
  sgRNA
ggaacacgagccgtcagcgg

SEQ ID NO: 3 - DNA, 20 bp, Artificial sequence
  sgRNA
ggatttctgttctgcatcaa

SEQ ID NO: 4 - DNA, 20 bp, Artificial sequence
  sgRNA
ggtcccatctgttgcctcca

SEQ ID NO: 5 - DNA, 20 bp, Artificial sequence
  sgRNA
gtttggagatccactcgata

SEQ ID NO: 6 - DNA, 20 bp, Artificial sequence
  sgRNA
gtacccagtgcaagctacag

SEQ ID NO: 7 - DNA, 20 bp, Artificial sequence
  sgRNA
gggtaccccgctatatgttg

SEQ ID NO: 8 - DNA, 20 bp, Artificial sequence
  sgRNA
gcatctggcccccaggagat

SEQ ID NO: 9 - DNA, 20 bp, Artificial sequence
  sgRNA
ggtccgtactgagacatctg

SEQ ID NO: 10 - DNA, 20 bp, Artificial sequence
  sgRNA
gattctgactggcttccagg

SEQ ID NO: 11 - DNA, 20 bp, Artificial sequence
  sgRNA
gtgaaatactctccatcaag

SEQ ID NO: 12 - DNA, 20 bp, Artificial sequence
  sgRNA
gggagaggcgcttgtgcagg

SEQ ID NO: 13 - DNA, 20 bp, Artificial sequence
  sgRNA
gcaaagagaggtacgcaggc

SEQ ID NO: 14 - DNA, 20 bp, Artificial sequence
  sgRNA
gcggttcatgccccccatgc

SEQ ID NO: 15 - DNA, 20 bp, Artificial sequence
  sgRNA
ggtatactcagagccggcct

SEQ ID NO: 16 - DNA, 20 bp, Artificial sequence
  sgRNA
gccatggctgaaggggagcc

SEQ ID NO: 17 - DNA, 20 bp, Artificial sequence
  sgRNA
gactctgtaggtgacagcaa

SEQ ID NO: 18 - DNA, 20 bp, Artificial sequence
  sgRNA
gctcctaaacctacttgttc

SEQ ID NO: 19 - DNA, 20 bp, Artificial sequence
  sgRNA
gcatgctgtatggtaccagg

SEQ ID NO: 20 - DNA, 20 bp, Artificial sequence
  sgRNA
gaaggcatctaccgggtcag

SEQ ID NO: 21 - DNA, 20 bp, Artificial sequence
  sgRNA
ggacgatgatgaggacgatg

SEQ ID NO: 22 - DNA, 20 bp, Artificial sequence
  sgRNA
gtgtaatttcaaagccccct

SEQ ID NO: 23 - DNA, 20 bp, Artificial sequence
  sgRNA
gtggacagcatctagtaggt

SEQ ID NO: 24 - DNA, 20 bp, Artificial sequence
  sgRNA
gggcggttctcttcccaaag

SEQ ID NO: 25 - DNA, 20 bp, Artificial sequence
  sgRNA
ggaagagaaccgccctattg

SEQ ID NO: 26 - DNA, 20 bp, Artificial sequence
  sgRNA
gcatatgctcgtaaagtgga

SEQ ID NO: 27 - DNA, 20 bp, Artificial sequence
  sgRNA
gatgagtgaaaatgctggtg

SEQ ID NO: 28 - DNA, 20 bp, Artificial sequence
  sgRNA
ggctgagctgctgttggcaa

SEQ ID NO: 29 - DNA, 20 bp, Artificial sequence
  sgRNA
gccctaggtgctagtcctat

SEQ ID NO: 30 - DNA, 20 bp, Artificial sequence
  sgRNA
gctagtcctatgggtgtaaa

SEQ ID NO: 31 - DNA, 20 bp, Artificial sequence
  sgRNA
gccggctatgtcgggcctgt

SEQ ID NO: 32 - DNA, 20 bp, Artificial sequence
  sgRNA
gggattcagttctgctgagc

SEQ ID NO: 33 - DNA, 20 bp, Artificial sequence
  sgRNA
gtgtgtgagacatgtgacaa

SEQ ID NO: 34 - DNA, 20 bp, Artificial sequence
  sgRNA
gcaggcaggtcctccatagg

SEQ ID NO: 35 - DNA, 20 bp, Artificial sequence
  sgRNA
gcctcctctgccggagaaag

SEQ ID NO: 36 - DNA, 20 bp, Artificial sequence
  sgRNA
gactgtgaatggacagctga

SEQ ID NO: 37 - DNA, 20 bp, Artificial sequence
  sgRNA
gtcttctcaaaacatttcag

SEQ ID NO: 38 - DNA, 20 bp, Artificial sequence
  sgRNA
gcactgatggaaaatggtga

SEQ ID NO: 39 - DNA, 20 bp, Artificial sequence
  sgRNA
gactaccagttccaaagata

SEQ ID NO: 40 - DNA, 20 bp, Artificial sequence
  sgRNA
gaagcttctggttactttcc

SEQ ID NO: 41 - DNA, 20 bp, Artificial sequence
  sgRNA
gggattggccgcgaagttcc

SEQ ID NO: 42 - DNA, 20 bp, Artificial sequence
  sgRNA
ggggtacgaccgaaagagtt

SEQ ID NO: 43 - DNA, 20 bp, Artificial sequence
  sgRNA
gggtcgcctgccgctcgact

SEQ ID NO: 44 - DNA, 20 bp, Artificial sequence
  sgRNA
gggaacgtcgcccagaccga

SEQ ID NO: 45 - DNA, 20 bp, Artificial sequence
  sgRNA
ggggtacgaccgaaagagtt

SEQ ID NO: 46 - DNA, 20 bp, Artificial sequence
  sgRNA
gtacctgcaccaggaaaacc

SEQ ID NO: 47 - DNA, 20 bp, Artificial sequence
  sgRNA
gtggagccatacattgcatg

SEQ ID NO: 48 - DNA, 20 bp, Artificial sequence
  sgRNA
gggtgagttttctgtctagt

SEQ ID NO: 49 - DNA, 20 bp, Artificial sequence
  sgRNA
gcctttgtcatcagaattcg

SEQ ID NO: 50 - DNA, 20 bp, Artificial sequence
  sgRNA
gaaggcaaagcactatcaca

SEQ ID NO: 51 - DNA, 20 bp, Artificial sequence
  sgRNA
gcaatggtcttgagatctat

SEQ ID NO: 52 - DNA, 20 bp, Artificial sequence
  sgRNA
gaccattgctcagaggatac

SEQ ID NO: 53 - DNA, 20 bp, Artificial sequence
  sgRNA
gcctgggtctcaagtattca

SEQ ID NO: 54 - DNA, 20 bp, Artificial sequence
  sgRNA
gccaaaacatacaatgagcc

SEQ ID NO: 55 - DNA, 20 bp, Artificial sequence
  sgRNA
gtgcgaaggacctgtcagcc

SEQ ID NO: 56 - DNA, 20 bp, Artificial sequence
  sgRNA
gactgcatgggcagaaggga

SEQ ID NO: 57 - DNA, 20 bp, Artificial sequence
  sgRNA
gagacggcactttccttgtc

SEQ ID NO: 58 - DNA, 20 bp, Artificial sequence
  sgRNA
gttggctacagtagtgggct

SEQ ID NO: 59 - DNA, 20 bp, Artificial sequence
  sgRNA
ggcagtgctgcaggcaaaag

SEQ ID NO: 60 - DNA, 20 bp, Artificial sequence
  sgRNA
ggctgacgcagaaaggtgtg

SEQ ID NO: 61 - DNA, 20 bp, Artificial sequence
  sgRNA
ggccgcaagctgacgcctca

SEQ ID NO: 62 - DNA, 20 bp, Artificial sequence
  sgRNA
gcctcagggacagagagacc

SEQ ID NO: 63 - DNA, 20 bp, Artificial sequence
  sgRNA
gtccctgaggcgtcagcttg

SEQ ID NO: 64 - DNA, 20 bp, Artificial sequence
  sgRNA
gggccgcaagctgacgcctc

SEQ ID NO: 65 - DNA, 20 bp, Artificial sequence
  sgRNA
gttgaaacagagcggggggg

SEQ ID NO: 66 - DNA, 20 bp, Artificial sequence
  sgRNA
gtggatgaaaggctcttcat

SEQ ID NO: 67 - DNA, 20 bp, Artificial sequence
  sgRNA
ggttttgcacagtctcttcc

SEQ ID NO: 68 - DNA, 20 bp, Artificial sequence
  sgRNA
gacctcaggctgaacagcct

SEQ ID NO: 69 - DNA, 20 bp, Artificial sequence
  sgRNA
ggcccaggctgttcagcctg

SEQ ID NO: 70 - DNA, 20 bp, Artificial sequence
  sgRNA
gtccaccaccccctggtcac

SEQ ID NO: 71 - DNA, 20 bp, Artificial sequence
  sgRNA
gttggcactgatttcataac

SEQ ID NO: 72 - DNA, 20 bp, Artificial sequence
  sgRNA
gggagaagatagcaagatgc

SEQ ID NO: 73 - DNA, 20 bp, Artificial sequence
  sgRNA
gtggctactgaccaaaccca

SEQ ID NO: 74 - DNA, 20 bp, Artificial sequence
  sgRNA
gagaattcctaacagctatg

SEQ ID NO: 75 - DNA, 20 bp, Artificial sequence
  sgRNA
gctgccgatactccaaactt

SEQ ID NO: 76 - DNA, 20 bp, Artificial sequence
  sgRNA
gcaactattttacaacaatt

SEQ ID NO: 77 - DNA, 20 bp, Artificial sequence
  sgRNA
ggtaaattaaaacactcacc

SEQ ID NO: 78 - DNA, 20 bp, Artificial sequence
  sgRNA
gtaaattaaaacactcacct

SEQ ID NO: 79 - DNA, 20 bp, Artificial sequence
  sgRNA
gcagcattttcagttagctt

SEQ ID NO: 80 - DNA, 20 bp, Artificial sequence
  sgRNA
ggctattaaagcatttcagg

SEQ ID NO: 81 - DNA, 20 bp, Artificial sequence
  sgRNA
gtgattttgatctcgtgcct

SEQ ID NO: 82 - DNA, 20 bp, Artificial sequence
  sgRNA
gcaaggtacactgtaatcag

SEQ ID NO: 83 - DNA, 20 bp, Artificial sequence
  sgRNA
gtgcttatgaatccatgaaa

SEQ ID NO: 84 - DNA, 20 bp, Artificial sequence
  sgRNA
gtccaaatatatagtaaggt

SEQ ID NO: 85 - DNA, 20 bp, Artificial sequence
  sgRNA
gagacttgaggaaaatgtta

SEQ ID NO: 86 - DNA, 20 bp, Artificial sequence
  sgRNA
ggggccaagggtatgccaga

SEQ ID NO: 87 - DNA, 20 bp, Artificial sequence
  sgRNA
gactgtgggatcccagtttc

SEQ ID NO: 88 - DNA, 20 bp, Artificial sequence
  sgRNA
gtaggtaggaggtgaactca

SEQ ID NO: 89 - DNA, 20 bp, Artificial sequence
  sgRNA
gcatgttcaacatcgtaggt

SEQ ID NO: 90 - DNA, 20 bp, Artificial sequence
  sgRNA
ggagtcttctgcctggttcc

SEQ ID NO: 91 - DNA, 20 bp, Artificial sequence
  sgRNA
ggactacccaagtgtgcgga

SEQ ID NO: 92 - DNA, 20 bp, Artificial sequence
  sgRNA
gcaccttgagagtcagcacc

SEQ ID NO: 93 - DNA, 20 bp, Artificial sequence
  sgRNA
ggttaaccagaagtccatca

SEQ ID NO: 94 - DNA, 20 bp, Artificial sequence
  sgRNA
gtgccatccctcaatgtcga

SEQ ID NO: 95 - DNA, 20 bp, Artificial sequence
  sgRNA
gtcctgaggagatggaggct

SEQ ID NO: 96 - DNA, 20 bp, Artificial sequence
  sgRNA
gtctccttcttctagaggca

SEQ ID NO: 97 - DNA, 20 bp, Artificial sequence
  sgRNA
ggcaaaacagaataggagag

SEQ ID NO: 98 - DNA, 20 bp, Artificial sequence
  sgRNA
ggcaatttgatacaagtaac

SEQ ID NO: 99 - DNA, 20 bp, Artificial sequence
  sgRNA
gcaatttgatacaagtaact

SEQ ID NO: 100 - DNA, 20 bp, Artificial sequence
  sgRNA
gcacagtatcaaaatacttg

SEQ ID NO: 101 - DNA, 20 bp, Artificial sequence
  sgRNA
gacaaagttgatgaaactgg

SEQ ID NO: 102 - DNA, 20 bp, Artificial sequence
  sgRNA
gccgatttccttatccaaag

SEQ ID NO: 103 - DNA, 20 bp, Artificial sequence
  sgRNA
gacccaagtgcatcaagaca

SEQ ID NO: 104 - DNA, 20 bp, Artificial sequence
  sgRNA
gcacttgggtctattctttc

SEQ ID NO: 105 - DNA, 20 bp, Artificial sequence
  sgRNA
gggcgactgttggatctgta

SEQ ID NO: 106 - DNA, 20 bp, Artificial sequence
  sgRNA
gtccagtaaaagctggagga

SEQ ID NO: 107 - DNA, 20 bp, Artificial sequence
  sgRNA
gagtggttctgaaatccaca

SEQ ID NO: 108 - DNA, 20 bp, Artificial sequence
  sgRNA
ggagagcaatgttaagctct

SEQ ID NO: 109 - DNA, 20 bp, Artificial sequence
  sgRNA
gactgtgtgcagagagcaac

SEQ ID NO: 110 - DNA, 20 bp, Artificial sequence
  sgRNA
gtcacttctcattacagttt

SEQ ID NO: 111 - DNA, 20 bp, Artificial sequence
  sgRNA
gatgccctgcagaggaaagg

SEQ ID NO: 112 - DNA, 20 bp, Artificial sequence
  sgRNA
ggcagagcgcttcagtgagc

SEQ ID NO: 113 - DNA, 20 bp, Artificial sequence
  sgRNA
gacagtgtgctgagagaccg

SEQ ID NO: 114 - DNA, 20 bp, Artificial sequence
  sgRNA
ggccggaaattcccagcttc

SEQ ID NO: 115 - DNA, 20 bp, Artificial sequence
  sgRNA
gtgtttcttttggtcttagg

SEQ ID NO: 116 - DNA, 20 bp, Artificial sequence
  sgRNA
gtgtttctccctttaagtct

SEQ ID NO: 117 - DNA, 20 bp, Artificial sequence
  sgRNA
ggcagccccaattctgctca

SEQ ID NO: 118 - DNA, 20 bp, Artificial sequence
  sgRNA
gatattagccgtgactcaga

SEQ ID NO: 119 - DNA, 20 bp, Artificial sequence
  sgRNA
gaagacaaagatgattttaa

SEQ ID NO: 120 - DNA, 20 bp, Artificial sequence
  sgRNA
ggtttcctacaaaagagtta

SEQ ID NO: 121 - DNA, 20 bp, Artificial sequence
  sgRNA
gctcattggttggtttctgc

SEQ ID NO: 122 - DNA, 20 bp, Artificial sequence
  sgRNA
gcctttctccaaaaggtatg

SEQ ID NO: 123 - DNA, 20 bp, Artificial sequence
  sgRNA
ggttagtatggctccgcgtg

SEQ ID NO: 124 - DNA, 20 bp, Artificial sequence
  sgRNA
gcgttacttccagattaacc

SEQ ID NO: 125 - DNA, 20 bp, Artificial sequence
  sgRNA
gcgttacttccagattaacc

SEQ ID NO: 126 - DNA, 20 bp, Artificial sequence
  sgRNA
gataaacctcttaggattac

SEQ ID NO: 127 - DNA, 20 bp, Artificial sequence
  sgRNA
ggaacgggctggtgttaaaa

SEQ ID NO: 128 - DNA, 20 bp, Artificial sequence
  sgRNA
gtcttcccttttcaacaatc

SEQ ID NO: 129 - DNA, 20 bp, Artificial sequence
  sgRNA
gaaaagggaagaccagcccc

SEQ ID NO: 130 - DNA, 20 bp, Artificial sequence
  sgRNA
gttagcatacaagacctttc

SEQ ID NO: 131 - DNA, 20 bp, Artificial sequence
  sgRNA
ggaggcggtgagtagtaagg

SEQ ID NO: 132 - DNA, 20 bp, Artificial sequence
  sgRNA
gaggaggcggtgagtagtaa

SEQ ID NO: 133 - DNA, 20 bp, Artificial sequence
  sgRNA
gaattttggacatgctaggg

SEQ ID NO: 134 - DNA, 20 bp, Artificial sequence
  sgRNA
gtgagcctgttttctcctct

SEQ ID NO: 135 - DNA, 20 bp, Artificial sequence
  sgRNA
ggttacctagaggagaaaac

SEQ ID NO: 136 - DNA, 20 bp, Artificial sequence
  sgRNA
gtatacaccttcataacctg

SEQ ID NO: 137 - DNA, 20 bp, Artificial sequence
  sgRNA
gcagtggccattgtgcagac

SEQ ID NO: 138 - DNA, 20 bp, Artificial sequence
  sgRNA
ggcacctggtgaaagaggca

SEQ ID NO: 139 - DNA, 20 bp, Artificial sequence
  sgRNA
gccaacccttgtgagcacgc

SEQ ID NO: 140 - DNA, 20 bp, Artificial sequence
  sgRNA
gagcacactcatccacgtcc

SEQ ID NO: 141 - DNA, 20 bp, Artificial sequence
  sgRNA
ggtgacaagggtgtcgtcta

SEQ ID NO: 142 - DNA, 20 bp, Artificial sequence
  sgRNA
gtgtcgtctatggaacggat

SEQ ID NO: 143 - DNA, 20 bp, Artificial sequence
  sgRNA
ggtaaactttgtctgaagtc

SEQ ID NO: 144 - DNA, 20 bp, Artificial sequence
  sgRNA
ggagttgggttatgtcttcc

SEQ ID NO: 145 - DNA, 20 bp, Artificial sequence
  sgRNA
gactcactgtcttgttctct

SEQ ID NO: 146 - DNA, 20 bp, Artificial sequence
  sgRNA
gagtgtttgtacatagatac

SEQ ID NO: 147 - DNA, 20 bp, Artificial sequence
  sgRNA
gcagaacggaataaaatgat

SEQ ID NO: 148 - DNA, 20 bp, Artificial sequence
  sgRNA
gatgactgctttggtaaatg

SEQ ID NO: 149 - DNA, 20 bp, Artificial sequence
  sgRNA
gattacccacttaccatggc

SEQ ID NO: 150 - DNA, 20 bp, Artificial sequence
  sgRNA
gaggaccagccatggtaagt

SEQ ID NO: 151 - DNA, 20 bp, Artificial sequence
  sgRNA
gctctgcagagtatattccc

SEQ ID NO: 152 - DNA, 20 bp, Artificial sequence
  sgRNA
gcatgtaggtgatgcagggc

SEQ ID NO: 153 - DNA, 20 bp, Artificial sequence
  sgRNA
gtttgtcatcttcatctcct

SEQ ID NO: 154 - DNA, 20 bp, Artificial sequence
  sgRNA
gtatgccgaatgtgttcccg

SEQ ID NO: 155 - DNA, 20 bp, Artificial sequence
  sgRNA
gaccttcctagaaggcaagg

SEQ ID NO: 156 - DNA, 20 bp, Artificial sequence
  sgRNA
gtccatttcaaagtaagcaa

SEQ ID NO: 157 - DNA, 20 bp, Artificial sequence
  sgRNA
gcaatggagcaccagtactc

SEQ ID NO: 158 - DNA, 20 bp, Artificial sequence
  sgRNA
gatgattggaaatgggaggc

SEQ ID NO: 159 - DNA, 20 bp, Artificial sequence
  sgRNA
gtcacaacagggcagcttga

SEQ ID NO: 160 - DNA, 20 bp, Artificial sequence
  sgRNA
gatggctatgtggatccttc

SEQ ID NO: 161 - DNA, 20 bp, Artificial sequence
  sgRNA
gggctcccgtgggcacttca

SEQ ID NO: 162 - DNA, 20 bp, Artificial sequence
  sgRNA
gaaaaccctgaagtgcccac

SEQ ID NO: 163 - DNA, 20 bp, Artificial sequence
  sgRNA
gaagattccccgggtgggcc

SEQ ID NO: 164 - DNA, 20 bp, Artificial sequence
  sgRNA
gggtgggcccggaacatctc

SEQ ID NO: 165 - DNA, 20 bp, Artificial sequence
  sgRNA
gattgcgatgcgctcatggc

SEQ ID NO: 166 - DNA, 20 bp, Artificial sequence
  sgRNA
gcgcgcggggggcatgttgg

SEQ ID NO: 167 - DNA, 20 bp, Artificial sequence
  sgRNA
gcctcctccaggcgcgcggg

SEQ ID NO: 168 - DNA, 20 bp, Artificial sequence
  sgRNA
gtcctagtgtagggaccggg

SEQ ID NO: 169 - DNA, 20 bp, Artificial sequence
  sgRNA
gagggttgggcgtgggggct

SEQ ID NO: 170 - DNA, 20 bp, Artificial sequence
  sgRNA
gtagaggtgcgtatctgtca

SEQ ID NO: 171 - DNA, 20 bp, Artificial sequence
  sgRNA
gttcgaggtgaaccattaat

SEQ ID NO: 172 - DNA, 20 bp, Artificial sequence
  sgRNA
gaggtcagaacaggagcgct

SEQ ID NO: 173 - DNA, 20 bp, Artificial sequence
  sgRNA
ggctctctgagtagtgcagg

SEQ ID NO: 174 - DNA, 20 bp, Artificial sequence
  sgRNA
gaatcatggaatcccttgca

SEQ ID NO: 175 - DNA, 20 bp, Artificial sequence
  sgRNA
gaacctttttattcctagga

SEQ ID NO: 176 - DNA, 20 bp, Artificial sequence
  sgRNA
gaggcagaacgtcgtaaaga

SEQ ID NO: 177 - DNA, 20 bp, Artificial sequence
  sgRNA
gttctcttccggcgaggaga

SEQ ID NO: 178 - DNA, 20 bp, Artificial sequence
  sgRNA
ggaggtggactcggagtgcg

SEQ ID NO: 179 - DNA, 20 bp, Artificial sequence
  sgRNA
gagatgggaaggacagaggc

SEQ ID NO: 180 - DNA, 20 bp, Artificial sequence
  sgRNA
gactttctcagagaaggtga

SEQ ID NO: 181 - DNA, 20 bp, Artificial sequence
  sgRNA
gaaccgacaaacagtcctgg

SEQ ID NO: 182 - DNA, 20 bp, Artificial sequence
  sgRNA
ggtcaggcaccactgccatc

SEQ ID NO: 183 - DNA, 20 bp, Artificial sequence
  sgRNA
gtcctctccccagggcccta

SEQ ID NO: 184 - DNA, 20 bp, Artificial sequence
  sgRNA
gtggacagataaagctcgaa

SEQ ID NO: 185 - DNA, 20 bp, Artificial sequence
  sgRNA
gctatgtgcctatcacaggg

SEQ ID NO: 186 - DNA, 20 bp, Artificial sequence
  sgRNA
gggatacctacctgaatcca

SEQ ID NO: 187 - DNA, 20 bp, Artificial sequence
  sgRNA
gggaggtgggggactccacg

SEQ ID NO: 188 - DNA, 20 bp, Artificial sequence
  sgRNA
gtcccctttgtagatctaag

SEQ ID NO: 189 - DNA, 20 bp, Artificial sequence
  sgRNA
ggagatcccatgacttctac

SEQ ID NO: 190 - DNA, 20 bp, Artificial sequence
  sgRNA
ggggaggggacacctacaga

SEQ ID NO: 191 - DNA, 20 bp, Artificial sequence
  sgRNA
gagattattctctgtatttt

SEQ ID NO: 192 - DNA, 20 bp, Artificial sequence
  sgRNA
gtcttaatgtctttccttta

SEQ ID NO: 193 - DNA, 20 bp, Artificial sequence
  sgRNA
gatcttcttctcggccctaa

SEQ ID NO: 194 - DNA, 20 bp, Artificial sequence
  sgRNA
gttcacaatgagttagaaga

SEQ ID NO: 195 - DNA, 20 bp, Artificial sequence
  sgRNA
ggacactgagatatatctat

SEQ ID NO: 196 - DNA, 20 bp, Artificial sequence
  sgRNA
gtccatggtagttgatctta

SEQ ID NO: 197 - DNA, 20 bp, Artificial sequence
  sgRNA
gctgcagccaagagctcttg

SEQ ID NO: 198 - DNA, 20 bp, Artificial sequence
  sgRNA
gattatccgaattcttagca

SEQ ID NO: 199 - DNA, 20 bp, Artificial sequence
  sgRNA
gacaatctgatgctatatct

SEQ ID NO: 200 - DNA, 20 bp, Artificial sequence
  sgRNA
ggtatattttccaagtcttg

SEQ ID NO: 201 - DNA, 20 bp, Artificial sequence
  sgRNA
gtggagagctgtctcaccag

SEQ ID NO: 202 - DNA, 20 bp, Artificial sequence
  sgRNA
gggtgtggaggtgtctgatg

SEQ ID NO: 203 - DNA, 20 bp, Artificial sequence
  sgRNA
ggtcatgcacaggtggcggc

SEQ ID NO: 204 - DNA, 20 bp, Artificial sequence
  sgRNA
gatggcacagctctgaagag

SEQ ID NO: 205 - DNA, 20 bp, Artificial sequence
  sgRNA
gctctggaagtgcaggcttg

SEQ ID NO: 206 - DNA, 20 bp, Artificial sequence
  sgRNA
ggtggtgaggtccgaaggag

SEQ ID NO: 207 - DNA, 20 bp, Artificial sequence
  sgRNA
ggaagggtggtgaggtccga

SEQ ID NO: 208 - DNA, 20 bp, Artificial sequence
  sgRNA
gcccacaggcattgcagacc

SEQ ID NO: 209 - DNA, 20 bp, Artificial sequence
  sgRNA
gaggaacgctaatggggacc

SEQ ID NO: 210 - DNA, 20 bp, Artificial sequence
  sgRNA
gtaccatctcgccgccacag

SEQ ID NO: 211 - DNA, 20 bp, Artificial sequence
  sgRNA
ggttcattgtcactaacatc

SEQ ID NO: 212 - DNA, 20 bp, Artificial sequence
  sgRNA
gaatgctgatcttcatcaaa

SEQ ID NO: 213 - DNA, 20 bp, Artificial sequence
  sgRNA
gaacttgtcctcccgccgcg

SEQ ID NO: 214 - DNA, 20 bp, Artificial sequence
  sgRNA
gttcttcataccaggaccag

SEQ ID NO: 215 - DNA, 20 bp, Artificial sequence
  sgRNA
gggaattgtgactccctgat

SEQ ID NO: 216 - DNA, 20 bp, Artificial sequence
  sgRNA
gctgcagaagaaaaagatac

SEQ ID NO: 217 - DNA, 20 bp, Artificial sequence
  sgRNA
gcgccacttttgggggtaag

SEQ ID NO: 218 - DNA, 20 bp, Artificial sequence
  sgRNA
gaacctagattttgagacag

SEQ ID NO: 219 - DNA, 20 bp, Artificial sequence
  sgRNA
gaattttcttcagcctctcc

SEQ ID NO: 220 - DNA, 20 bp, Artificial sequence
  sgRNA
gagggctgcgccacttttgg

SEQ ID NO: 221 - DNA, 20 bp, Artificial sequence
  sgRNA
ggctacccaaatatgaatca

SEQ ID NO: 222 - DNA, 20 bp, Artificial sequence
  sgRNA
ggacccccatatcctatggg

SEQ ID NO: 223 - DNA, 20 bp, Artificial sequence
  sgRNA
gctgcctaggatagcctcct

SEQ ID NO: 224 - DNA, 20 bp, Artificial sequence
  sgRNA
gacgcatgagccattctccc

SEQ ID NO: 225 - DNA, 20 bp, Artificial sequence
  sgRNA
gaagtgtactggggcatctg

SEQ ID NO: 226 - DNA, 20 bp, Artificial sequence
  sgRNA
ggagagagtttacttccgag

SEQ ID NO: 227 - DNA, 20 bp, Artificial sequence
  sgRNA
gtctttgtcctgaggcctta

SEQ ID NO: 228 - DNA, 20 bp, Artificial sequence
  sgRNA
gtggagtgctgcactggccc

SEQ ID NO: 229 - DNA, 20 bp, Artificial sequence
  sgRNA
gctgtgagtgaatgatgttg

SEQ ID NO: 230 - DNA, 20 bp, Artificial sequence
  sgRNA
gccagtgttttgagttctag

SEQ ID NO: 231 - DNA, 20 bp, Artificial sequence
  sgRNA
gtctacaagcataatcacac

SEQ ID NO: 232 - DNA, 20 bp, Artificial sequence
  sgRNA
gattatgcttgtagacaggt

SEQ ID NO: 233 - DNA, 20 bp, Artificial sequence
  sgRNA
gatggcgtagagggggaaaa

SEQ ID NO: 234 - DNA, 20 bp, Artificial sequence
  sgRNA
gataactgtgctggtccaga

SEQ ID NO: 235 - DNA, 20 bp, Artificial sequence
  sgRNA
gctatgacagtgtcacaatg

SEQ ID NO: 236 - DNA, 20 bp, Artificial sequence
  sgRNA
gtacaggcaggaggcaactg

SEQ ID NO: 237 - DNA, 20 bp, Artificial sequence
  sgRNA
gcaggaggcaactggggact

SEQ ID NO: 238 - DNA, 20 bp, Artificial sequence
  sgRNA
ggggtgcacagtcttgatgg

SEQ ID NO: 239 - DNA, 20 bp, Artificial sequence
  sgRNA
gtgtagccgttctgctccac

SEQ ID NO: 240 - DNA, 20 bp, Artificial sequence
  sgRNA
gtaccttggccactagtggg

SEQ ID NO: 241 - DNA, 20 bp, Artificial sequence
  sgRNA
gtggaacggcacatgtgtga

SEQ ID NO: 242 - DNA, 20 bp, Artificial sequence
  sgRNA
ggaacggcacatgtgtgatg

SEQ ID NO: 243 - DNA, 20 bp, Artificial sequence
  sgRNA
gacttcaggaattagtacgc

SEQ ID NO: 244 - DNA, 20 bp, Artificial sequence
  sgRNA
gaaggtcactgggcttagga

SEQ ID NO: 245 - DNA, 20 bp, Artificial sequence
  sgRNA
gtctgcagatgaaggtcact

SEQ ID NO: 246 - DNA, 20 bp, Artificial sequence
  sgRNA
gactccttgtctgaccccac

SEQ ID NO: 247 - DNA, 20 bp, Artificial sequence
  sgRNA
gaggaccattgtcatccgcc

SEQ ID NO: 248 - DNA, 20 bp, Artificial sequence
  sgRNA
ggatgtaatggagatagtcc

SEQ ID NO: 249 - DNA, 20 bp, Artificial sequence
  sgRNA
gtcacctgaaacagggggac

SEQ ID NO: 250 - DNA, 20 bp, Artificial sequence
  sgRNA
gtcggatcctgtctggtgag

SEQ ID NO: 251 - DNA, 20 bp, Artificial sequence
  sgRNA
ggagcccgaggaggggtttg

SEQ ID NO: 252 - DNA, 20 bp, Artificial sequence
  sgRNA
gggcgcaggccttcctggag

SEQ ID NO: 253 - DNA, 20 bp, Artificial sequence
  sgRNA
gaagaaacaccctctggctg

SEQ ID NO: 254 - DNA, 20 bp, Artificial sequence
  sgRNA
gtgtctgggcttggtgggat

SEQ ID NO: 255 - DNA, 20 bp, Artificial sequence
  sgRNA
gtgctgcctaatctgtcgga

SEQ ID NO: 256 - DNA, 20 bp, Artificial sequence
  sgRNA
gctccacagtgccagcgttc

SEQ ID NO: 257 - DNA, 20 bp, Artificial sequence
  sgRNA
gcgaagaagaatctaagagg

SEQ ID NO: 258 - DNA, 20 bp, Artificial sequence
  sgRNA
ggagaagcactgccgggata

SEQ ID NO: 259 - DNA, 20 bp, Artificial sequence
  sgRNA
ggttagcggagcagtgtcca

SEQ ID NO: 260 - DNA, 20 bp, Artificial sequence
  sgRNA
ggtgctggcgcaggagagcc

SEQ ID NO: 261 - DNA, 20 bp, Artificial sequence
  sgRNA
gaaaacagccaaggtttgta

SEQ ID NO: 262 - DNA, 20 bp, Artificial sequence
  sgRNA
gggtcaagtgcctgagaatg

SEQ ID NO: 263 - DNA, 20 bp, Artificial sequence
  sgRNA
gagttaccctacatacactc

SEQ ID NO: 264 - DNA, 20 bp, Artificial sequence
  sgRNA
gttcaggctgctgaccttca

SEQ ID NO: 265 - DNA, 20 bp, Artificial sequence
  sgRNA
ggaggttcctgtcaaaggag

SEQ ID NO: 266 - DNA, 20 bp, Artificial sequence
  sgRNA
ggtcttgggctcggccatac

SEQ ID NO: 267 - DNA, 20 bp, Artificial sequence
  sgRNA
gggtgaattcagtgtgagcc

SEQ ID NO: 268 - DNA, 20 bp, Artificial sequence
  sgRNA
gagcccaagaccgtctactg

SEQ ID NO: 269 - DNA, 20 bp, Artificial sequence
  sgRNA
gtatgtatcagtctcagtgg

SEQ ID NO: 270 - DNA, 20 bp, Artificial sequence
  sgRNA
ggtcgcttcagtcgtcagca

SEQ ID NO: 271 - DNA, 20 bp, Artificial sequence
  sgRNA
gccgcttgcagcaggtcttt

SEQ ID NO: 272 - DNA, 20 bp, Artificial sequence
  sgRNA
gcagcaggtctttgggttcc

SEQ ID NO: 273 - DNA, 20 bp, Artificial sequence
  sgRNA
gagtgtatacatactttata

SEQ ID NO: 274 - DNA, 20 bp, Artificial sequence
  sgRNA
gtatgcatctccatgaaaaa

SEQ ID NO: 275 - DNA, 20 bp, Artificial sequence
  sgRNA
gatctgtacacttttcttat

SEQ ID NO: 276 - DNA, 20 bp, Artificial sequence
  sgRNA
gcggggcgcactgggcagcg

SEQ ID NO: 277 - DNA, 20 bp, Artificial sequence
  sgRNA
gccaccgctgcccactgaga

SEQ ID NO: 278 - DNA, 20 bp, Artificial sequence
  sgRNA
gacggcaaaccctgccaggc

SEQ ID NO: 279 - DNA, 20 bp, Artificial sequence
  sgRNA
gccatgcagcagcacgccgt

SEQ ID NO: 280 - DNA, 20 bp, Artificial sequence
  sgRNA
gccgtggggggctactgcaa

SEQ ID NO: 281 - DNA, 20 bp, Artificial sequence
  sgRNA
acggaggctaagcgtcgcaa

SEQ ID NO: 282 - DNA, 20 bp, Artificial sequence
  sgRNA
cgcttccgcggcccgttcaa

SEQ ID NO: 283 - DNA, 20 bp, Artificial sequence
  sgRNA
atcgtttccgcttaacggcg

SEQ ID NO: 284 - DNA, 20 bp, Artificial sequence
  sgRNA
gtaggcgcgccgctctctac

SEQ ID NO: 285 - DNA, 20 bp, Artificial sequence
  sgRNA
ccatatcggggcgagacatg

SEQ ID NO: 286 - DNA, 20 bp, Artificial sequence
  sgRNA
tactaacgccgctcctacag

SEQ ID NO: 287 - DNA, 20 bp, Artificial sequence
  sgRNA
tgaggatcatgtcgagcgcc

SEQ ID NO: 288 - DNA, 20 bp, Artificial sequence
  sgRNA
gggcccgcataggatatcgc

SEQ ID NO: 289 - DNA, 7006 bp, Artificial sequence
  Vector
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgtaatagctcctgcatgggttttagagctagaaatagcaagttaaaataaggctagtccgttatcaacttgaaaaagtggcaccgagtcggtgctttttttctagaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggaagagcgagctcttctgttttagagctagaaatagcaagttaaaataaggctagtccgttatcaacttgaaaaagtggcaccgagtcggtgcttttttggtaccaggatcccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggatggagaggagacgcatcacctccgctgctcgccgggcgtacggccaccatgcccaagaagaagaggaaggtgtccaatctcctgactgttcaccagaacctccctgcgctgccagtagatgccactagcgatgaggtcaggaaaaatctcatggatatgtttagggatagacaggcgttttctgaacacacctggaaaatgctgcttagcgtgtgccgatcctgggcagcctggtgtaagctgaacaatcgcaaatggttccccgccgagccggaggacgtgcgcgattacctgctgtatctccaggcaagagggctggctgtcaagactatccagcagcacttgggccaactgaatatgctgcatcgacgcagcgggctcccccggcctagcgattcaaacgcagtctcccttgttatgaggagaattagaaaggaaaacgtagatgcgggtgagagggctaagcaggctctcgcttttgagcggactgatttcgaccaggtcagatccctgatggagaacagcgatcggtgccaggacatcaggaacctcgcatttctgggaattgcatataacacacttctgcgcatagctgagatcgcccggatcagagtgaaagacatcagtcgaacggacggcggccggatgcttattcatattggacgcacaaagacattggtcagcaccgctggcgttgaaaaggccttgtccctgggcgtaacgaagctggtggaaagatggatctcagtgtccggcgtggctgacgaccctaataattacttgttctgtcgagtgagaaaaaacggagtcgccgcgccctctgccaccagccaattgagtacacgggcccttgaagggatctttgaggcaacccaccgactcatatacggagccaaggatgacagtggccagaggtatctcgcctggtcaggtcattctgctagggtgggggccgcacgagacatggcgcgggcaggagtctccataccagagattatgcaagctggaggttggacaaatgtgaacatcgttatgaactatatccgcaatcttgactctgaaaccggggccatggtgagactgctcgaagatggtgactacccatacgatgttccagattacgcttaagaattcgatatcaagcttaataaaagatctttattttcattagatctgtgtgttggttttttgtgtggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 290 - DNA, 2209 bp, Homo sapiens
cccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggatggagaggagacgcatcacctccgctgctcgccggg

(19) United States
(12) Patent Application Publication
(10) Pub. No.: US 2020/0010903 A1
(43) Pub. Date: Jan. 9, 2020

(54) AAV-Mediated Direct In vivo CRISPR Screen in Glioblastoma

(71) Applicants: YALE UNIVERSITY, New Haven, CT (US); MASSACHUSETTS INSTITUTE OF TECHNOLOGY, Cambridge, MA (US)

(72) Inventors: Sidi Chen, Milford, CT (US); Ryan Chow, San Jose, CA (US); Christopher D. Guzman, Sandown, NH (US); Randall J. Platt, Basel (CH)

(21) Appl. No.: 16/489,595

(22) PCT Filed: Mar. 2, 2018
(86) PCT No.: PCT/US18/20729

§ 371 Date: Aug. 28, 2019

Related U.S. Application Data
(60) Provisional application No. 62/600,793, filed on Mar. 3, 2017.

(51) Int. Cl. C12Q 1/6886; C12N 9/22; C12N 15/11; C12N 15/90

(52) U.S. Cl. CPC C12Q 1/6886; C12N 9/22; C12N 15/11; C12N 15/907; C12N 2750/14131; C12N 2800/80; C12N 2320/12; C12N 2750/14143; C12Q 2600/106; C12Q 2600/156; C12N 2310/20

(57) ABSTRACT

The present invention includes novel compositions and methods for identifying driver mutations in glioblastoma. In one aspect, the invention includes an AAV-CRISPR library for identifying driver mutations in, and thus treatments for glioblastoma.

### CROSS-REFERENCE TO RELATED APPLICATION

The present application is entitled to priority under 35 U.S.C. § 119(e) to U.S. Provisional Patent Application No. 62/600,793 filed Mar. 3, 2017, which is hereby incorporated by reference in its entirety herein.

### STATEMENT REGARDING FEDERALLY SPONSORED RESEARCH OR DEVELOPMENT

This invention was made with government support under CA209992, CA121974, CA196530, GM04799, and GM007205 awarded by National Institutes of Health. The government has certain rights in the invention.

### BACKGROUND OF THE INVENTION

Glioblastoma (GBM) is the most frequent and most aggressive malignant primary brain tumor and is classified as grade IV by the World Health Organization. Primary or de novo GBMs are most common, which typically progress rapidly without recognizable symptoms. Secondary GBM can develop from lower-grade diffuse astrocytoma (grade II) and anaplastic astrocytoma (grade III). Unfortunately, with current standard of care, patients have a median survival of 14.6 months and five-year survival is merely 10%.

Extensive efforts have been undertaken to understand how genetic alterations promote transformation of normal cells in the brain into highly malignant glioma cells, particularly the roles of important drivers including oncogenes and tumor suppressor genes. Recent cancer genomic studies revealed hundreds of aberrations in GBM patients. Many of the newly discovered genes have never been associated with GBM; thus, their functional roles in gliomagenesis remain largely unknown. Furthermore, mutations can occur in novel combinations across different patients, which may lead to drastically different pathological features and therapeutic responses. Thus, a deeper functional understanding of gliomagenesis and a quantitative measurement of phenotypic effects across multiple genes in this process are both of central importance.

Achieving such a global, quantitative and functional understanding of gliomagenesis has been challenging, largely owing to the technological limitations of genetic manipulations in the past several decades. Genetically engineered mouse models with conditional alleles provide a powerful way to activate or inactivate specific genes to study their roles in tumor progression. Various transplant and genetic models of GBM have yielded insights on tumor progression in vivo. However, most current mouse model studies are limited to a single gene or a few genes, due to the technical challenges in generation of transgenic mice and breeding, even with blastocyst gene editing. Thus, the complexity of the cancer genome landscape far exceeds the availability of current mouse models as well as the ability to generate such models using conventional mouse genetics.

The recent development of RNA-guided genome editing harnessing the Cas9 endonuclease from the bacterial CRISPR (Clustered Regularly Interspaced Short Palidromic Repeats) has revolutionized genome editing. Cas9-mediated genome editing has empowered the rapid manipulation of oncogenes and tumor suppressor genes in vivo, such as Pten/p53 in liver cancer, Kras/p53/Lkb1, Elm4-Alk or Kras/p53/Nkx2.1 in lung adenocarcinoma, and Tet2/Dnmt3a/Runx1/Nf1/Ezh2 in acute myeloid leukemia (AML).

Despite these advances, to date, there lacks a comprehensive study using high-throughput genome editing to interrogate a collection of clinically relevant genes identified through cancer genome sequencing. In particular, the first genome atlas map of GBM uncovered 453 validated non-silent somatic mutations in 223 unique genes, which were further refined to a total of 71 significantly mutated genes (SMGs). Pan-cancer analyses involving thousands of patient tumor samples from multiple cancer types identified SMGs at the level of hundreds: 127 SMGs from 3,281 tumors in an analysis of 12 major cancer types, and 224 SMGs from 4,742 tumors in an analysis of 21 major cancer types. However, the relative strength of these SMGs as driving forces in tumorigenesis in vivo has not been systematically studied.

Direct in vivo high-throughput mutational analysis of functional cancer drivers in the mouse brain has been difficult, due to the nature of biological complexity in vivo. It is challenging to perform high-throughput analysis of mutants in autochthonous models of cancer; that is, models in which the tumors directly evolve from normal cells at the organ site in situ and without cellular transplantation.

There is a need in the art for compositions and methods for determining driver mutations in glioblastoma. A need also exists for high-throughput methods for analyzing mutants in autochthonous models of cancer. The present invention satisfies these needs.

### SUMMARY OF THE INVENTION

The present invention relates to compositions and methods for determining driver mutations in glioblastoma.

One aspect of the invention includes a method of determining treatment for a subject suffering from glioblastoma. The method comprises contacting a plurality of Adeno-Associated Virus-Clustered Regularly Interspaced Short Palidromic Repeats (AAV-CRISPR) vectors with a sample from the subject. The vectors comprise Cas9 and a plurality of nucleotide sequences homologous to a plurality of tumor suppressor genes (TSGs). A reaction mixture is generated. A plurality of nucleic acids isolated from the reaction mixture is sequenced and the data from the sequencing are analyzed as to identify any mutation in the plurality of nucleic acids. Treatment for the subject suffering from glioblastoma is determined based on presence and/or nature of any mutation in the plurality of nucleic acids.

Another aspect of the invention includes a method of determining at least one glioblastoma driver mutation in a sample. The method comprises contacting a plurality of AAV-CRISPR vectors with the sample. The vectors comprise Cas9 and a plurality of nucleotide sequences homologous to a plurality of tumor suppressor genes (TSGs). A reaction mixture is generated. A plurality of nucleic acids isolated from the reaction mixture are sequenced and the sequencing data are analyzed as to identify any glioblastoma driver mutation therein.

Yet another aspect of the invention includes an AAV-CRISPR library comprising a plurality of AAV vectors comprising Cas9 and a plurality of nucleic acids homologous to a plurality of Tumor Suppressor Gene (TSGs). Still another aspect of the invention includes an AAV-CRISPR library comprising a plurality of AAV vectors comprising Cas9 and a plurality of nucleic acids homologous to a plurality of Tumor Suppressor Gene (TSGs), wherein the plurality of nucleic acids comprises at least one selected from the group consisting of SEQ ID NOs. 1-280. Another aspect of the invention includes an AAV-CRISPR library comprising a plurality of AAV vectors comprising Cas9 and a plurality of nucleic acids homologous to a plurality of Tumor Suppressor Gene (TSGs), wherein the plurality of nucleic acids comprises SEQ ID NOs. 1-280.

Another aspect of the invention includes a kit for determining at least one driver mutation in a glioblastoma sample comprising an AAV-CRISPR library comprising a plurality of AAV vectors comprising Cas9 and a plurality of nucleic acids homologous to a plurality of Tumor Suppressor Gene (TSGs), reagents for measuring the at least one driver mutation, and instructional material for use thereof.

Yet another aspect of the invention includes a kit for determining at least one driver mutation in a glioblastoma sample comprising an AAV-CRISPR library comprising a plurality of AAV vectors comprising Cas9 and a plurality of nucleic acids homologous to a plurality of Tumor Suppressor Gene (TSGs), reagents for measuring the at least one driver mutation, and instructional material for use thereof, wherein the plurality of nucleic acids comprises at least one selected from the group consisting of SEQ ID NOs. 1-280.

Still another aspect of the invention includes a kit for determining at least one driver mutation in a glioblastoma sample comprising an AAV-CRISPR library comprising a plurality of AAV vectors comprising Cas9 and a plurality of nucleic acids homologous to a plurality of Tumor Suppressor Gene (TSGs), reagents for measuring the at least one driver mutation, and instructional material for use thereof, wherein the plurality of nucleic acids comprises SEQ ID NOs. 1-280.

Another aspect of the invention includes a method of determining at least one glioblastoma driver mutation in vivo in a glioblastoma-affected subject. The method comprises administering into the brain of the subject a plurality of AAV-CRISPR vectors. The AAV-CRISPR vectors comprise Cas9 and a plurality of short guide RNAs (sgRNAs) homologous to a plurality of tumor suppressor genes (TSGs). A plurality of nucleic acids isolated from the subject's glioblastoma are sequenced and analysis of the sequencing data indicates whether any glioblastoma driver mutation is present in the subject's glioblastoma.

Yet another aspect of the invention includes a vector comprising an adeno-associated virus (AAV) genome, a U6 promoter gene, an sgRNA sequence, a Glial Fibrillary Acidic Protein (GFAP) promoter gene, and a Cre recombinase gene. Still another aspect of the invention includes a vector comprising an adeno-associated virus (AAV) genome, a U6 promoter gene, an sgRNA sequence, a Glial Fibrillary Acidic Protein (GFAP) promoter gene, and a Cre recombinase gene, wherein the GFAP promoter gene comprises the nucleic acid sequence of SEQ ID NO: 290. Another aspect of the invention includes a vector comprising the nucleic acid sequence of SEQ ID NO: 289.

Still another aspect of the invention includes a kit comprising a vector comprising the nucleic acid sequence of SEQ ID NO: 289, and instructional material for use thereof. Yet another aspect of the invention includes a kit comprising an adeno-associated virus (AAV) genome, a U6 promoter gene, an sgRNA sequence, a Glial Fibrillary Acidic Protein (GFAP) promoter gene, and a Cre recombinase gene, and instructional material for use thereof. Another aspect of the invention includes a kit comprising an adeno-associated virus (AAV) genome, a U6 promoter gene, an sgRNA sequence, a Glial Fibrillary Acidic Protein (GFAP) promoter gene, and a Cre recombinase gene, and instructional material for use thereof, wherein he GFAP promoter gene comprises the nucleic acid sequence of SEQ ID NO: 290.

In various embodiments of the above aspects or any other aspect of the invention delineated herein, the plurality of nucleotide sequences homologous to a plurality of TSGs comprises at least one selected from the group consisting of SEQ ID NOs. 1-280. In one embodiment, the plurality of nucleotide sequences homologous to a plurality of TSGs comprises SEQ ID NOs. 1-280. In another embodiment, the plurality of sgRNAs comprises at least one selected from the group consisting of SEQ ID NOs. 1-280. In yet another embodiment, the plurality of sgRNAs comprises SEQ ID NOs. 1-280.

In one embodiment, the sequencing comprises targeted capture sequencing.

In one embodiment, the mutation comprises a nucleotide insertion. In another embodiment, the insertion comprises more than one nucleotide base. In yet another embodiment, the mutation comprises a nucleotide deletion. In still another embodiment, the deletion comprises more than one nucleotide base.

In one embodiment, the sample comprises a plurality of glioma cells from the subject. In another embodiment, the sample comprises a tumor from the subject.

In certain embodiments, any one of the methods of the invention further comprise monitoring cell proliferation in the reaction mixture.

In one embodiment, the subject is a mammal. In another embodiment, the mammal is a mouse or a human.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1D are a series of images and plots illustrating an AAV-CRISPR based pooled mutagenesis for high-throughput autochthonous analysis of glioblastoma tumor suppression. FIG. 1A is a schematic of the overall experimental design. The top panel shows AAV-mTSG library design, synthesis and production and the bottom panel shows stereotaxic injection of viral library and subsequent analysis. FIG. 1B shows MRI imaging of brains of mice stereotaxically injected with PBS, AAV-vector or AAV-mTSG library. Arrowheads indicate brain tumors. FIG. 1C shows quantification of tumor size of MRI imaging in volume (mm³). PBS, n=2; Vector, n=6; mTSG, n=18). Two-tailed T test, p=0.018, mTSG vs. vector or PBS; Two-tailed T test, p=0.5, vector vs. PBS. FIG. 1D shows Kaplan-Meier curves for overall survival of mice stereotaxically injected with PBS, AAV-vector or AAV-mTSG library. Log-rank (LR) test, p<2.2e-16, mTSG vs. vector or PBS; LR test, p=1, vector vs PBS.

FIGS. 2A-2D are a series of plots and images showing histopathological analysis of AAV-mTSG induced mouse GBM. FIG. 2A shows representative marker staining of mouse brain sections in PBS, vector and mTSG groups. Top panel, Cas9 staining, arrowheads indicate Cas9-positive cells in the injected brain regions (vector mice) and tumors (mTSG mice); Middle panel, GFAP staining, arrows indicate representative astrocytes; Bottom panel, Ki67 staining, arrows indicate representative proliferative cells. FIG. 2B shows representative full slidescan images of endpoint histology (H&E) of mouse brain sections of PBS, vector and mTSG groups. Arrow indicates brain tumor. FIG. 2C shows quantification of tumor size of endpoint histology of mouse brain sections of PBS, vector and mTSG groups. PBS, n=3; Vector, n=7; mTSG, n=11). Two-tailed T test, p=0.0026, mTSG vs. vector or PBS. FIG. 2D shows representative higher magnification H&E images showing pathological features of AAV-mTSG induced mouse GBM. Arrowheads in the upper left panel indicate representative giant and aneuploidy cells with nuclei pleomorphic; Arrowheads in the lower left panel indicate representative necrotic regions; Arrows in the upper right panel indicate representative endothelial cells and angiogenesis; Arrows in the lower right panel indicate representative hemorrhage regions.

FIGS. 3A-3C are a series of plots and images showing representative mutation profiles of individual sgRNA target regions and GBM samples. FIG. 3A shows alleles observed at the genomic region targeted by Mll2 sgRNA 4 in representative PBS, vector, and mTSG samples. The percentage of total reads that correspond to each allele is indicated on the right. FIG. 3B is a set of bar plots of two representative mTSG brain samples with variant frequency in significantly mutated sgRNA target regions. Sum variant frequency is the cumulative frequency of all detected variants for a particular sgRNA. FIG. 3C is a series of boxplots of the number of samples cut by each of the 5 same-gene targeting sgRNAs, grouped by target gene.

FIGS. 4A-4C are a series of plots illustrating integrative analysis of functional mutations in driving tumorigenesis. FIG. 4A illustrates the gene-level mutational landscape of AAV-mTSG induced primary mouse GBM. Center: Tile chart depicting the mutational landscape of primary brain samples from LSL-Cas9 mice infected with the AAV-mTSG library (n=25), AAV-vector (n=3) or PBS (n=4). Genes are grouped and colored according to their functional classifications, as noted in the legend in the top-right corner. Top: Bar plots of the total number of significantly mutated genes identified in each AAV-mTSG sample. Right: Bar plots of the percentage of GBM samples that were called as significantly mutated for each gene. Left: Heatmap of the number of unique significantly mutated sgRNAs (SMSs) for each gene. Bottom: Stacked bar plots describing the type of indels observed in each sample, color-coded according to the legend in the bottom-right corner. FIGS. 4B-4C illustrate comparative cancer genomics in GBM using the TCGA (FIG. 4B) and Yale Glioma (FIG. 4C) datasets. Scatterplot of population-wide mutant frequencies for the genes in the mTSG library, comparing AAV-mTSG treated mouse brain samples to human samples. Representative strong drivers in both species are labeled, with gene names color-coded based on their functional classification (as in FIG. 4A). FIG. 4B shows mutant frequencies in AAV-mTSG treated mouse brain samples correlated with patients in the TCGA GBM dataset (Pearson correlation R=0.402, p=0.002). FIG. 4C shows mutant frequencies in AAV-mTSG treated mouse brain samples correlated with patients in the Yale Glioma dataset (Yale Glio) (Pearson correlation R=0.318, p=0.028).

FIGS. 5A-5F are a series of plots and images showing co-occurrence analysis of mutations identified in GBM samples. FIG. 5A, upper-left half, shows the number of samples with a mutation in both specified genes. FIG. 5A, lower-right half, shows the −log₁₀ p-values by hypergeometric test to evaluate whether specific pairs of genes are statistically significantly co-mutated. FIG. 5B is a scatterplot of the number of samples with a particular pair of mutations, plotted against −log₁₀ p-values. FIG. 5C is a set of Venn diagrams showing the strong co-occurrence of mutations in Kdm5c and Gata3 (left), as well as B2m and Pik3r1 (right). FIG. 5D, upper-left half, is a heatmap of the pairwise Pearson correlation of sum % variant frequency for each gene, averaged across sgRNAs. FIG. 5D, lower-right half, is a heatmap of −log₁₀ p-values by t-distribution to evaluate the statistical significance of the pairwise correlations. FIG. 5E is a plot of pairwise Pearson correlations plotted against −log₁₀ p-values. FIG. 5F is a scatterplot comparing sum % variant frequency, averaged across sgRNAs, for Rb1 and Tgfbr2. The Pearson correlation coefficient is noted on the plot.

FIGS. 6A-6D are a series of images showing additional data of massively parallel GBM suppressor analysis by AAV-CRISPR library mediated pooled mutagenesis. FIG. 6A shows an AAV vector that contains a cassette expressing Cre recombinase under GFAP promoter, a p53 sgRNA under U6 promoter, and an empty cassette for expression of custom cloned sgRNA(s). FIG. 6B shows a plasmid library representation of the AAV-CRISPR mTSG library. FIG. 6C shows a representative AAV-mTSG injected mouse showing macrocephaly. FIG. 6D shows dissected PBS, AAV-vector and AAV-mTSG mouse whole brains (left panel) and sections (right panel) visualized under a fluorescent stereoscope.

FIG. 7 is a series of full-spectrum MRI images of representative mouse brains in PBS, vector and mTSG group.

FIGS. 8A-8B are a series of full-scan histology images of special staining of mouse brains sections in vector and mTSG groups. FIG. 8A shows (panels from top to bottom) Luxol fast blue Cresyl violet (LFB/CV) staining and Wight Giemsa staining. FIG. 8B shows Masson staining and Alcian blue Periodic acid—Schiff (AB/PAS) staining.

FIG. 9 is a global heatmap of sum indel frequency across all targeted capture samples. Each row represents the sum indel frequencies of one sgRNA across samples. Each column is a sample from brain (targeted organ) and liver (non-targeted organ) of mice stereotaxically injected with all mTSG, vector or PBS.

FIG. 10 is a heatmap of indel size distribution of all sgRNAs and mSMGs in GBM mice induced with AAV-CRISPR mTSG library. Metaplot of indel sizes of all sgRNAs of each mouse in a row; negative number of base pairs indicate deletions; positive numbers of basepairs indicate insertions; colors as depicted in key (left subpanel) indicates the relative abundance of indels of a particular size in a particular mouse.

FIGS. 11A-11H are a series of plots showing mutational oncotypes of all GBM mice induced with AAV-CRISPR mTSG library. Waterfall plots of significantly mutated sgRNA sites across all mTSG brain samples, sored by sum variant frequency. The extensive mutational landscape in theses samples shows strong positive selection for LOF in gliomagenesis in the brains of these mice.

FIGS. 12A-12B are a series of images illustrating the testing of driver combinations with sgRNA minipool. FIG. 12A is a schematic representation of the experimental design. Mixtures of five sgRNAs targeting each gene were cloned as sgRNA minipool into the same astrocyte-specific AAV-CRISPR vector. After packaging, AAV minipools were stereotaxically injected into the ventricle of LSL-Cas9 mice. Survival and histology analysis followed injection. FIG. 12B shows H&E staining images of brain slices from injected mice at 3 month post injection. All of the uninjected (n=5), sg-YFP (n=4) and sg-Trp53 (n=6) mice were devoid of any observable tumor and survive with good body conditions. In contrast, 50% ( 2/4) of the mice receiving sg-Trp53; sg-Nf1 minipool, 75% (¾) of the mice receiving sg-Trp53; sg-Nf1; sg-Pten minipool, and 75% (¾) of the mice receiving sg-Trp53; sg-Pten; sg-Rb1 minipool developed macrocephaly, poor body condition score and large tumors. Chi-square test, one tailed, p=0.011 for sg-Trp53; sg-Nf1, p=0.001 for both sg-Trp53; sg-Nf1; sg-Pten GBM AAV CRISPR screen 34 and sg-Trp53; sg-Pten; sg-Rb1, and not significant for sg-Trp53, all vs uninjected+sg-YFP. Scale bar is 0.1 mm.

FIGS. 13A-13E are a series of tables showing the sgRNA spacer sequences in the mTSG library.

FIG. 14 is a table showing MRI tumor size statistics of GBM mice induced with AAV-CRISPR mTSG library. T=tumor observed, noT=tumor not observed, ud=unable to determine with resolution of MRI, depth measured by number of frames, frame-distance=0.5 mm.

FIG. 15 is a series of tables showing survival statistics of GBM mice induced with AAV-CRISPR mTSG library.

FIG. 16 is a series of tables showing histology tumor size statistics of GBM mice induced with AAV-CRISPR mTSG library.

FIGS. 17A-17F are a series of tables showing mTSG-Amplicon capture probe design of targeted region coordinates and coverage.

FIG. 18 is a set of tables showing sample metadata for targeted capture sequencing of GBM mice induced with AAV-CRISPR mTSG library.

FIGS. 19A-19C are a series of plots and images illustrating that AAV-mTSG induced brain tumors recapitulate pathological features of GBM. FIG. 19A top panel, shows representative H&E brain sections from PBS, AAV-vector and AAV-mTSG injected mice. Arrowheads indicate brain tumors. Scale bar=1 mm. FIG. 19A lower panels, show representative images of brain sections from PBS, AAV-vector and AAV-mTSG injected mice stained by Cas9, GFP, GFAP, and Ki67 immunohistochemistry. Cas9 IHC, arrowheads indicate Cas9-positive cells; GFP IHC, arrowheads indicate GFP-positive cells; GFAP IHC, representative GFAP-positive astrocytes in PBS, AAV-vector and AAV-mTSG injected mice (arrows), as well as representative cancer cells in AAV-mTSG injected mice (arrowheads); Ki67 IHC, arrowheads indicate representative proliferative cells, which are mostly in tumors (AAV-mTSG) or scattered in tumor-adjacent brain regions (AAV-mTSG). Scale bar=0.25 mm. FIG. 19B shows quantification of tumor sizes±s.e.m. found in H&E brain sections from PBS, AAV-vector and AAV-mTSG injected mice. Two-tailed Welch's t-test, t₁₀=3.97, p=0.003, mTSG vs. vector or PBS (PBS, n=3; Vector, n=7; mTSG, n=11). FIG. 19C shows representative higher magnification H&E images showing pathological features of AAV-mTSG induced brain tumors. Clockwise from top left: arrowheads, giant aneuploid cells with pleomorphic nuclei; arrows, endothelial cells and angiogenesis; arrows, hemorrhagic regions; black arrowheads, necrotic regions. Similar features were observed in human GBM patient sections from Yale Glioma tissue bank (FIGS. 20A-20B). Scale bar=0.5 mm.

FIGS. 20A-20B are representative histopathology images of human GBM. FIG. 20A shows representative images of H&E stained brain sections from human GBM patient samples from Yale Glioma tissue bank. Images from the three rows represent GBM with significant mutations in NF1, PTEN and RB1, respectively. Pathological features such as giant aneuploid cells with pleomorphic nuclei, angiogenesis, necrosis and hemorrhage were evident in these tumors. Scale bar=0.5 mm. FIG. 20B shows representative images of anti-GFAP stained brain sections from human GBM patient samples from Yale Glioma tissue bank. Images from the two rows represent GBM with significant mutations in PTEN and RB1, respectively. PTEN tumors were mostly GFAPpositive. RB1 tumors have mixtures of GFAP-positive and GFAP-negative cells. NF1 tumors were not shown due to availability of GFAP staining sections. Scale bar=0.5 mm.

FIGS. 21A-21E are a series of plots and images illustrating targeted captured sequencing of sgRNA sites in AAV-mTSG induced mouse GBM. FIG. 21A shows indel variants observed at the genomic region targeted by Mll2 sgRNA 4 in representative PBS, AAV-vector, and AAV-mTSG injected mouse brain samples. FIG. 21B is a set of bar plots of variant frequencies in significantly mutated sgRNA target regions from two representative AAV-mTSG injected mouse brain samples. FIG. 21C is a heatmap of variant frequency across all targeted capture samples (n=41). Rows denote individual sgRNAs, while columns correspond to samples from mice stereotaxically injected with PBS, AAV-vector, or AAV-mTSG. The liver was considered an off-target organ and thus was used as a background control. Bar plots of the mean variant frequencies for each sgRNA (right panel) and each sample (bottom panel) are shown. FIG. 21D is a dot plot of mean variant frequency±s.e.m., grouped by treatment condition and tissue type. AAV-mTSG injected brains had significantly higher mean variant frequencies (2.087±0.429, n=25) compared to vector (0.005±0.001, n=3) or PBS (0.003±0.001, n=4) injected brains (two-tailed Welch's t-test, t₂₄=4.85 and t₂₄=4.86, p=6.03*10⁻⁵ and p=5.96*10⁻⁵ for mTSG vs. vector and mTSG vs. PBS). Comparing brain vs. liver in AAV-mTSG injected mice, mean variant frequencies of brains (2.087±0.429) were significantly higher than livers (0.309±0.261, n=4) (t_{21.48}=3.54, p=0.002). FIG. 21E shows indel size distribution for all filtered variants in each mTSG brain sample (n=25).

FIGS. 22A-22C are a series of images illustrating early time point analysis of sgRNA cutting efficiency by molecular inversion probe sequencing. FIG. 22A is a heatmap of sum variant frequencies for each sgRNA across the 3 in vivo infection replicates. Each row denotes one gene, while each column corresponds to a specific sgRNA and replicate. Variant frequencies are square-rooted to improve visibility. FIG. 22B shows dissected whole brain from an AAV-mTSG injected mouse for early time point analysis, visualized under a fluorescent stereoscope. GFP is shown as an overlay on the brightfield image. FIG. 22C is a Venn diagram detailing the overlap between cutting sgRNAs identified in early-stage mutagenesis and late-stage GBMs. Differences in the identified cutting sgRNAs were likely due to differential selection pressures, insufficient time for CRISPR mutagenesis to occur in early time point brains, and/or allele frequencies below detection limit of capture sequencing.

FIGS. 23A-23E are a series of plots and images illustrating additional analysis of mutational signatures. FIG. 23A shows scatterplots of the number of samples with an SMS call per sgRNA (left) or SMG call per gene (right), using two different thresholds for calling SMSs. In conjunction with the FDR approach, the use of either a flat 5% or 10% variant frequency cutoff did not affect the results at either the sgRNA or gene level. Spearman correlation coefficients and associated p-values are shown on the plots. FIG. 23B-23E show Gaussian kernel density estimate of variant frequencies within each mTSG brain sample. The number of peaks in the kernel density estimate is an approximation for the clonality of each sample. From this analysis, most ( 20/22) samples appeared to be composed of multiple clones, with only two (mTSG brain 15, mTSG brain 20) monoclonal samples. Of note, 3/25 sequenced mTSG brain samples did not have sufficient high-frequency variants for clustering analysis.

FIGS. 24A-24G are a series of plots and images showing co-mutation analysis uncovers synergistic gene pairs in GBM. FIG. 24A upper-left half, is a heatmap of pairwise mutational co-occurrence rates. FIG. 24A, lower-right half, is a heatmap of −log₁₀ p-values by hypergeometric test for statistical co-occurrence. FIG. 24B is a scatterplot of the co-occurrence rate of each gene pair, plotted against −log₁₀ p-values. FIG. 24C is a series of Venn diagrams showing representative strongly co-occurring mutated gene pairs such as Kdm5c and Gata3 (co-occurrence rate=77.8%, hypergeometric test, p=6.04*10⁻⁶), B2m and Pik3r1 (70.0%, p=2.28*10⁻⁵), as well as Nf1 and Pten (85.7%, p=7.53*10⁻⁸). FIG. 24D upper-left half, is a heatmap of the pairwise Spearman correlation of variant frequency for each gene, summed across sgRNAs. FIG. 24D lower-right half, is a heatmap of −log₁₀ p-values to evaluate the statistical significance of the pairwise correlations. FIG. 24E is a scatterplot of pairwise Spearman correlations plotted against −log₁₀ p-values. FIGS. 24F-24G are scatterplots showing representative strongly correlated gene pairs when comparing variant frequencies summed across sgRNAs, such as Nf1+Pten (FIG. 24F) and Cdkn2a+Ctcf (FIG. 24G). Spearman correlation coefficients are noted on the plot.

FIGS. 25A-25F are a series of plots illustrating additional analysis of co-mutated pairs and exome sequencing. FIG. 25A is a scatterplot of the co-occurrence rate of a given mutation pair, plotted against −log10 p-values. All pairs involving Trp53 were excluded from this analysis. FIG. 25B is a scatterplot of pairwise Spearman correlations plotted against −log10 p-values. All pairs involving Trp53 were excluded from this analysis. FIG. 25C is a scatterplot of the co-occurrence rate of a given mutation pair in the TCGA human GBM dataset, plotted against −log10 p-values. FIG. 25D is a Venn diagram of co-occurring pairs identified in mouse GBM (Benjamini-Hochberg adjusted p<0.05, either co-occurrence or Spearman correlation analysis) and/or in human GBM (p<0.05). 7 gene pairs were found to be significant in both mouse and human GBM. The overlap between the two datasets was significant (hypergeometric test, p=0.001). FIGS. 25E-25F show whole-exome analysis of possible off-target mutations generated by AAV-CRISPR mTSG (n=7). Chromosomal map of potential off-targets in AAV-CRISPR mTSG brain samples. Indels in mTSG genes are marked in black, while possible off-target mutations and AAV insertions are marked in dark grey.

FIGS. 26A-26E are a series of images illustrating validation of driver combinations. FIG. 26A is a schematic representation of the experimental design. Mixtures of five sgRNAs targeting each gene were cloned as minipools into the astrocyte-specific AAV-CRISPR vector. After packaging, AAV minipools were stereotaxically injected into the lateral ventricle of LSL-Cas9 mice. FIGS. 26B-26E show end-point histology (H&E) of representative brain sections from mice treated with AAV sgRNA minipools or relevant controls. In this end-point analysis, mice were euthanized when either macrocephaly or poor body condition score (<2) was observed, with survival time ranging from 3 to 11 months. Treatments are indicated to the left of each image. Arrowheads indicate the presence of brain tumors. The proportion of tumor-bearing to total mice is indicated in the top right corner of the images. Scale bar=0.5 mm.

FIGS. 27A-27B are a series of images illustrating GFAP immunohistochemical characterization of brain sections from mice treated with AAV various sgRNA minipools. Brain tumors in Nf1, Nf1; Pten, and Nf1; B2m mice were strongly positive for GFAP, while tumors in Nf1; Mll3 mice were positive at an intermediate level. Brain tumors in Rb1, Rb1; Pten, and Rb1; Zc3h13 mice contained a mixture of GFAP positive and negative cells, similar to the GFAP staining pattern with human patient GBM samples. Brain tumors in Mll2 mice were variably GFAP positive. Scale bar=0.5 mm.

FIGS. 28A-28D illustrate additional data related to the study. FIG. 28A shows Kaplan-Meier overall survival curves for mice injected with control (n=9), B2m (n=4), Nf1 (n=8), and Nf1; B2m (n=4) AAV minipools. All control and B2m mice were tumor-free and survived the entire duration of the experiment; control and B2m curves are offset for visibility. Mice treated with Nf1; B2m AAVs had significantly worse survival compared to mice treated with Nf1 or B2m AAVs alone (Log-rank (LR) test, p=0.0067). FIG. 28B shows results from a T7E1 nuclease assay to confirm mutagenesis by CRISPR/Cas9 at the indicated target genes. Indel frequencies are indicated. FIGS. 28C-28D illustrate LentiCRISPR mTSG direct in vivo GBM screen. FIG. 28C shows IVIS imaging of mice injected with lenti-vector or lenti-mTSG library, showing luminescence in the brains of a fraction of lenti-mTSG injected mice, but not in vector injected mice. Mice were imaged at 6.5 months post injection (mpi), where 4/18 mice imaged were luciferase positive (10 were shown). These 4 mice were sacrificed as they developed poor body conditions and brain tumors, before the end of 10 mpi. Mice were imaged again at 11 mpi, where 6/14 mice imaged luciferase positive, which were subsequently sacrificed as they developed poor body conditions and brain tumors. FIG. 28D shows Kaplan-Meier curves for overall survival (OS) of mice injected with PBS (n=2), lenti-vector (n=5) or lenti-mTSG library (n=18). OS for PBS and vector groups were both 100%, where the curves are dashed and slightly offset for visibility. LR test, p<0.0239, mTSG vs. vector or PBS; LR test, p=1, vector vs. PBS.

FIGS. 29A-29H are a series of plots and images illustrating transcriptional profiling of mouse GBM driver combinations. FIG. 29A is a schematic of mouse GBM RNA-seq experimental design. Rb1 or Nf1 AAV minipools were stereotaxically injected into the lateral ventricle of LSL-Cas9 mice. Cell lines were derived from mouse GBMs by single-cell isolation. Additional driver mutations were introduced by lentiCRISPR where applicable. Cells were then transcriptionally profiled by RNA-seq (n=3 samples per condition). FIG. 29B is a Volcano plot comparing gene expression profiles in Rb1 to Nf1 mutant GBM cells. 616 genes were significantly higher in Rb1 cells, and 982 genes were significantly higher in Nf1 cells. FIG. 29C shows enriched gene ontology categories among Nf1-high genes. FIG. 29D shows enriched gene ontology categories among Rb1-high genes. FIG. 29E is a Volcano plot comparing Nf1; Mll3 mutant to Nf1 mutant GBM cells. 522 genes were significantly higher in Nf1; Mll3 cells, and 175 genes were significantly higher in Nf1 cells. FIG. 29F shows enriched gene ontology categories among Nf1; Mll3-high genes. FIG. 29G is a Volcano plot comparing Rb1; Zc3h13 mutant to Rb1 mutant GBM cells. 703 genes were significantly higher in Rb1; Zc3h13, and 166 genes were significantly higher in Rb1 cells. FIG. 29H shows enriched gene ontology categories among Rb1; Zc3h13-high genes. Differentially expressed genes were defined as Benjamini-Hochberg adjusted p<0.05 and log fold change≥1 or ≤−1.

FIGS. 30A-30I are a series of plots and images illustrating transcriptional profiling of mouse GBM driver combinations in the presence and absence of a chemotherapeutic agent. FIG. 30A is a schematic of drug treatment RNA-Seq experimental design. Rb1, Rb1; Pten, and Rb1; Zc3h13 GBM cells were treated with either temozolomide (TMZ) or DMSO. Cells were then subjected to phenotypic analysis and RNA-seq. FIGS. 30B-30C show survival fraction±s.e.m of Rb1, Rb1; Pten, and Rb1; Zc3h13 cells with 1 mM (FIG. 30B) or 2 mM (FIG. 30C) TMZ treatment. Individual cell replicates are shown (n=3 for all conditions). FIG. 30B shows Rb1; Pten and Rb1; Zc3h13 cells had significantly higher survival fractions than Rb1 cells with 1 mM TMZ (two-sided t-test, p=6.20*10⁻⁶ and p=1.94*10⁻⁵, respectively). FIG. 30C shows Rb1; Pten and Rb1; Zc3h13 cells had significantly higher survival fractions than Rb1 cells with 2 mM TMZ (two-sided t-test, p=9.06*10⁻⁷ and p=2.84*10⁻⁶, respectively). Individual cell replicates are shown (n=3 for all conditions). FIG. 30D is a Volcano plot comparing Rb1 cells treated with TMZ (dark blue) or DMSO. 352 genes were significantly higher in TMZ-treated cells (TMZ-induced genes), and 332 genes were significantly higher in DMSO-treated cells (TMZ-reduced genes). FIG. 30E is a Volcano plot comparing Rb1; Pten cells treated with TMZ or DMSO. 345 genes were significantly higher in TMZ-treated cells, and 313 genes were significantly higher in DMSO-treated cells. FIG. 30F is a Volcano plot comparing Rb1; Zc3h13 cells treated with TMZ or DMSO. 703 genes were significantly higher in TMZ-treated cells, and 166 genes were significantly higher in DMSO-treated cells. FIG. 30G is a heatmap of all differentially expressed genes among the TMZ vs. DMSO comparisons. Clustering was performed by average linkage using Pearson correlations. Values are shown in terms of z-scores, scaled by each gene. FIG. 30H is a Venn diagram of TMZ-reduced genes for each tested genotype. While 69 genes were similarly downregulated among all 3 genotypes upon TMZ treatment, the differential expression signatures were nevertheless distinct, suggesting differential responses to TMZ treatment. FIG. 30I is a Venn diagram of TMZ-induced genes for each tested genotype. Though 42 genes were consistently upregulated in all 3 groups upon TMZ treatment, numerous transcriptional differences were nevertheless apparent, suggesting differential responses to TMZ treatment. Differentially expressed genes were defined as Benjamini-Hochberg adjusted p<0.05 and log fold change ≥1 or ≤−1.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein the term “amount” refers to the abundance or quantity of a constituent in a mixture.

As used herein, the term “bp” refers to base pair.

The term “complementary” refers to the degree of anti-parallel alignment between two nucleic acid strands. Complete complementarity requires that each nucleotide be across from its opposite. No complementarity requires that each nucleotide is not across from its opposite. The degree of complementarity determines the stability of the sequences to be together or anneal/hybridize. Furthermore various DNA repair functions as well as regulatory functions are based on base pair complementarity.

The term “CRISPR/Cas” or “clustered regularly interspaced short palindromic repeats” or “CRISPR” refers to DNA loci containing short repetitions of base sequences followed by short segments of spacer DNA from previous exposures to a virus or plasmid. Bacteria and archaea have evolved adaptive immune defenses termed CRISPR/CRISPR-associated (Cas) systems that use short RNA to direct degradation of foreign nucleic acids. In bacteria, the CRISPR system provides acquired immunity against invading foreign DNA via RNA-guided DNA cleavage.

The “CRISPR/Cas9” system or “CRISPR/Cas9-mediated gene editing” refers to a type II CRISPR/Cas system that has been modified for genome editing/engineering. It is typically comprised of a “guide” RNA (gRNA) and a non-specific CRISPR-associated endonuclease (Cas9). “Guide RNA (gRNA)” is used interchangeably herein with “short guide RNA (sgRNA)” or “single guide RNA (sgRNA). The sgRNA is a short synthetic RNA composed of a “scaffold” sequence necessary for Cas9-binding and a user-defined ˜20 nucleotide “spacer” or “targeting” sequence which defines the genomic target to be modified. The genomic target of Cas9 can be changed by changing the targeting sequence present in the sgRNA.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

A “mutation” as used herein is a change in a DNA sequence resulting in an alteration from a given reference sequence (which may be, for example, an earlier collected DNA sample from the same subject). The mutation can comprise deletion and/or insertion and/or duplication and/or substitution of at least one deoxyribonucleic acid base such as a purine (adenine and/or thymine) and/or a pyrimidine (guanine and/or cytosine). Mutations may or may not produce discernible changes in the observable characteristics (phenotype) of an organism (subject).

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “oligonucleotide” typically refers to short polynucleotides, generally no greater than about 60 nucleotides. It will be understood that when a nucleotide sequence is represented by a DNA sequence (i.e., A, T, G, C), this also includes an RNA sequence (i.e., A, U, G, C) in which “U” replaces “T”.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polynucleotide” includes DNA, cDNA, RNA, DNA/RNA hybrid, anti-sense RNA, siRNA, miRNA, snoRNA, genomic DNA, synthetic forms, and mixed polymers, both sense and antisense strands, and may be chemically or biochemically modified to contain non-natural or derivatized, synthetic, or semisynthetic nucleotide bases. Also, included within the scope of the invention are alterations of a wild type or synthetic gene, including but not limited to deletion, insertion, substitution of one or more nucleotides, or fusion to other polynucleotide sequences.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The term “promoter” as used herein is defined as a DNA sequence recognized by the synthetic machinery of the cell, or introduced synthetic machinery, required to initiate the specific transcription of a polynucleotide sequence.

A “sample” or “biological sample” as used herein means a biological material from a subject, including but is not limited to organ, tissue, exosome, blood, plasma, saliva, urine and other body fluid. A sample can be any source of material obtained from a subject.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a genomic nucleic acid sequence that defines a portion of a nucleic acid to which a binding molecule may specifically bind under conditions sufficient for binding to occur.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny. In certain embodiments, “transfected” means an exogenous nucleic acid is transferred transiently into a cell, for example a mammalian cell; while “transduced” means a exogenous nucleic acid is transferred permanently into a cell, often a mammalian cell, for example by viruses or viral vectors; and “transformed” means a exogenous nucleic acid is transferred into a cell, often bacterial or yeast cells.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

Direct in vivo high-throughput mutational analysis of functional cancer drivers in the mammalian brain has been difficult, due to the nature of biological complexity in vivo. it is challenging to perform high-throughput analysis of mutants in autochthonous models of cancer; that is, models in which the tumors directly evolve from normal cells at the organ site in situ in immunocompetent mice and without cellular transplantation.

As demonstrated herein, this challenge was overcome by developing a focused AAV-CRISPR library with AAV encoding functional elements and sgRNA pools targeting a top pan-cancer putative TSG set, in combination with the conditional LSL-Cas9 transgenic mice. Thus, a powerful platform was developed to perform high efficiency AAV-CRISPR mediated pooled mutagenesis for direct in vivo analysis of many genes in mice. This platform and approach of AAV-CRISPR mediated pooled mutagenesis in conjunction with targeted capture sequencing provides an efficient platform for massively parallel analysis of GBM drivers directly in vivo.

### Methods

In one aspect, the invention includes a method of determining a treatment for glioblastoma in a subject. The method comprises contacting a plurality of Adeno-Associated Virus-Clustered Regularly Interspaced Short Palidromic Repeats (AAV-CRISPR) vectors with a sample from the subject. The vectors comprise Cas9 and a plurality of nucleotide sequences homologous to a plurality of tumor suppressor genes (TSGs). Nucleic acids are isolated from the sample and sequenced. The sequencing data are analyzed identifying at least one mutation which determines the treatment for glioblastoma in the subject.

The mutations claimed herein can be any combination of insertions or deletions, including but not limited to a single base insertion, a single base deletion, a frameshift, a rearrangement, and an insertion or deletion of 2, 3, 4, 5, 6, 7, 8, 9, 10, 15, 20, 25, 30, 40, 50, 60, 70, 80, 90, 100, 150, 200, 250, 300, any and all numbers in between, bases. The mutation can occur in a gene or in a non-coding region. The location of the mutation can provide information as to the type of treatment needed. In a non-limiting example, if a mutation occurs in a specific gene rendering that gene non-functional, a drug that acts on that particular gene will not be considered for treatment. In a non-limiting example, if a drug is known to act on a particular gene and that gene is not mutated, that drug will be considered for treatment.

The invention also includes a method of determining at least one glioblastoma driver mutation in a subject's sample. The method comprises contacting an AAV-CRISPR library with a subject's sample, wherein the vectors comprise Cas9 and a plurality of nucleotide sequences homologous to a plurality of tumor suppressor genes (TSGs). Then nucleic acids are isolated from the sample and sequenced. The sequencing data is analyzed thus determining the at least one glioblastoma driver mutation.

In certain embodiments of the invention, the plurality of nucleotide sequences homologous to a plurality of TSGs comprises SEQ ID NOs. 1-280.

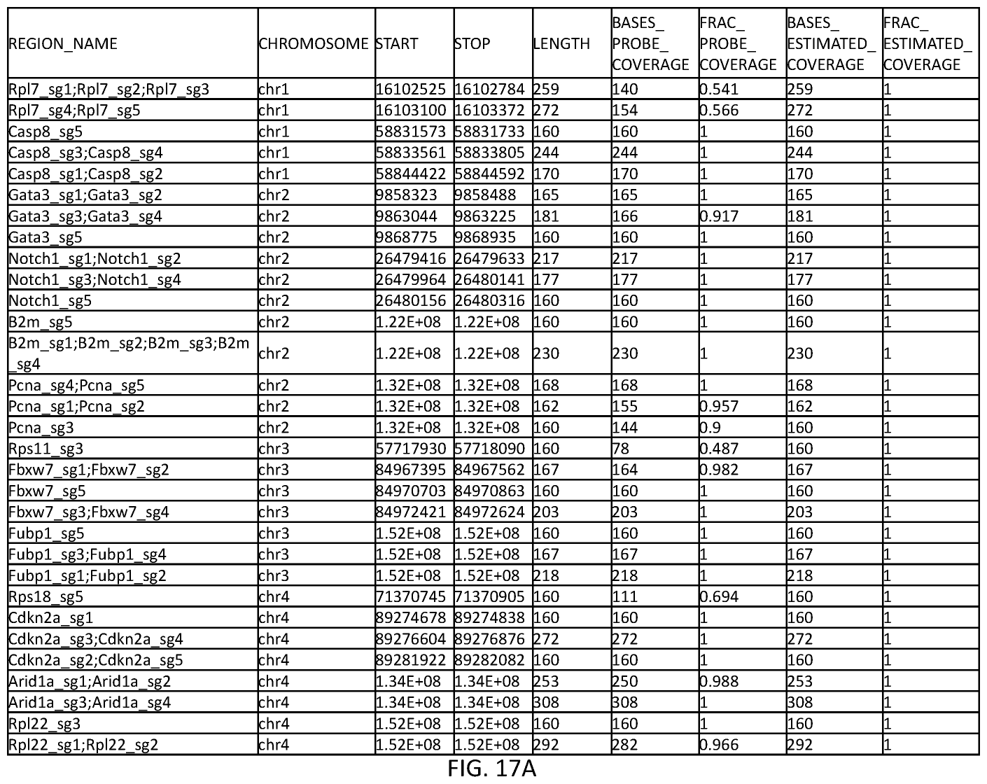

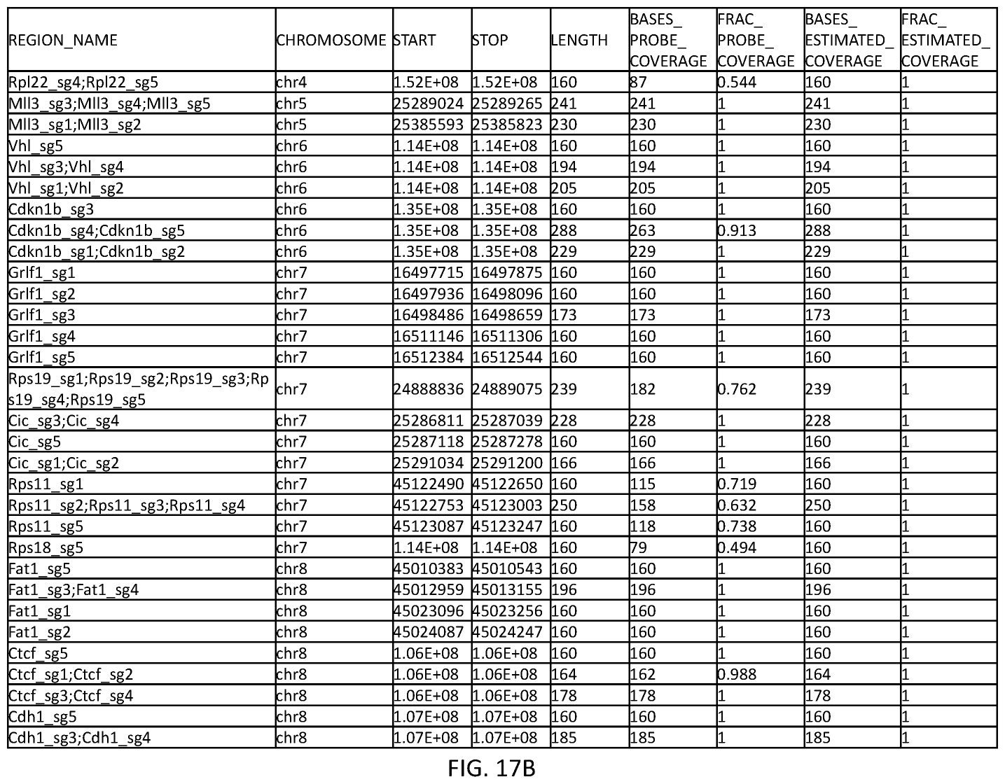

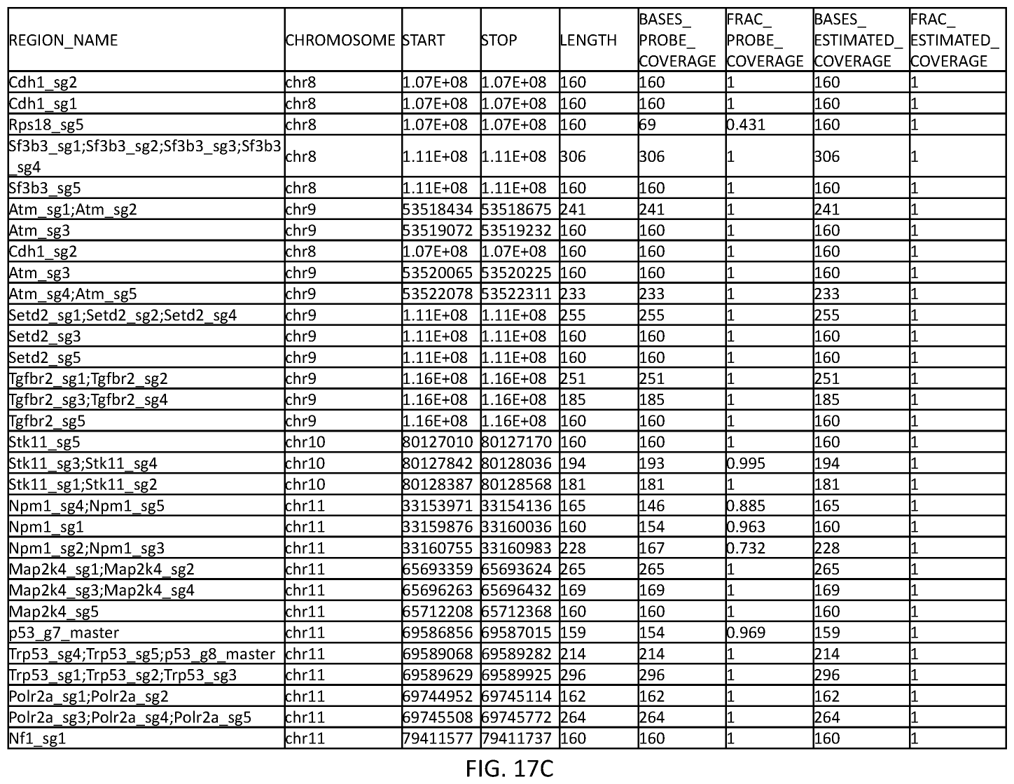

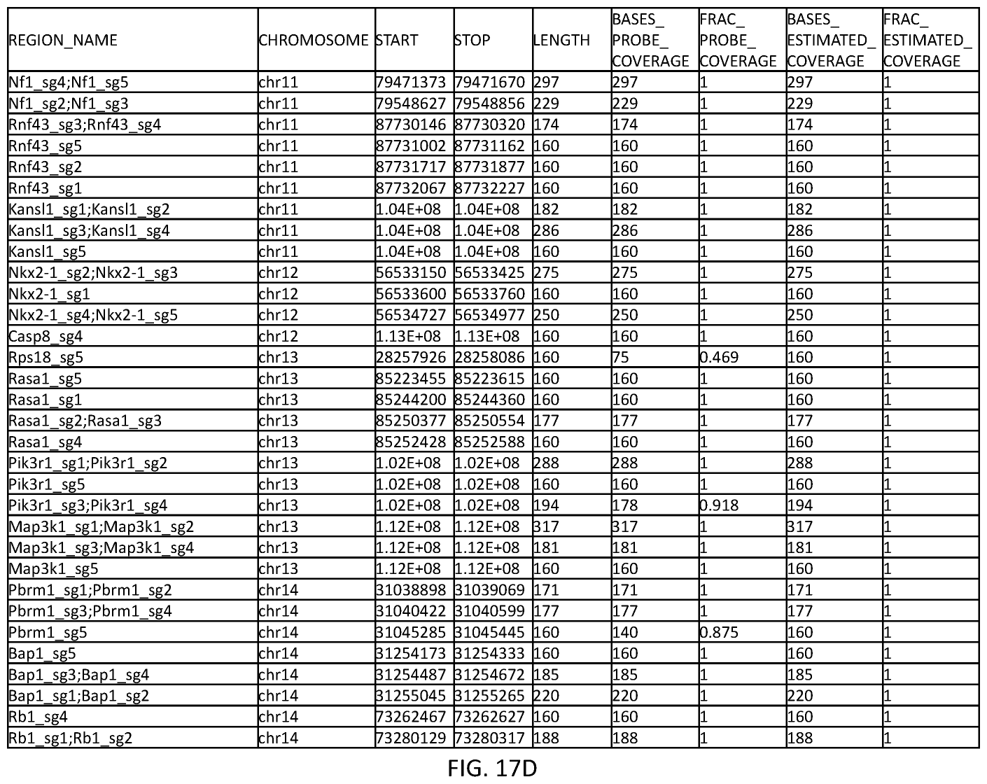

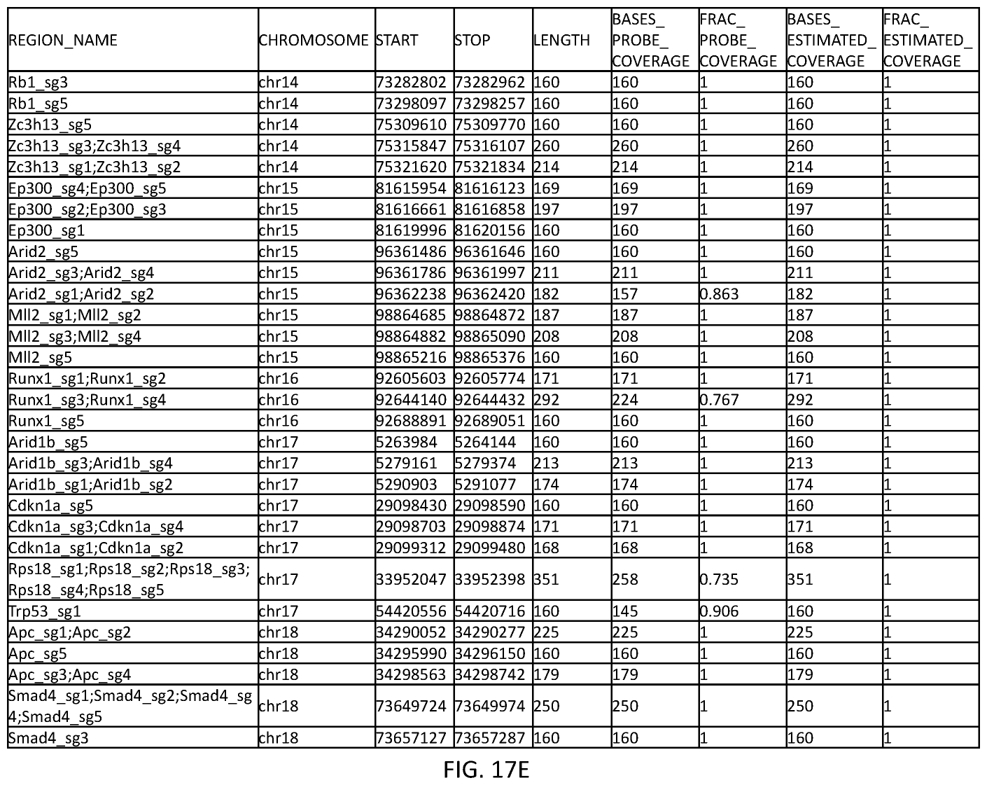

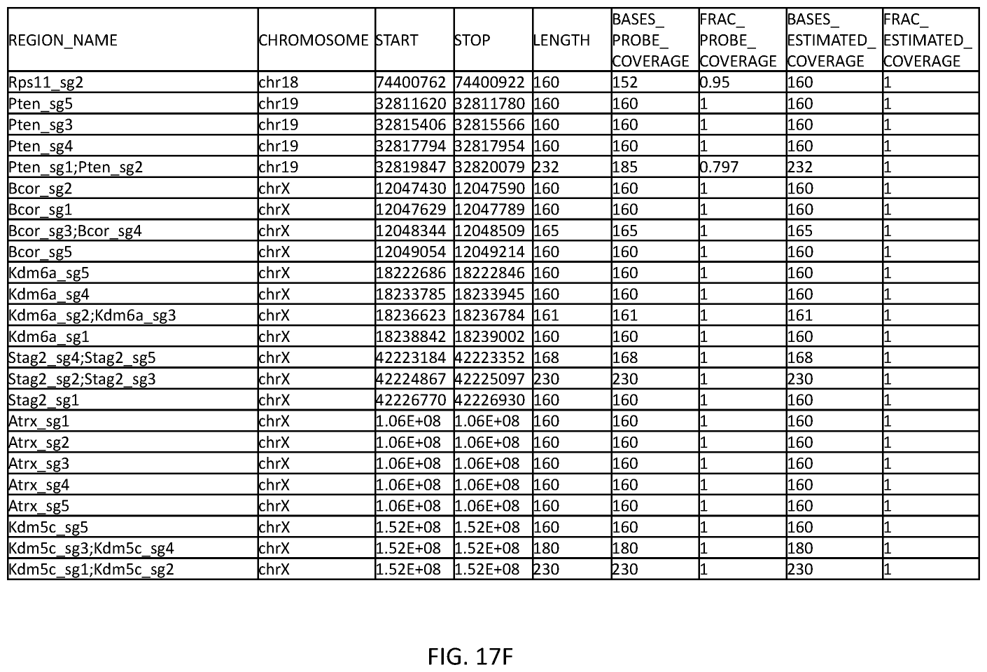

Nucleotide sequencing or ‘sequencing’, as it is commonly known in the art, can be performed by standard methods commonly known to one of ordinary skill in the art. In certain embodiments of the invention, sequencing is performed by targeted capture sequencing. Targeted captured sequencing can be performed as described herein, or by methods commonly performed by one of ordinary skill in the art. In one non-limiting example, targeted capture sequencing is performed using the target capture probes detailed herein and in FIGS. 17A-17F.

In certain embodiments of the invention, the sample is a plurality of glioma cells. In other embodiments, the sample is a tumor. In yet other embodiments, the tumor is a glioblastoma. In yet other embodiments, the sample is a non-tumor or non-cancerous cell. For example, the sample can be a non-cancerous cell or cell line that is administered an AAV-CRISPR vector and monitored for excessive proliferation. The cells are subsequently sequenced to determine the driver mutations. The driver mutations can be in TSGs or in other genes.

In one aspect, the invention includes a method of determining at least one glioblastoma driver mutation in vivo. The method comprises selecting in silico nucleotide sequences from a plurality of tumor suppressor genes (TSGs) and designing in silico a plurality of short guide RNAs (sgRNAs) homologous to the plurality of TSGs. Then a plurality of oligonucleotides are synthesized according to the sgRNAs designed. The oligonucleotides are introduced into a plurality of AAV-CRISPR vectors that contain Cas9. The plurality AAV-CRISPR vectors are administered to the brain of an animal. A tumor is isolated from the animal and nucleic acids are isolated from the tumor and sequenced. The data sequencing are analyzed identifying at least one glioblastoma driver mutation.

In certain embodiments of the invention, the animal is a mouse. Other animals that can be used include but are not limited to rats, rabbits, dogs, cats, horses, pigs, cows and birds. In certain embodiments, the animal is a human. The AAV-CRISPR vectors can be administered to an animal by any means standard in the art. For example the vectors can be injected into the animal. The injections can be intravenous, subcutaneous, intraperitoneal, or directly into a tissue or organ.

The AAV-CRISPR vector can include additional components. In one embodiment, the AAV-CRISPR vector is comprised of the components as described herein. The AAV-CRISPR can also include (1) an astrocyte-specific GFAP promoter, for example a polII promoter, (2) a constitutive U6 polIII promoter, (3) sgRNA spacer cloning site with double SapI type II restriction enzyme cutting site; (4) an sgRNA backbone derived from an 89 bp chimeric backbone from Streptococcus pyogenes Cas9 tracrRNA; and (5) a Cre recombinase.

In one aspect, the invention includes a method for determining the driver mutations responsible for a cancer. The method comprises selecting candidate tumor suppressor genes (TSGs) associated with the cancer and designing short guide RNAs (sgRNAs) against those TSGs, The sgRNAs are synthesized as oligonucleotides and cloned into an AAV-CRISPR vector generating a library packaged in the vectors. The vectors are propagated in cells, isolated (i.e., purified from the cells), and injected into mice. Mice are monitored for tumor formation, survival, histopathology, and pathology. Tumors are isolated from the mice and nucleic acids (and/or proteins) are extracted from the tumors. Targeted sequencing capture probes are designed against the TSGs and used to sequence the nucleic acids from the cancer samples/hybridized to the nucleic acids from the cancer samples and sequenced. The sequencing data are analyzed to determine the mutations (indels—insertions and deletions) in the TSGs, thus determining the drivers of the cancer.

### Compositions

In one aspect, the invention includes an AAV-CRISPR library. The AAV-CRISPR library comprises a plurality of AAV vectors comprising: Cas9 and a plurality of nucleic acids homologous to a plurality of Tumor Suppressor Gene (TSGs). In certain embodiments, the plurality of nucleic acids comprises SEQ ID NOs 1-280.

The invention also includes a kit for determining at least one driver mutation in a glioblastoma sample. The kit comprises an AAV-CRISPR library comprising a plurality of AAV vectors comprising Cas9 and a plurality of nucleic acids homologous to a plurality of Tumor Suppressor Gene (TSGs). In one embodiment, the plurality of nucleic acids comprise SEQ ID NOs. 1-280. The kits also include instructional material for use thereof. Instructional material can include directions for using the components of the kit as well as instructions or guidance for interpreting the results. For example the instructional material can include instructions for determining driver mutations in a glioblastoma sample.

Another aspect of the invention includes an AAV-CRISPR vector containing a cassette that expresses Cre recombinase under the control of a GFAP promoter for conditional induction of Cas9 expression and two sgRNA cassettes: one encoding an sgRNA targeting Trp53, and the other an open sgRNA cassette. In one aspect, the invention includes a vector comprising SEQ ID NO: 289. In another aspect, the invention includes a kit comprising a vector comprising SEQ ID NO: 289 and instructional material for use thereof.

### CRISPR/Cas9

The CRISPR/Cas9 system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and CAR T cells. The CRISPR/Cas9 system can simultaneously target multiple genomic loci by co-expressing a single Cas9 protein with two or more gRNAs, making this system uniquely suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The Red domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US20140068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, an pAd5F35-CRISPR vector. In other embodiments, the Cas expression vector induces expression of Cas9 endonuclease. Other endonucleases may also be used, including but not limited to, T7, Cas3, Cas8a, Cas8b, Cas10d, Cse1, Csy1, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). However, it should be appreciated that other inducible promoters can be used. The inducing agent can be a selective condition (e.g., exposure to an agent, for example an antibiotic) that results in induction of the inducible promoter. This results in expression of the Cas expression vector.

In certain embodiments, guide RNA(s) and Cas9 can be delivered to a cell as a ribonucleoprotein (RNP) complex. RNPs are comprised of purified Cas9 protein complexed with gRNA and are well known in the art to be efficiently delivered to multiple types of cells, including but not limited to stem cells and immune cells (Addgene, Cambridge, Mass., Mirus Bio LLC, Madison, Wis.).

The guide RNA is specific for a genomic region of interest and targets that region for Cas endonuclease-induced double strand breaks. The target sequence of the guide RNA sequence may be within a loci of a gene or within a non-coding region of the genome. In certain embodiments, the guide nucleic acid sequence is at least 10, 11, 12, 13, 14, 15, 16, 17, 18, 19, 20, 21, 22, 23, 24, 25, 26, 27, 30, 31, 32, 33, 34, 35, 36, 37, 38, 39, 40 or more nucleotides in length.

Guide RNA (gRNA), also referred to as “short guide RNA” or “sgRNA”, provides both targeting specificity and scaffolding/binding ability for the Cas9 nuclease. The gRNA can be a synthetic RNA composed of a targeting sequence and scaffold sequence derived from endogenous bacterial crRNA and tracrRNA. gRNA is used to target Cas9 to a specific genomic locus in genome engineering experiments. Guide RNAs can be designed using standard tools well known in the art.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of an endogenous CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In certain embodiments, the CRISPR/Cas is derived from a type II CRISPR/Cas system. In other embodiments, the CRISPR/Cas system is derived from a Cas9 protein. The Cas9 protein can be from Streptococcus pyogenes, Streptococcus thermophilus, or other species.

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the present invention. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4^{th} Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

### Introduction of Nucleic Acids

Methods of introducing nucleic acids into a cell include physical, biological and chemical methods. Physical methods for introducing a polynucleotide, such as DNA or RNA, into a cell include transfection, transformation, transduction, calcium phosphate precipitation, lipofection, particle bombardment, microinjection, electroporation, and the like. RNA and DNA can be introduced into cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany). RNA and DNA can also be introduced into cells using cationic liposome mediated transfection using lipofection, using polymer encapsulation, using peptide mediated transfection, or using biolistic particle delivery systems such as “gene guns” (see, for example, Nishikawa, et al. Hum Gene Ther., 12(8):861-70 (2001).

Biological methods for introducing a polynucleotide of interest into a cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362. Non-viral vector such as plasmids can also be used to introduce nucleic acids or polynucleotides into a cell. In certain embodiments plasmids containing guide RNAs are transfected into a cell.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell, in order to confirm the presence of the nucleic acids in the host cell, a variety of assays may be performed. Such assays include, for example, “molecular biological” assays well known to those of skill in the art, such as gel electrophoresis, Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

It should be understood that the method and compositions that would be useful in the present invention are not limited to the particular formulations set forth in the examples. The following examples are put forth so as to provide those of ordinary skill in the art with a complete disclosure and description, and are not intended to limit the scope of what the inventors regard as their invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, Molecular Cloning: A Laboratory Manual”, fourth edition (Sambrook et al. (2012) Molecular Cloning, Cold Spring Harbor Laboratory); “Oligonucleotide Synthesis” (Gait, M. J. (1984). Oligonucleotide synthesis. IRL press); “Culture of Animal Cells” (Freshney, R. (2010). Culture of animal cells. Cell Proliferation, 15(2.3), 1); “Methods in Enzymology” “Weir's Handbook of Experimental Immunology” (Wiley-Blackwell; 5 edition (Jan. 15, 1996); “Gene Transfer Vectors for Mammalian Cells” (Miller and Carlos, (1987) Cold Spring Harbor Laboratory, New York); “Short Protocols in Molecular Biology” (Ausubel et al., Current Protocols; 5 edition (Nov. 5, 2002)); “Polymerase Chain Reaction: Principles, Applications and Troubleshooting”, (Babar, M., VDM Verlag Dr. Müler (Aug. 17, 2011)); “Current Protocols in Immunology” (Coligan, John Wiley & Sons, Inc. Nov. 1, 2002).

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

The materials and methods employed in these experiments are now described.

Design, synthesis and cloning of the mTSG library: Due to cellular complexity in the organs in vivo, a small focused library was designed for viral transduction. Briefly, pan-cancer mutation data from 15 cancer types were retrieved from The Cancer Genome Atlas (TCGA portal) via cBioPortal (Gao et al., 2013, Sci Signal 6) and Synapse (www dot synapse dot org). Significantly mutated genes were calculated similar to previously described methods (Davoli et al., 2013, Cell 155, 948-962; Kandoth et al., 2013, Nature 502, 333-339; Lawrence et al., 2014, Nature 505, 495-501; Lawrence et al., 2013, Nature 499, 214-218). Known oncogenes were excluded and only known or predicted tumor suppressor genes (TSGs) were included. The top 50 TSGs were chosen, and their mouse homologs (mTSG) were retrieved from mouse genome informatics (MGI) (www dot informatics dot jax dot org). A total of 49 mTSGs were found. A total of 7 known housekeeping genes were chosen as internal controls. SgRNAs were designed against these 56 genes (Shalem et al., 2014, Science 343, 84-87; Wang et al., 2014, Science 343, 80-84) with custom scripts. Five sgRNAs were chosen for each gene, plus 8 non-targeting controls (NTCs), making a total 288 sgRNAs in the mTSG library. There were two sets of duplicate sgRNAs, Cdkn2a-sg2/Cdkn2a-sg5, and Rp122-sg4/Rp122-sg5, leaving a total of 286 unique sgRNAs.

Design, cloning of an AAV-CRISPR GBM vector and mTSG sgRNA library cloning: An AAV-CRISPR vector was designed for astrocyte-specific genome editing. This vector contained a cassette that specifically expresses Cre recombinase under the control of a GFAP promoter for conditional induction of Cas9 expression in astrocytes in the brain when delivered to LSL-Cas9 mice (Platt et al., 2014, Cell 159, 440-455). Two sgRNA cassettes were built in this vector, one encoding an sgRNA targeting Trp53, the guardian of the genome and most frequently mutated gene in cancer (Davoli et al., 2013, Cell 155, 948-962; Kandoth et al., 2013, Nature 502, 333-339; Lawrence et al., 2014, Nature 505, 495-501), with the other being an open sgRNA cassette (double SapI sites for sgRNA cloning) enabling flexible targeting of genes of interest in either individual or pooled manner. The vector was generated by gBlock gene fragment synthesis (IDT) followed by Gibson assembly (NEB). The mTSG library was generated by oligo synthesis, pooled, and cloned into the double SapI sites of the AAV-CRISPR GBM vector. The library cloning was done at over 100× coverage to ensure proper representation. Plasmid library representation was readout by barcoded Illumina sequencing (Chen et al., 2015, Cell 160, 1246-1260) with primers customized to this vector.

| Vector pAAV-sgRNA-GFAP-Cre: | |
|---|---|
| (SEQ ID NO: 289) | |
| 1 | cctgcaggca gctgcgcgct cgctcgctca ctgaggccgc ccgggcaaag cccgggcgtc |
| 61 | gggcgacctt tggtcgcccg gcctcagtga gcgagcgagc gcgcagagag ggagtggcca |
| 121 | actccatcac taggggttcc tgcggccgca cgcgtgaggg cctatttccc atgattcctt |
| 181 | catatttgca tatacgatac aaggctgtta gagagataat tggaattaat ttgactgtaa |
| 241 | acacaaagat attagtacaa aatacgtgac gtagaaagta ataatttctt gggtagtttg |
| 301 | cagttttaaa attatgtttt aaaatggact atcatatgct taccgtaact tgaaagtatt |
| 361 | tcgatttctt ggctttatat atcttGTGGA AAGGACGAAA CACCGTGTAA TAGCTCCTGC |
| 421 | ATGGgtttta gagctaGAAA tagcaagtta aaataaggct agtccgttat caacttgaaa |
| 481 | aagtggcacc gagtcggtgc TTTTTTtcta gaagagggcc tatttcccat gattccttca |
| 541 | tatttgcata tacgatacaa ggctgttaga gagataattg gaattaattt gactgtaaac |
| 601 | acaaagatat tagtacaaaa tacgtgacgt agaaagtaat aatttcttgg gtagtttgca |
| 661 | gttttaaaat tatgttttaa aatggactat catatgctta ccgtaacttg aaagtatttc |
| 721 | gatttcttgg ctttatatat cttGTGGAAA GGACGAAACA CCggaagagc gagctcttct |
| 781 | gttttagagc taGAAAtagc aagttaaaat aaggctagtc cgttatcaac ttgaaaaagt |
| 841 | ggcaccgagt cggtgcTTTT TTggtaccag gatcccacct ccctctctgt gctgggactc |
| 901 | acagagggag acctcaggag gcagtctgtc catcacatgt ccaaatgcag agcataccct |
| 961 | gggctgggcg cagtggcgca caactgtaat tccagcactt tgggaggctg atgtggaagg |
| 1021 | atcacttgag cccagaagtt ctagaccagc ctgggcaaca tggcaagacc ctatctctac |
| 1081 | aaaaaaagtt aaaaaatcag ccacgtgtgg tgacacacac ctgtagtccc agctattcag |
| 1141 | gaggctgagg tgaggggatc acttaaggct gggaggttga ggctgcagtg agtcgtggtt |
| 1201 | gcgccactgc actccagcct gggcaacagt gagaccctgt ctcaaaagac aaaaaaaaaa |
| 1261 | aaaaaaaaaa aaagaacata tcctggtgtg gagtagggga cgctgctctg acagaggctc |
| 1321 | gggggcctga gctggctctg tgagctgggg aggaggcaga cagccaggcc ttgtctgcaa |
| 1381 | gcagacctgg cagcattggg ctggccgccc cccagggcct cctcttcatg cccagtgaat |
| 1441 | gactcacctt ggcacagaca caatgttcgg ggtgggcaca gtgcctgctt cccgccgcac |
| 1501 | cccagccccc ctcaaatgcc ttccgagaag cccattgagc agggggcttg cattgcaccc |
| 1561 | cagcctgaca gcctggcatc ttgggataaa agcagcacag ccccctaggg gctgcccttg |
| 1621 | ctgtgtggcg ccaccggcgg tggagaacaa ggctctattc agcctgtgcc caggaaaggg |
| 1681 | gatcagggga tgcccaggca tggacagtgg gtggcagggg gggagaggag ggctgtctgc |
| 1741 | ttcccagaag tccaaggaca caaatgggtg aggggactgg gcagggttct gaccctgtgg |
| 1801 | gaccagagtg gagggcgtag atggacctga agtctccagg gacaacaggg cccaggtctc |
| 1861 | aggctcctag ttgggcccag tggctccagc gtttccaaac ccatccatcc ccagaggttc |
| 1921 | ttcccatctc tccaggctga tgtgtgggaa ctcgaggaaa taaatctcca gtgggagacg |
| 1981 | gaggggtggc cagggaaacg gggcgctgca ggaataaaga cgagccagca cagccagctc |
| 2041 | atgtgtaacg gctttgtgga gctgtcaagg cctggtctct gggagagagg cacagggagg |
| 2101 | ccagacaagg aaggggtgac ctggagggac agatccaggg gctaaagtcc tgataaggca |
| 2161 | agagagtgcc ggccccctct tgccctatca ggacctccac tgccacatag aggccatgat |
| 2221 | tgacccttag acaaagggct ggtgtccaat cccagccccc agccccagaa ctccagggaa |
| 2281 | tgaatgggca gagagcagga atgtgggaca tctgtgttca agggaaggac tccaggagtc |
| 2341 | tgctgggaat gaggcctagt aggaaatgag gtggcccttg agggtacaga acaggttcat |
| 2401 | tcttcgccaa attcccagca ccttgcaggc acttacagct gagtgagata atgcctgggt |
| 2461 | tatgaaatca aaaagttgga aagcaggtca gaggtcatct ggtacagccc ttccttccct |
| 2521 | tttttttttt ttttttttgt gagacaaggt ctctctctgt tgcccaggct ggagtggcgc |
| 2581 | aaacacagct cactgcagcc tcaacctact gggctcaagc aatcctccag cctcagcctc |
| 2641 | ccaaagtgct gggattacaa gcatgagcca ccccactcag ccctttcctt cctttttaat |
| 2701 | tgatgcataa taattgtaag tattcatcat ggtccaacca accctttctt gacccacctt |
| 2761 | cctagagaga gggtcctctt gcttcagcgg tcagggcccc agacccatgg tctggctcca |
| 2821 | ggtaccacct gcctcatgca ggagttggcg tgcccaggaa gctctgcctc tgggcacagt |
| 2881 | gacctcagtg gggtgagggg agctctcccc atagctgggc tgcggcccaa ccccaccccc |
| 2941 | tcaggctatg ccagggggtg ttgccagggg cacccgggca tcgccagtct agcccactcc |
| 3001 | ttcataaagc cctcgcatcc caggagcgag cagagccaga gcaggatgga gaggagacgc |
| 3061 | atcacctccg ctgctcgccg ggcgtacggc caccatgccc aagaagaaga ggaaggtgtc |
| 3121 | caatctcctg actgttcacc agaacctccc tgcgctgcca gtagatgcca ctagcgatga |
| 3181 | ggtcaggaaa aatctcatgg atatgtttag ggatagacag gcgttttctg aacacacctg |
| 3241 | gaaaatgctg cttagcgtgt gccgatcctg ggcagcctgg tgtaagctga acaatcgcaa |
| 3301 | atggttcccc gccgagccgg aggacgtgcg cgattacctg ctgtatctcc aggcaagagg |
| 3361 | gctggctgtc aagactatcc agcagcactt gggccaactg aatatgctgc atcgacgcag |
| 3421 | cgggctcccc cggcctagcg attcaaacgc agtctccctt gttatgagga gaattagaaa |
| 3481 | ggaaaacgta gatgcgggtg agagggctaa gcaggctctc gcttttgagc ggactgattt |
| 3541 | cgaccaggtc agatccctga tggagaacag cgatcggtgc caggacatca ggaacctcgc |
| 3601 | atttctggga attgcatata acacacttct gcgcatagct gagatcgccc ggatcagagt |
| 3661 | gaaagacatc agtcgaacgg acggcggccg gatgcttatt catattggac gcacaaagac |
| 3721 | attggtcagc accgctggcg ttgaaaaggc cttgtccctg ggcgtaacga agctggtgga |
| 3781 | aagatggatc tcagtgtccg gcgtggctga cgaccctaat aattacttgt tctgtcgagt |
| 3841 | gagaaaaaac ggagtcgccg cgccctctgc caccagccaa ttgagtacac gggcccttga |
| 3901 | agggatcttt gaggcaaccc accgactcat atacggagcc aaggatgaca gtggccagag |
| 3961 | gtatctcgcc tggtcaggtc attctgctag ggtgggggcc gcacgagaca tggcgcgggc |
| 4021 | aggagtctcc ataccagaga ttatgcaagc tggaggttgg acaaatgtga acatcgttat |
| 4081 | gaactatatc cgcaatcttg actctgaaac cggggccatg gtgagactgc tcgaagatgg |
| 4141 | tgactaccca tacgatgttc cagattacgc tTAAGAATTC gatatcaagc ttAATAAAAG |
| 4201 | ATCTTTATTT TCATTAGATC TGTGTGTTGG TTTTTTGTGT ggtaaccacg tgcggaccga |
| 4261 | gcggccgcag gaacccctag tgatggagtt ggccactccc tctctgcgcg ctcgctcgct |
| 4321 | cactgaggcc gggcgaccaa aggtcgcccg acgcccgggc tttgcccggg cggcctcagt |
| 4381 | gagcgagcga gcgcgcagct gcctgcaggg gcgcctgatg cggtattttc tccttacgca |
| 4441 | tctgtgcggt atttcacacc gcatacgtca aagcaaccat agtacgcgcc ctgtagcggc |
| 4501 | gcattaagcg cggcgggtgt ggtggttacg cgcagcgtga ccgctacact tgccagcgcc |
| 4561 | ctagcgcccg ctcctttcgc tttcttccct tcctttctcg ccacgttcgc cggctttccc |
| 4621 | cgtcaagctc taaatcgggg gctcccttta gggttccgat ttagtgcttt acggcacctc |
| 4681 | gaccccaaaa aacttgattt gggtgatggt tcacgtagtg ggccatcgcc ctgatagacg |
| 4741 | gtttttcgcc ctttgacgtt ggagtccacg ttctttaata gtggactctt gttccaaact |
| 4801 | ggaacaacac tcaaccctat ctcgggctat tcttttgatt tataagggat tttgccgatt |
| 4861 | tcggcctatt ggttaaaaaa tgagctgatt taacaaaaat ttaacgcgaa ttttaacaaa |
| 4921 | atattaacgt ttacaatttt atggtgcact ctcagtacaa tctgctctga tgccgcatag |
| 4981 | ttaagccagc cccgacaccc gccaacaccc gctgacgcgc cctgacgggc ttgtctgctc |
| 5041 | ccggcatccg cttacagaca agctgtgacc gtctccggga gctgcatgtg tcagaggttt |
| 5101 | tcaccgtcat caccgaaacg cgcgagacga aagggcctcg tgatacgcct atttttatag |
| 5161 | gttaatgtca tgataataat ggtttcttag acgtcaggtg gcacttttcg gggaaatgtg |
| 5221 | cgcggaaccc ctatttgttt atttttctaa atacattcaa atatgtatcc gctcatgaga |
| 5281 | caataaccct gataaatgct tcaataatat tgaaaaagga agagtatgag tattcaacat |
| 5341 | ttccgtgtcg cccttattcc cttttttgcg gcattttgcc ttcctgtttt tgctcaccca |
| 5401 | gaaacgctgg tgaaagtaaa agatgctgaa gatcagttgg gtgcacgagt gggttacatc |
| 5461 | gaactggatc tcaacagcgg taagatcctt gagagttttc gccccgaaga acgttttcca |
| 5521 | atgatgagca cttttaaagt tctgctatgt ggcgcggtat tatcccgtat tgacgccggg |
| 5581 | caagagcaac tcggtcgccg catacactat tctcagaatg acttggttga gtactcacca |
| 5641 | gtcacagaaa agcatcttac ggatggcatg acagtaagag aattatgcag tgctgccata |
| 5701 | accatgagtg ataacactgc ggccaactta cttctgacaa cgatcggagg accgaaggag |
| 5761 | ctaaccgctt ttttgcacaa catgggggat catgtaactc gccttgatcg ttgggaaccg |
| 5821 | gagctgaatg aagccatacc aaacgacgag cgtgacacca cgatgcctgt agcaatggca |
| 5881 | acaacgttgc gcaaactatt aactggcgaa ctacttactc tagcttcccg gcaacaatta |
| 5941 | atagactgga tggaggcgga taaagttgca ggaccacttc tgcgctcggc ccttccggct |
| 6001 | ggctggttta ttgctgataa atctggagcc ggtgagcgtg ggtctcgcgg tatcattgca |
| 6061 | gcactggggc cagatggtaa gccctcccgt atcgtagtta tctacacgac ggggagtcag |
| 6121 | gcaactatgg atgaacgaaa tagacagatc gctgagatag gtgcctcact gattaagcat |
| 6181 | tggtaactgt cagaccaagt ttactcatat atactttaga ttgatttaaa acttcatttt |
| 6241 | taatttaaaa ggatctaggt gaagatcctt tttgataatc tcatgaccaa aatcccttaa |
| 6301 | cgtgagtttt cgttccactg agcgtcagac cccgtagaaa agatcaaagg atcttcttga |
| 6361 | gatccttttt ttctgcgcgt aatctgctgc ttgcaaacaa aaaaaccacc gctaccagcg |
| 6421 | gtggtttgtt tgccggatca agagctacca actctttttc cgaaggtaac tggcttcagc |
| 6481 | agagcgcaga taccaaatac tgtccttcta gtgtagccgt agttaggcca ccacttcaag |
| 6541 | aactctgtag caccgcctac atacctcgct ctgctaatcc tgttaccagt ggctgctgcc |
| 6601 | agtggcgata agtcgtgtct taccgggttg gactcaagac gatagttacc ggataaggcg |
| 6661 | cagcggtcgg gctgaacggg gggttcgtgc acacagccca gcttggagcg aacgacctac |
| 6721 | accgaactga gatacctaca gcgtgagcta tgagaaagcg ccacgcttcc cgaagggaga |
| 6781 | aaggcggaca ggtatccggt aagcggcagg gtcggaacag gagagcgcac gagggagctt |
| 6841 | ccagggggaa acgcctggta tctttatagt cctgtcgggt ttcgccacct ctgacttgag |
| 6901 | cgtcgatttt tgtgatgctc gtcagggggg cggagcctat ggaaaaacgc cagcaacgcg |
| 6961 | gcctttttac ggttcctggc cttttgctgg ccttttgctc acatgt |

GFAP: (Genbank M67446.1 Human glial fibrillary acidic protein (GFAP), exon 1)
(SEQ ID NO: 290)
cccacctccctctctgtgctgggactcacagagggagacctcaggaggcagtctgtccatcacatgtccaaatgcaga
gcataccctgggctgggcgcagtggcgcacaactgtaattccagcactttgggaggctgatgtggaaggatcacttga
gcccagaagttctagaccagcctgggcaacatggcaagaccctatctctacaaaaaaagttaaaaaatcagccacgtg
tggtgacacacacctgtagtcccagctattcaggaggctgaggtgaggggatcacttaaggctgggaggttgaggctg
cagtgagtcgtggttgcgccactgcactccagcctgggcaacagtgagaccctgtctcaaaagacaaaaaaaaaaaaa
aaaaaaaaaagaacatatcctggtgtggagtaggggacgctgctctgacagaggctcgggggcctgagctggctctgt
gagctggggaggaggcagacagccaggccttgtctgcaagcagacctggcagcattgggctggccgccccccagggcc
tcctcttcatgcccagtgaatgactcaccttggcacagacacaatgttcggggtgggcacagtgcctgcttcccgccg
caccccagcccccctcaaatgccttccgagaagcccattgagcagggggcttgcattgcaccccagcctgacagcctg
gcatcttgggataaaagcagcacagccccctaggggctgcccttgctgtgtggcgccaccggcggtggagaacaaggc
tctattcagcctgtgcccaggaaaggggatcaggggatgcccaggcatggacagtgggtggcagggggggagaggagg
gctgtctgcttcccagaagtccaaggacacaaatgggtgaggggactgggcagggttctgaccctgtgggaccagagt
ggagggcgtagatggacctgaagtctccagggacaacagggcccaggtctcaggctcctagttgggcccagtggctcc
agcgtttccaaacccatccatccccagaggttcttcccatctctccaggctgatgtgtgggaactcgaggaaataaat
ctccagtgggagacggaggggtggccagggaaacggggcgctgcaggaataaagacgagccagcacagccagctcatg
tgtaacggctttgtggagctgtcaaggcctggtctctgggagagaggcacagggaggccagacaaggaaggggtgacc
tggagggacagatccaggggctaaagtcctgataaggcaagagagtgccggccccctcttgccctatcaggacctcca
ctgccacatagaggccatgattgacccttagacaaagggctggtgtccaatcccagcccccagccccagaactccagg
gaatgaatgggcagagagcaggaatgtgggacatctgtgttcaagggaaggactccaggagtctgctgggaatgaggc
ctagtaggaaatgaggtggcccttgagggtacagaacaggttcattcttcgccaaattcccagcaccttgcaggcact
tacagctgagtgagataatgcctgggttatgaaatcaaaaagttggaaagcaggtcagaggtcatctggtacagccct
tccttccctttttttttttttttttttgtgagacaaggtctctctctgttgcccaggctggagtggcgcaaacacagc
tcactgcagcctcaacctactgggctcaagcaatcctccagcctcagcctcccaaagtgctgggattacaagcatgag
ccaccccactcagccctttccttcctttttaattgatgcataataattgtaagtattcatcatggtccaaccaaccct
ttcttgacccaccttcctagagagagggtcctcttgcttcagcggtcagggccccagacccatggtctggctccaggt
accacctgcctcatgcaggagttggcgtgcccaggaagctctgcctctgggcacagtgacctcagtggggtgagggga
gctctccccatagctgggctgcggcccaaccccaccccctcaggctatgccagggggtgttgccaggggcacccgggc
atcgccagtctagcccactccttcataaagccctcgcatcccaggagcgagcagagccagagcaggatggagaggaga
cgcatcacctccgctgctcgccggg

AAV-mTSG viral library production: The AAV-CRISPR GBM plasmid vector (AAV-vector) and library (AAV-mTSG) were subjected to AAV9 production and chemical purification. Briefly, HEK 293FT cells (ThermoFisher) were transiently transfected with transfer (AAV-vector or AAV-mTSG) serotype (AAV9) and packaging (pDF6) plasmids using polyethyleneimine (PEI). Each replicate consisted of five 15-cm tissue culture dishes or T-175 flasks (Corning) of 80% confluent HEK 293FT cells. Multiple replicates were pooled to enhance production yield. Approximately 72 hours post transfection, cells were dislodged and transferred to a conical tube in sterile PBS. 1/10 volume of pure chloroform was added and the mixture was incubated at 37° C. and vigorously shaken for 1 hour. NaCl was added to a final concentration of 1 M and the mixture was shaken until dissolved and then pelleted at 20 k g at 4° C. for 15 minutes. The aqueous layer was discarded while the chloroform layer was transferred to another tube. PEG8000 was added to 10% (w/v) and shaken until dissolved. The mixture was incubated at 4° C. for 1 hour and then spun at 20 k g at 4° C. for 15 minutes. The supernatant was discarded, and the pellet was resuspended in DPBS plus MgCl₂, treated with Benzonase (Sigma), and incubated at 37° C. for 30 minutes. Chloroform (1:1 volume) was then added, shaken, and spun down at 12 k g at 4 C for 15 min. The aqueous layer was isolated and passed through a 100 kDa MWCO (Millipore). The concentrated solution was washed with PBS and the filtration process was repeated. Virus was titered by qPCR using custom Taqman assays (ThermoFisher) targeted to Cre.

Design, cloning of lentiCRISPR GBM vectors and mTSG sgRNA library, and lentivirus production: Two lentiCRISPR vectors were designed, one for constitutive, and the other for astrocyte-specific genome editing. These vectors contain a cassette that specifically expresses Cre recombinase under the control of an EFS promoter or a GFAP promoter for conditional induction of Cas9 expression in the brain when delivered to LSL-Cas9 mice. Two sgRNA cassettes were built in this vector, one encoding an sgRNA targeting Trp53, with the other being an empty sgRNA cassette (double BsmbI sites for sgRNA cloning) enabling flexible targeting of genes of interest in either individual or pooled manner. These vectors were generated by gBlock gene fragment synthesis (IDT) followed by Gibson assembly (NEB). The mTSG libraries were generated by oligo synthesis, pooled, and cloned into the double BsmbI sites of the lentiCRISPR GBM vectors. The library cloning was done at over 100× coverage to ensure proper representation. Plasmid library representation was readout by barcoded Illumina sequencing as described above, with primers customized to the vectors. The LentiCRISPR GBM plasmid vector (Lenti-vector) and library (Lenti-mTSG) were subjected to high-titre lentivirus production and purification. Briefly, HEK 293FT cells (ThermoFisher) were transiently transfected with transfer (Lenti-vector or Lenti-mTSG), and packaging (psPAX and pMD2.G) plasmids using PEI or Lipofectamine. Each replicate consist of five of 80% confluent HEK 293FT cells in 15-cm tissue culture dishes or T-175 flasks (Corning). Multiple replicates were pooled to enhance production yield. Approximately 48 hours post transfection, virus-containing media was collected, and purified via sucrose gradient ultracentrifugation at >=30,000 rpm for 2-3 h. The supernatant was discarded and the pellet was dried and resuspended with 100 μl sterile PBS at 4° C. overnight. Virus was titered by viral protein p24 ELISA (RnD).

Stereotaxic surgery and virus transduction in the brain: Conditional LSL-Cas9 knock-in mice were bred in a mixed 129/C57BL/6 background. Mixed gender (randomized males and females) 6-14 week old mice were used in the experiment. Animals were maintained and breed in standard individualized cages with maximum of 5 mice per cage, with regular room temperature (65-75° F., or 18-23° C.), 40-60% humidity, and a 12 h:12 h light cycle. Mice were anesthetized by intraperitoneally injection of ketamine (100 mg/kg) and xylazine (10 mg/kg), or by inhalation of isoflurane at approximately 2% for 20-30 minutes. Buprenorphine HCl (0.1 mg/kg), or carprofen (5.0 mg/kg) was administered intraperitoneally as a pre-emptive analgesic. Reflexes were tested before surgical procedures. Once subject mice were in deep anesthesia, they were immobilized in a stereotaxic apparatus (Kopf, or Stoelting) using intra-aural positioning studs and a tooth bar to immobilize the skull. Heat was provided for warmth by a standard heating pad, or a heatlamp. According to the mouse brain stereotaxic coordinates, 1-2 mm holes were drilled on the surface of the skull, and a 33 G Nanofil syringe needle (World Precision Instrument) was used to inject into the ventricle at 0.6-1.0 mm caudal/posterior to Bregma, 0.8-1.5 mm right-side lateral to Bregma, and 2.0-3.0 mm deep from the pial surface for injection (coordinates: A/P −0.6 to −1.0, M/L 0.8 to 1.5, D/V −2.0 to −3.0). For a small fraction of animals, injections were made into the hippocampus (HPF) at the following coordinates (A/P −1.3, M/L 0.6, D/V −1.7). PBS, or 8 uL AAV (between 1×10¹⁰-1×10¹¹ viral genome copies, or Cre copy number equivalent), or 8 uL lentivirus (Between 8×10⁹-8×10¹⁰ viral particles, or p24 equivalent) was injected into the right hemisphere of the brain for each mouse. Injection rates were monitored by an UltraMicroPump3 (World Precision Instruments). After injection, the incision site was closed with 6-0 Ethilon sutures (Ethicon by Johnson & Johnson), or a VetBond tissue glue (3M). Animal were postoperatively hydrated with 1 mL lactated Ringer's solution (subcutaneous) and housed on warmed cages or in a temperature controlled (37° C.) environment until achieving ambulatory recovery. Meloxicam (1-2 mg/kg) was also administered subcutaneously directly after surgery.

MRI: MRI imaging was performed using standard imaging protocols with MRI machines (Varian 7T/310/ASR-whole mouse MRI system, or Bruker 9.4T horizontal small animal systems). Briefly, animals were anesthetized using isoflurane, and setup in the imaging bed with a nosecone providing constant isoflurane. A total of 20-30 views were acquired for each mouse brain using a custom setting: echo time (TE)=20, repetition time (TR)=2000, slicing=0.5 mm. Raw image stacks were processed using Osirix or Slicer tools. Rendering and quantification were performed using Slicer (slicer dot org). For all mice with brain tumors, only 1 tumor was observed per mouse. Tumors were approximate as spheres and their sizes were calculated with the following formula:

Volume(mm³)=0.5*length(mm)*height(mm)*depth(mm)

Survival analysis: Mice that developed brain tumors rapidly deteriorated in their body condition scores. Mice with observed macrocephaly and body condition score <=1 were euthanized and the euthanasia date was recorded as the last survival date. Occasionally mice bearing brain tumors died unexpectedly early, and the date of death was recorded as the last survival date. Cohorts of mice stereotaxically injected with PBS, AAV-vector or AAV-mTSG virus were monitored for their survival. Survival analysis was analyzed using the standard Kaplan-Meier method. Of note, several AAV-vector or PBS injected mice were sacrificed at time points earlier than 299 days (at times when a certain AAV-mTSG mice were found dead or euthanized due to poor body conditions) to provide time-matched histology, but those mice were healthy without brain tumor or other detectable symptoms. Mice euthanized early in healthy states were excluded from calculation of survival percentage.

Mouse brain dissection, fluorescent imaging, and histology: Mice were sacrificed by carbon dioxide asphyxiation or deep anesthesia with isoflurane followed by cervical dislocation. Mouse brains were manually dissected under a fluorescent stereoscope (Zeiss, Olympus or Leica). Brightfield and/or GFP fluorescent images were taken for the dissected brain, and overlaid using ImageJ. Brains were then fixed in 4% formaldehyde or 10% formalin for 48 to 96 hours, embedded in paraffin, sectioned at 6 μm and stained with hematoxylin and eosin (H&E) for pathology. For tumor size quantification, H&E slides were scanned using an Aperio digital slidescanner (Leica). Tumors were manually outlined as region-of-interest (ROI), and subsequently quantified using ImageScope (Leica). Sections were de-waxed, rehydrated and stained using standard immunohistochemistry (IHC) protocols as previously described (Chen et al., 2015, Cell 160, 1246-1260). The following antibodies were used for IHC: rabbit anti-Ki67 (abcam ab16667, 1:500), rabbit anti-GFP (ThermoFisher Scientific A11122, 1:300), rabbit anti-GFAP (Dako, 1:500), and mouse anti-Cas9 (Diagenode, 1:300).

Mouse tissue collection for molecular biology: Mouse brain (targeting organ) and liver (non-targeting organ) were dissected and collected manually. For molecular biology, tissues were flash frozen with liquid nitrogen, ground in 24 Well Polyethylene Vials with metal beads in a GenoGrinder machine (OPS diagnostics). Homogenized tissues were used for DNA/RNA/protein extractions using standard molecular biology protocols.

Genomic DNA extraction from cells and mouse tissues: For genomic DNA (gDNA) extraction, 50-200 mg of frozen ground tissue were resuspended in 6 ml of Lysis Buffer (50 mM Tris, 50 mM EDTA, 1% SDS, pH 8) in a 15 ml conical tube, and 30 μl of 20 mg/ml Proteinase K (Qiagen) was added to the tissue/cell sample and incubated at 55° C. overnight. The next day, 30 μl of 10 mg/ml RNAse A (Qiagen) was added to the lysed sample, which was then inverted 25 times and incubated at 37° C. for 30 minutes. Samples were cooled on ice before addition of 2 ml of pre-chilled 7.5M ammonium acetate (Sigma) to precipitate proteins. The samples were vortexed at high speed for 20 seconds and then centrifuged at ≥4,000×g for 10 minutes. Then, a tight pellet was visible in each tube and the supernatant was carefully decanted into a new 15 ml conical tube. Then 6 ml 100% isopropanol was added to the tube, inverted 50 times and centrifuged at >4,000×g for 10 minutes. Genomic DNA was visible as a small white pellet in each tube. The supernatant was discarded, 6 ml of freshly prepared 70% ethanol was added, the tube was inverted 10 times, and then centrifuged at >4,000×g for 1 minute. The supernatant was discarded by pouring; the tube was briefly spun, and remaining ethanol was removed using a P200 pipette. After air-drying for 10-30 minutes, the DNA changed appearance from a milky white pellet to slightly translucent. Then, 500 μl of ddH₂O was added and the tube was incubated at 65° C. for 1 hour and at room temperature overnight to fully resuspend the DNA. The next day, the gDNA samples were vortexed briefly. The gDNA concentration was measured using a Nanodrop (Thermo Scientific).

Targeted capture sequencing probe design: Targeted capture sequencing probes were designed as follows: the predicted cutting sites (3 bp 5′ of PAM) of the 280 gene-targeting sgRNAs in the mTSG library plus the Trp53-targeting sgRNA in the vector were retrieved from mouse genome (mm10). The 140 bp sequences of the flanking regions of the cutting sites (5′-70 bp and 3′-70 bp) were retrieved using Bedtools (Quinlan & Hall, 2010, Bioinformatics 26, 841-842). The regions were consolidated using NimbleDesign (Roche/NimbleGen), and probe matches were set with the following parameters: Preferred Close Matches=3, where initial selection of probes for a given region only included probes with 3 close matches or less; and Maximum Close Matches=20, if there were insufficient probes available for a given region at the Preferred Close Match number, the threshold was incrementally increased to 20 until adequate coverage was achieved. After consolidation, a number of 178 regions covering 280 sgRNAs, with a total of 33638 bp were covered in the probeset, with Target Bases Covered=32239 (95.8%) and one target sgRNA without coverage due to a lack of qualified candidate probes in the region.

Targeted capture sequencing: The mTSG-Amplicon targeted capture sequencing probes were synthesized using the SeqCap EZ Probe Pool synthesis procedure (Roche). The capture sequencing was performed following standard Illumina-Roche protocols. Genomic DNA samples from mouse organs were subjected to fragmentation, followed by a library preparation step using KAPA Library Preparation Kit (Illumina). The libraries were then amplified using LM-PCR, hybridized to the mTSG-Amplicon probe pool, washed and recovered, and amplified with multiplexing barcodes using LM-PCR. The multiplexed library was then QC'ed using qPCR, and subjected to high-throughput sequencing using the Hiseq-2500 or Hiseq-4000 platforms (Illumina) at Yale Center for Genome Analysis. 277/278 (99.6%) of unique targeted sgRNAs were captured for all samples from this experiment, with the missing one being Arid1a-sg5.

mTSG sgRNA cutting efficiency measurement: The mouse mTSG sgRNA cutting efficiency measurement was performed similar to the screen with the exception of early sampling. Briefly, AAV-mTSG library virus was injected in to LV of LSL-Cas9 mice, but instead of end-point tumor, mice were sacrificed at early time point (3.5 weeks post injection) and examined under fluorescent stereoscope, and GFP+regions from the brain were dissected. Genomic DNA was extracted, and subjected to capture sequencing.

Mouse whole-exome capture sequencing: The mouse whole-exome capture was performed using SeqCap EZ exome kit (Roche). Briefly, capture sequencing was done following standard Illumina-Roche-Illumina protocols. Genomic DNA samples from mouse organs were subjected to fragmentation, followed by a library preparation step using KAPA Library Preparation Kit (Illumina). The libraries were then amplified using LM-PCR, hybridized to the exome probe pool, washed and recovered, and amplified with multiplexing barcodes using LM-PCR. The multiplexed library was then QC'ed using qPCR, and subjected to high-throughput sequencing using the Hiseq-2500 or Hiseq-4000 platforms (Illumina) at Yale Center for Genome Analysis.

Illumina sequencing data processing and variant calling: FASTQ reads were mapped to the mm10 genome using the bwa mem function in BWA v0.7.13 (Li and Durbin, 2009, Bioinformatics 25, 1754-1760). Bam files were merged, sorted, and indexed using bamtools v2.4.0 (Barnett et al., 2011, Bioinformatics 27, 1691-1692) and samtools v1.3 (Li et al., 2009, Bioinformatics 25, 2078-2079). For each sample, indel variants were called using samtools and VarScan v2.3.9 (Koboldt et al., 2012, Genome Res 22, 568-576). Specifically, samtools mpileup (-d 1000000000 -B -q 10) was used and the output was piped to VarScan pileup2indel (--min-coverage 1 --min-reads2 1 --min-var-freq 0.001 --p-value 0.05). To link each indel to the sgRNA that most likely caused the mutation, the center position of each indel was mapped to the closest sgRNA cut site.

Calling significantly mutated sgRNAs and significantly mutated genes: All detected indels were further filtered by requiring that each indel overlap with the ±3 basepairs flanking the closest sgRNA cut site, as Cas9-induced double-strand breaks are expected to occur within a narrow window of the predicted cut site. A series of criteria to identify high-confidence mutations were utilized: 1) As an initial pass to exclude possible germline mutations, any sgRNAs with indels present in more than half of the control samples with greater than 5% variant frequency were removed. This filter specifically removed Rps19_sg5 from further consideration. 2) To determine the significantly cutting sgRNAs in each sample, a false-discovery approach was used based on the PBS and vector control samples. For each sgRNA, the highest % variant read frequency across all control samples was taken; in order for a mutation to be called in an mTSG sample, the % variant read frequency had to exceed the control sample cutoff. However, since the base vector contained a Trp53 sgRNA (Trp53 sg8) whose cut site was only 1 bp away from the target site of Trp53 sg4 (from mTSG library), only PBS samples were considered when calculating the false-discovery cutoff for Trp53 sg4. Nevertheless, in the current study this exception was unnecessary because of the third filter: 3) To identify the dominant clones in each sample, a 5% variant frequency cutoff was further set on top of the false-discovery cutoff. These criteria gave a binary table (i.e. not significantly mutated vs. significantly mutated detailing each sgRNA and whether its target site was significantly mutated in each sample. None of the AAV-vector samples passed the 5% cutoff at the p53 sg 4/8 target site, which is consistent with our observation that no tumors were found in vector-treated animals. To convert significantly cutting sgRNAs into significantly mutated genes, the binary sgRNA scores were collapsed by gene, such that if any of the 5 sgRNAs for a gene were found to be significantly cutting, the entire gene would be called as significantly mutated.

Exome sequencing data analysis: For exome sequencing analysis, a modified set of criteria were imposed on each detected variant: 1) ≥10 supporting reads for the reference allele; 2) ≥10 supporting reads for the variant allele; 3) the variant is within ±6 bp of a Cas9 PAM, NGG (or CCN on the reverse strand); 4) a variant allele frequency <75%, as this was the maximum detected variant frequency out of the mTSG brain samples; 5) the variant was not detected in any sequenced control samples, which were considered as germline variants.

Clustering of variant frequencies to infer clonality of tumors: For each mTSG brain sample, the individual variants that comprised the SMS calls in that sample were extracted, with a cutoff of 5% variant frequency to eliminate low-abundance variants. Because of these cutoffs, 3 sequenced mTSG brain samples were not eligible for variant frequency clustering analysis. To identify clusters of variant frequencies in an unbiased manner, the variant frequency distribution was modeled with a Gaussian kernel density estimate, using the Sheather-Jones method to select the smoothing bandwidth. From the kernel density estimate, the number of local maxima (i.e. “peaks”) within the density function were identified. The number of peaks thus represents the number of variant frequency clusters for an individual sample, which is an approximation for the clonality of the tumors.

Coding frame analysis: For coding frame and exonic/intronic analysis, only indels that were associated with a sgRNA which had been considered significantly mutated in that particular sample were considered. This final set of significant indels was converted to .avinput format and subsequently annotated using ANNOVAR v. 2016 Feb. 1, using default settings (Wang et al., 2010, Nucleic Acids Res 38).

Co-occurrence and correlation analysis: Co-occurrence analysis was performed by first generating a double-mutant count table for each pairwise combination of genes in the mTSG library. Statistical significance of the co-occurrence was assessed by hypergeometric test. For correlation analysis, the % variant frequency tables was collapsed on the gene level (in other words, summing the % variant frequencies for all 5 of the targeting sgRNAs for each gene). Using these summed % variant frequency values, the Pearson correlation was calculated between all gene pairs, across each mTSG sample. Statistical significance of the correlation was determined by converting the correlation coefficient to a t-statistic, and then using the t-distribution to find the associated probability. A similar approach was used to analyze co-occurring mutations in human TCGA GBM data.

Testing driver combinations with sgRNA minipool: Mixtures of five sgRNAs targeting each gene were cloned as sgRNA minipool into the same astrocyte-specific AAV-CRISPR vector. For gene pair targeting, the five-sgRNA single gene minipools from both genes were mixed 1:1. Plasmid mixes were then packaged into AAV½. Briefly, HEK293FT cells were transfected with the minipools plasmids, pAAV1 plasmid, pAAV2 plasmid, helper plasmid pDF6, and PEI Max (Polysciences, Inc. 24765-2) in DMEM (ThermoFisher, 10569-010). 72 hours post transfection, cell culture media was discarded and cells were rinsed and pelleted via low speed centrifugation. Cells were then lysed and the supernatant containing viruses was applied to HiTrap heparin columns (GE Biosciences 17-0406-01) and washed with a series of salt solutions with increasing molarities. During the final stages, the eluates from the heparin columns were concentrated using Amicon ultra-15 centrifugal filter units (Millipore UFC910024). Titering of viral particles was executed by quantitative PCR using custom Cre-targeted Taqman probes (ThermoFisher). After packaging, AAV minipools were stereotaxically injected into the ventricle of LSL-Cas9 mice. Survival and histology analysis followed injection as described above. Several control (uninjected, EYFP and vector) mice were sacrifice as surrogate histology although they were in good body condition and were subsequently found devoid of tumor.

Generation of Nf1 and Rb1 mutant cell lines from primary GBMs induced by AAV-CRISPR minipools: Autochthonous mouse GBMs were induced by stereotaxic injection with the Nf1 or Rb1 AAV minipool (in the AAV9-sgTrp53-sgX-GFAP-Cre vector described above). Tumor-containing brains were visually inspected under a fluorescent dissecting scope, made into single-cell suspension through physical dissociation plus Collagenase/DNase digestion, and cultured in DMEM supplemented with 10% FBS and Pen/Strep. Growing clones were further established as autochthonous mouse GBM cell lines.

Single sgRNA knockout lentiviral production: Lenti-pHKO-U6-sgBsmBI-EF1 a-Puro-P2A-FLuc was generated by subcloning P2A-Fluc expression cassette into a lentiviral CRISPR knockout vector by Gibson assembly. For the cloning of sgRNA targeting individual genes such as Pten, Arid1b, Mll3 (Kmt2c), B2m, and Zc3h13, the corresponding oligos were synthesized, annealed and cloned into BsmBI linearized lentiviral knockout vector. Lentiviruses were produced by transfecting lentiviral knockout plasmids, together with pMD2.G and psPAX2 into 80-90% confluent HEK293FT cells, with viral supernatants collected 48 and 72 h post-transfection, aliquoted and stored in −80° C.

Generation of NF1+geneX and RB1+geneX knockout cell line: The Nf1 and Rb1 knockout tumor cells were infected by single sgRNA knockout lentiviruses at M.O.I<=0.3 to further knockout desired geneX. 24 h post-infection, lentiviral transduced cells were selected by the addition of 4-8 μg/ml puromycin and were split 2-3 days.

Temozolomide (TMZ) treatment, Cell viability assay, and RNAseq: After 7-9 days' culture under puromycin selection, lentiviruses-infected Nf1 and Rb1 knockout tumor cells were plated in triplicates into 96-well plate at a density of 2.5×10³ cells per well, and ˜5×10⁶ cells were collected at the same time and used for cutting efficiency analysis. One day after plating, either TMZ or DMSO was added at a concentration of 10 μM, 100 μM, 500 μM, 1 mM, and 2 mM. After 3 days of drug/vehicle treatment, cell viability was measured using CellTiter Glo (Promega) according to the manufactures' protocol. Briefly, the CellTiter Glo was equilibrated at room temperature for 1 h before use. The media of 96-well plates was aspirated, and then 50 μL fresh DMEM+10% FBS and 50 μL CellTiter Glo was added. The luminescent signals were readout using EnVision plate reader (PerkinElmer). For RNAseq samples preparation, cell lines harboring specific gene knockouts were cultured for 7-9 days under the selection pressure of puromycin, and then plated into 6-well plates at a density of 2×10⁵ cells per well in triplicates. 24 h after plating, 1 mM TMZ or DMSO in fresh DEME+10% FBS was added and cultured for another 48 h. Then, cellular RNA of control or treated cells were extracted by adding 350 μL TRIzol Reagent (Invitrogen) directly into 6-well plates to lyse the cells, followed by gently shaking the plates and incubation for 5-10 min to complete and lysis and homogenization. Then, 70 μL chloroform was added, vigorously mixed, and centrifuged at 16,000 g for 15 min. Transfer the RNA containing aqueous phase to a new tube and further purified using RNeasy mini kit (Qiagen). After eluting RNA from column using Nuclease free water, the concentrations of sample RNA were normalized into 150-300 ng/uL for RNAseq.

T7 Endonuclease I (T7E1) Assays: The genomic DNA of these cells that were collected after 9 days of puromycin selection was extracted by using QuickExtract™ DNA Extraction Solution (Epicentre), mixed well and incubated at 65° C. for 30-60 min. Then, 1-2 μL of genomic DNA from parental or Lenti-sgRNA transduced cells was used as the template to amplify gene of interest using surveyor primers with thermocycling conditions as 98° C. for 1 min, 35 cycles of (98° C. for 1 s, 60° C. for 5 s, 72° C. for 10 s), and 72° C. for 1 min. The PCR products were gel-purified using QIAquick Gel Extraction Kit from 2% E-gel EX and quantified, followed by PCR products denaturing at 95° C. for 5 min, and annealing by using following conditions: ramp from 95 to 85° C. at a rate of −2° C. per seconds; from 85° C. to 25° C. at a rate of −0.1° C. per second, and 4° C. hold. 1 μL of T7 endonuclease I was added into annealed oligo, and incubated at 37° C. for 60 min to digest the mismatched sites. The digested PCR products were loaded into 2% E-gel EX, and the amount of DNA fragments were quantified. The cutting efficiency was calculated to estimate gene editing using the following formula: Indels (%)=100× (1−(1-fraction cleaved)½).

Transcriptome profiling of different driver combinations in the presence and absence of chemotherapy: Mixtures of five sgRNAs targeting each gene were cloned as sgRNA minipool into the same astrocyte-specific AAV-CRISPR vector. After packaging, AAV minipools were stereotaxically injected into the lateral ventricle of LSL-Cas9 mice. Cell lines were derived from mouse GBMs by single-cell isolation, plating and culture in DMEM media. Additional driver mutations were introduced by lentiCRISPR where applicable. GBM cells with different drivers were treated with DMSO or TMZ for 48 h, and harvested for mRNA-seq for transcriptome profiling. Briefly, total RNA was extracted from cancer cells derived from AAV-CRISPR minipools induced GBM treated with DMSO or TMZ, using commercially available kits (Qiagen/Thermofisher). PolyA-mRNA library was constructed using Illumina TruSeq mRNA library prep kit, and sequenced on Illumina Hiseq 2500 and/or Hiseq 4000 platform.

RNA-seq differential expression analysis: Strand-specific single-end RNA-seq read files were analyzed to obtain transcript level counts using Kallisto, with the settings --rf-stranded -b 100. The counts were subsequently passed to the tximport R package to collapse to gene level counts. Pairwise differential expression analysis between groups was then performed using edgeR with default settings.

Pathway enrichment analysis of differentially expressed transcripts: Using an adjusted p-value cutoff of 0.05, and a log fold change threshold of ±1, we determined the set of genes that were significantly upregulated or downregulated. We then used the resultant gene sets for DAVID functional annotation analysis. We considered a GO category as statistically significant if the Benjamini-Hochberg adjusted p-value was less than 0.05.

GBM comparative cancer genomics analysis using TCGA datasets: Somatic mutation calls, copy number variation calls, RNA-seq expression z-scores, and clinical data containing patient survival information were obtained through cBioPortal for GBM on Nov. 15, 2016. Pearson correlation coefficients were calculated comparing mouse and human mutation frequencies; statistical significance was calculated by converting the correlation coefficient to a t-statistic, and then using the t-distribution to calculate significance.

GBM comparative cancer genomics analysis using Yale Glioma datasets: Somatic mutation calls and copy number variation calls and partial clinical data containing diagnostic information were obtained from Yale Glioma tissue bank and data bank. All patient samples were de-identified. Total event for each patient was calculated as the sum of mutation events and copy number variant events. Pearson correlation coefficients were calculated comparing mouse and human mutation frequencies; statistical significance was calculated by converting the correlation coefficient to a t-statistic, and then using the t-distribution to calculate significance.

Histology analysis of clinical GBM samples from Yale Glioma tissue bank: Histology sections were obtained from the Yale Glioma tissue bank. All patient samples were de-identified. The mutations associated with specific samples were obtained from the Yale Glioma data bank. Slides stained with H&E or anti-GFAP were subsequently scanned using a slidescanner (Leica) and subjected to pathological analysis.

Statistical tests: In addition to the statistical tests detailed above, a two-tailed Welch's t-test was used for comparisons in which group variances were unequal. If the variances were found to be sufficiently equal and the data was normally distributed, a standard two-tailed t-test was used. For evaluating differences in the incidence of tumors in different groups, Fischer's exact test was used.

Data accession: Genomic sequencing (targeted capture, exome) and RNA-seq data have all been deposited in NCBI SRA (project PRJNA393202): targeted capture sequencing (SUB2842710), RNA-seq (SUB2843781), and exome sequencing (SUB2843864). CRISPR reagents (AAV-CRISPR and lentiCRISPR backbone plasmids and mTSG libraries) are available to the academic community and have been submitted through Addgene.

The results of the experiments are now described.

### Example 1: Sterotaxic Injection of an AAV-CRISPR Library Drives Robust Gliomagenesis

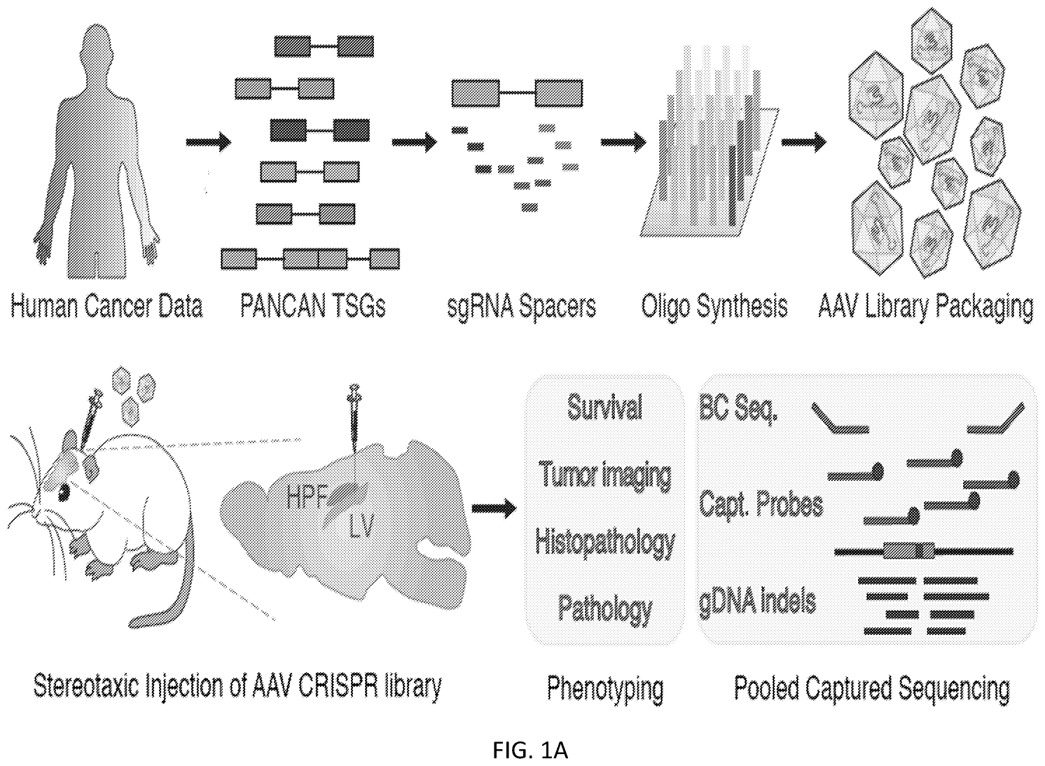

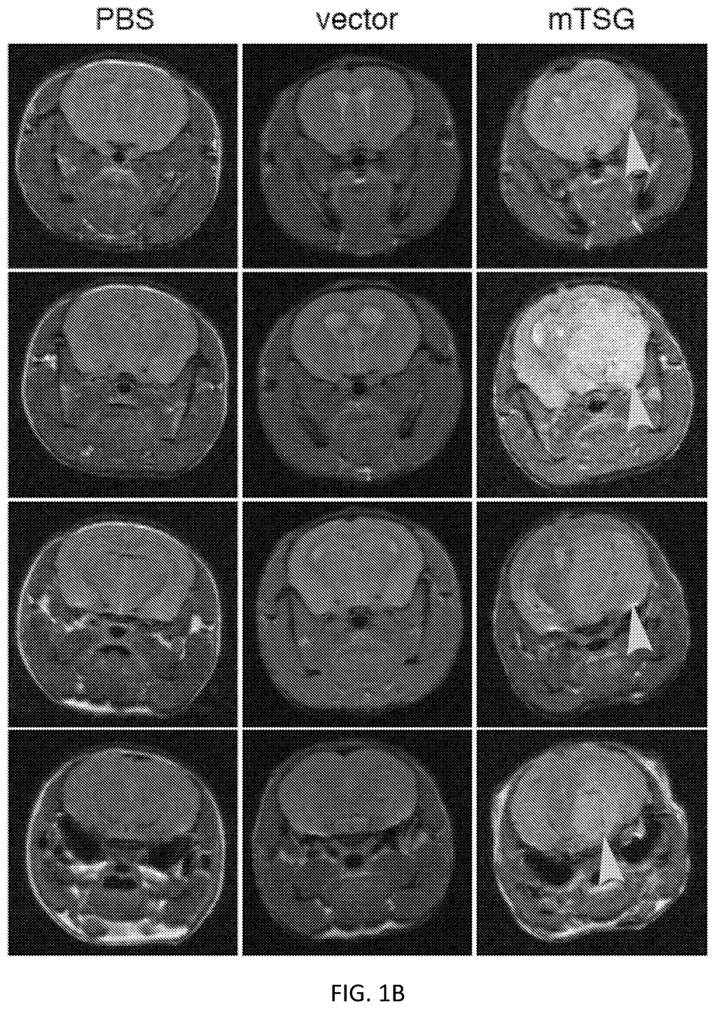

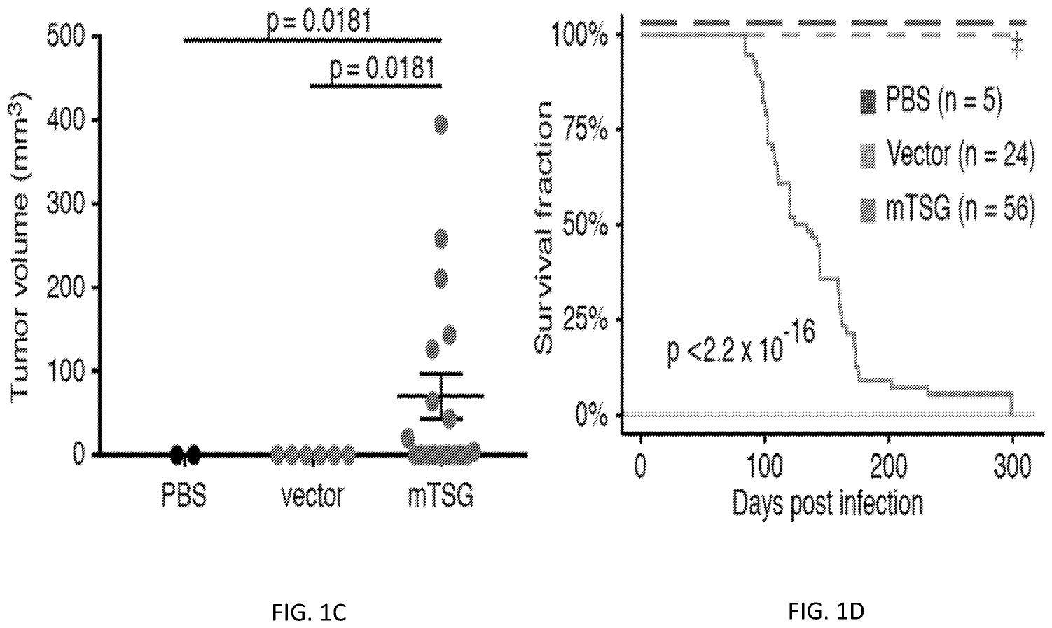

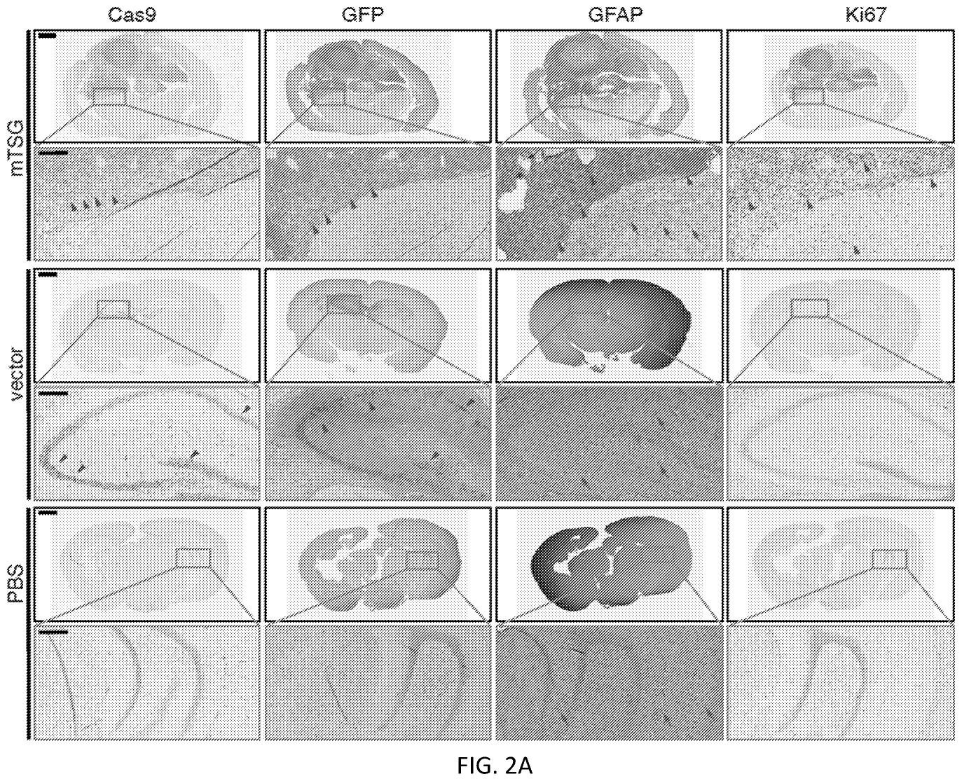

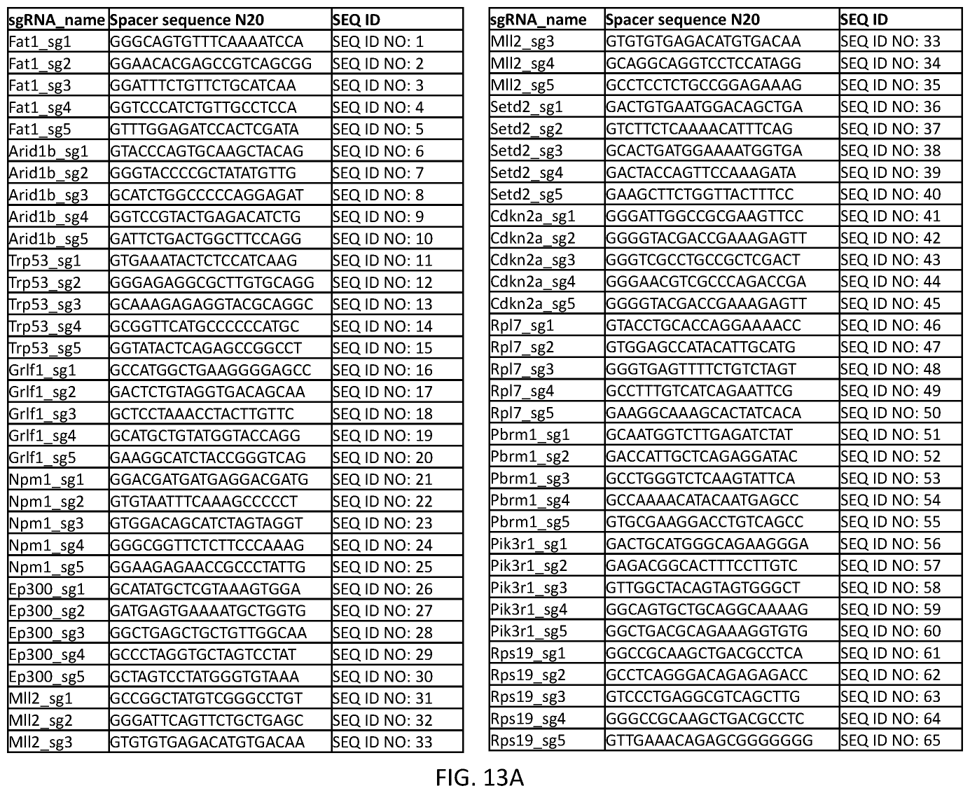

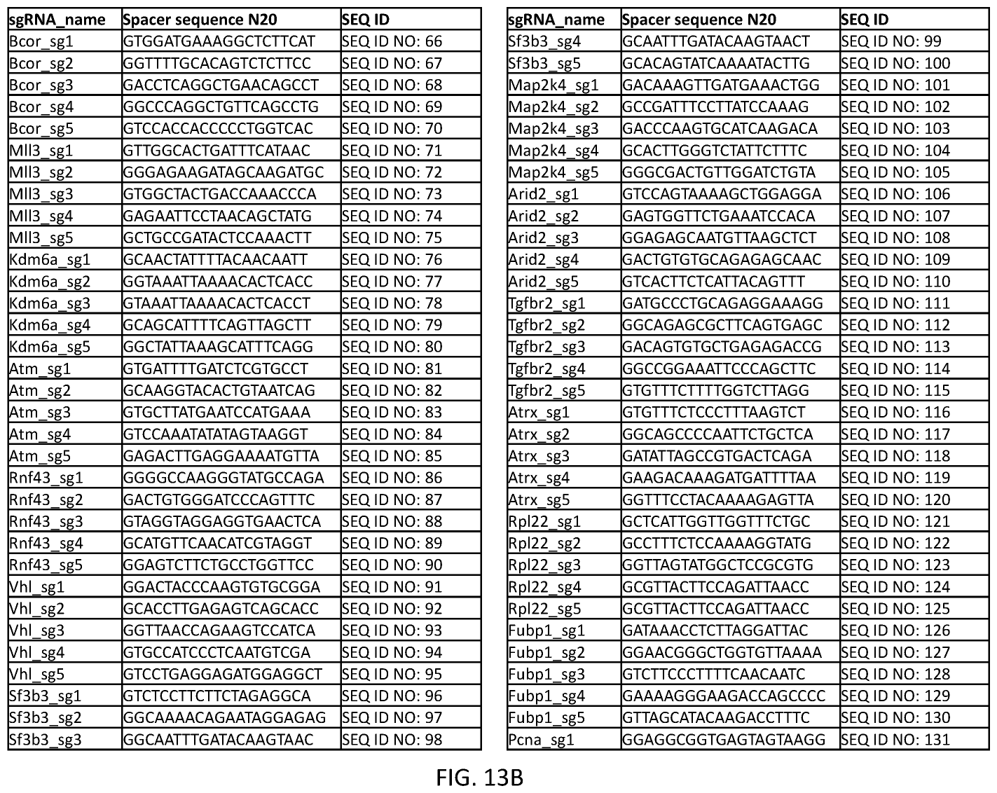

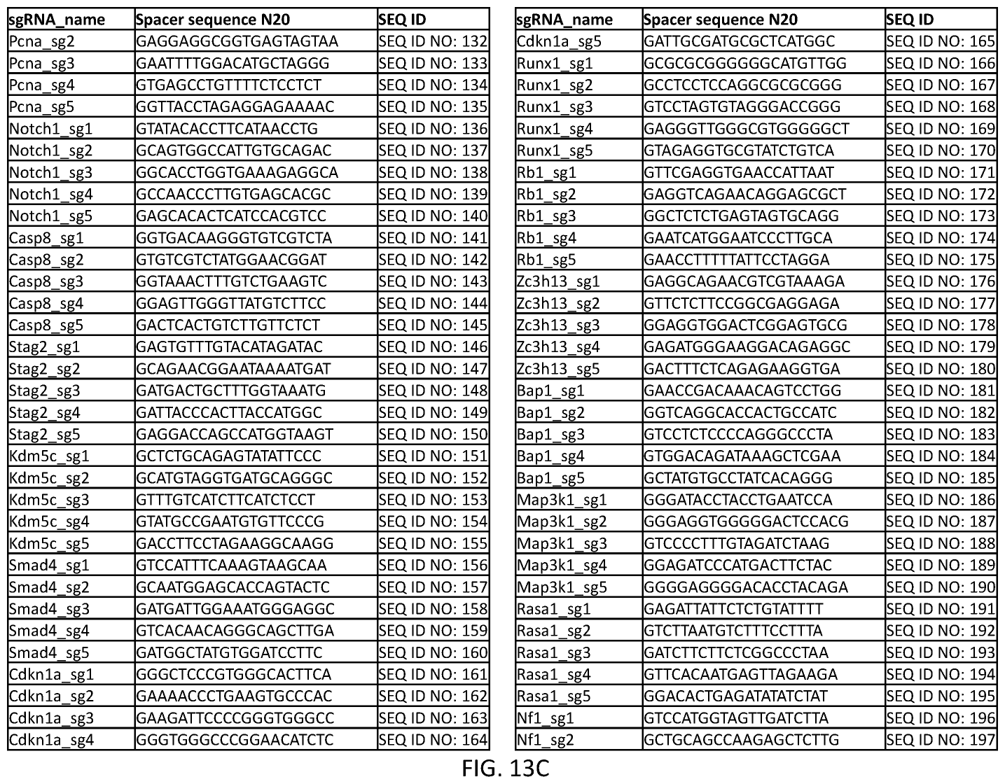

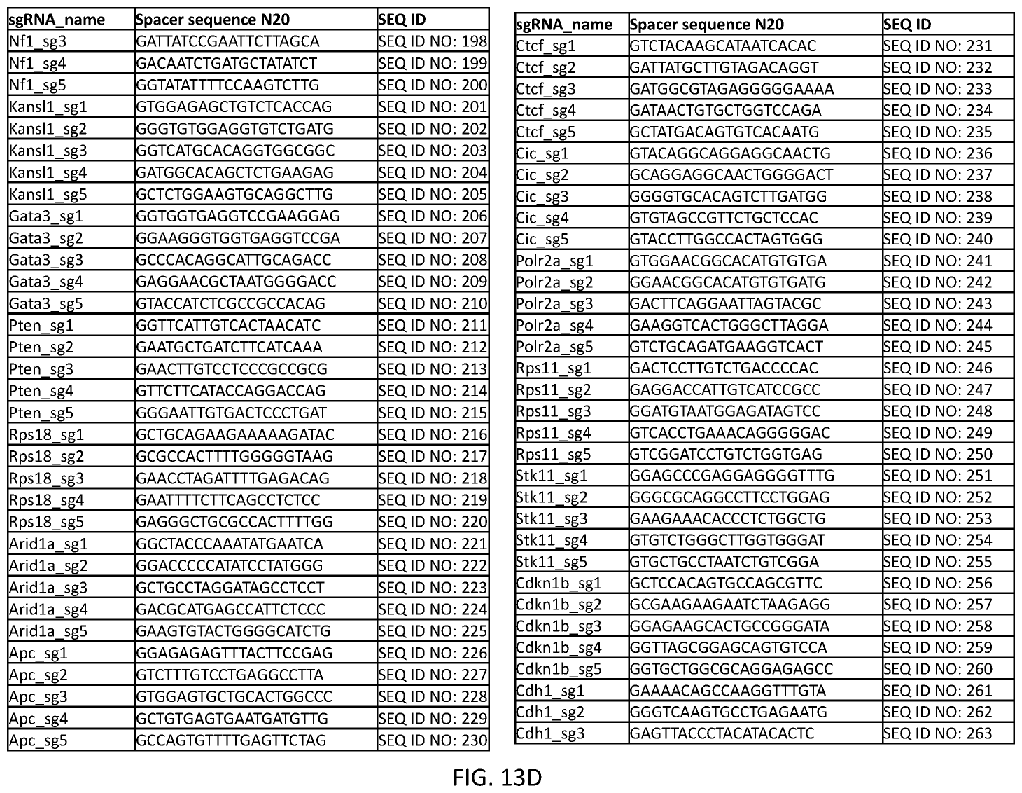

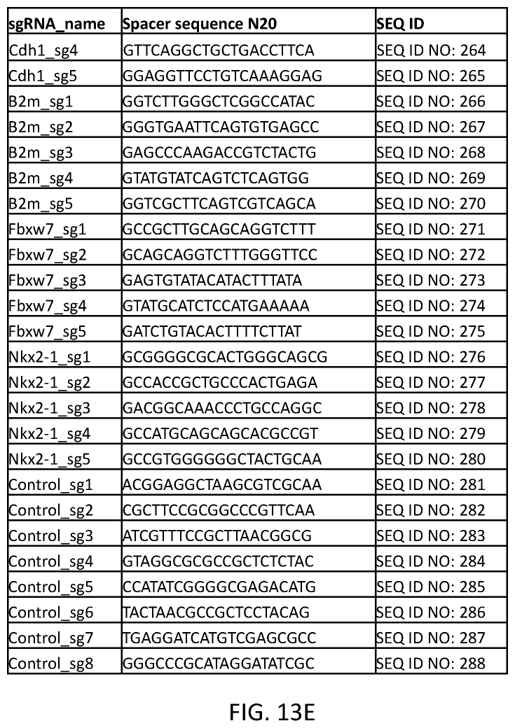

To directly test the function of putative SMGs in the mouse brain, a direct in vivo autochthonous screening strategy was developed, which necessitates pooled mutagenesis of normal cells directly in the native organ and subsequent deconvolution of mutant phenotypes. Based on pan-cancer analysis of mutations in The Cancer Genome Atlas (TCGA) (Davoli et al., 2013, Cell 155, 948-962; Kandoth et al., 2013, Nature 502, 333-339; Lawrence et al., 2014, Nature 505, 495-501; Lawrence et al., 2013, Nature 499, 214-218), a set of the 50 most significantly mutated tumor suppressor genes (TSG) across all cancer types was chosen, and their mouse homologs retrieved (49 genes, one without homolog) (mTSG). An sgRNA library (mTSG library) was designed against these 49 genes together with 7 genes with essential molecular functions as internal controls, with 5 sgRNAs per gene, plus 8 non-targeting controls (NTCs), totaling 288 sgRNAs (FIG. 1A; FIGS. 13A-13E).

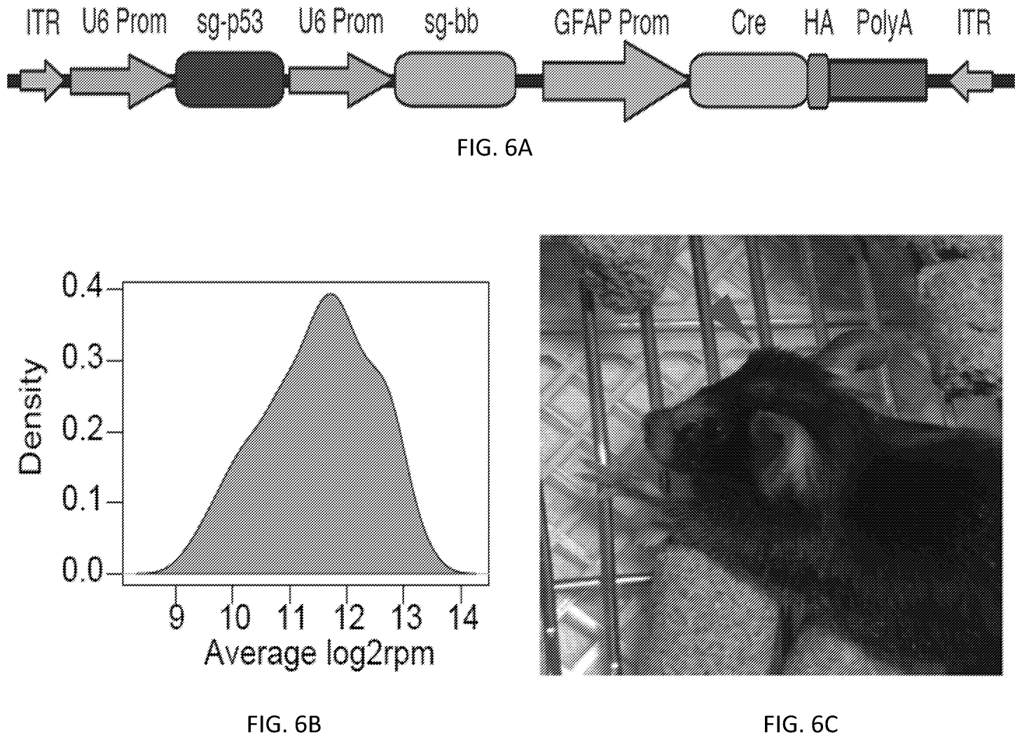

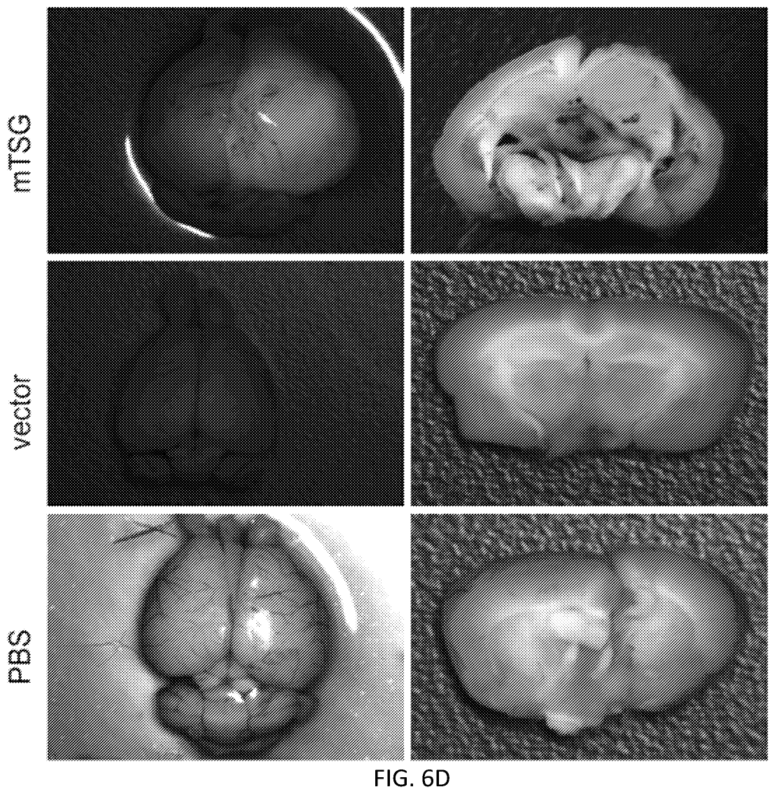

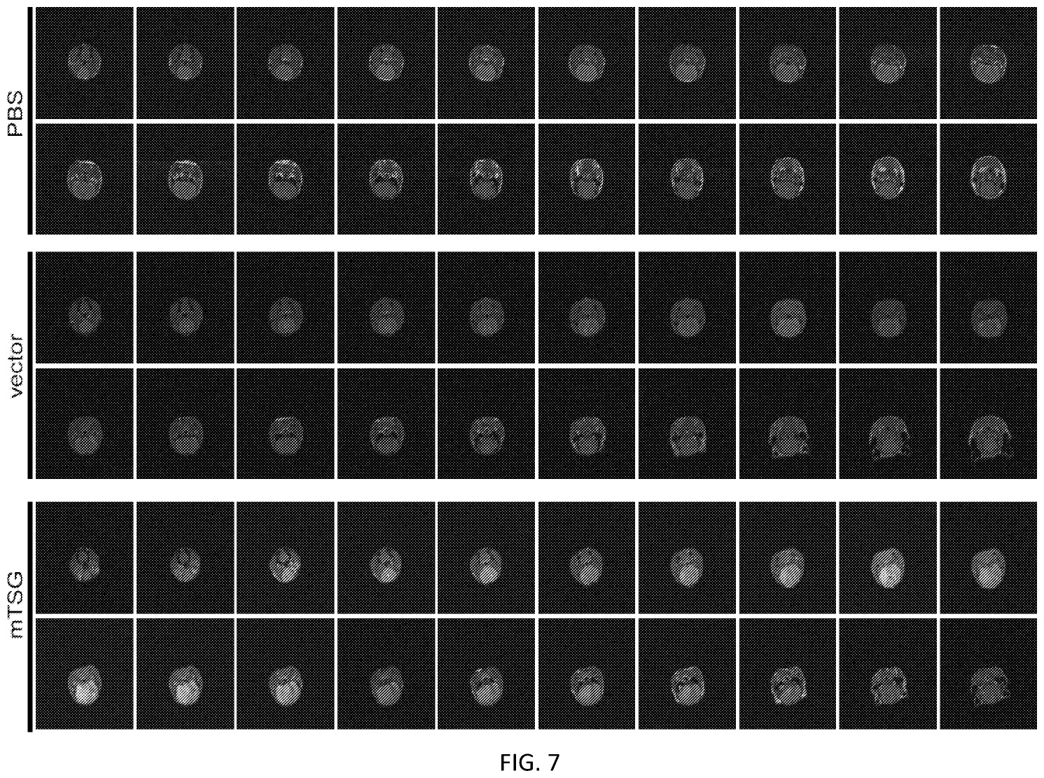

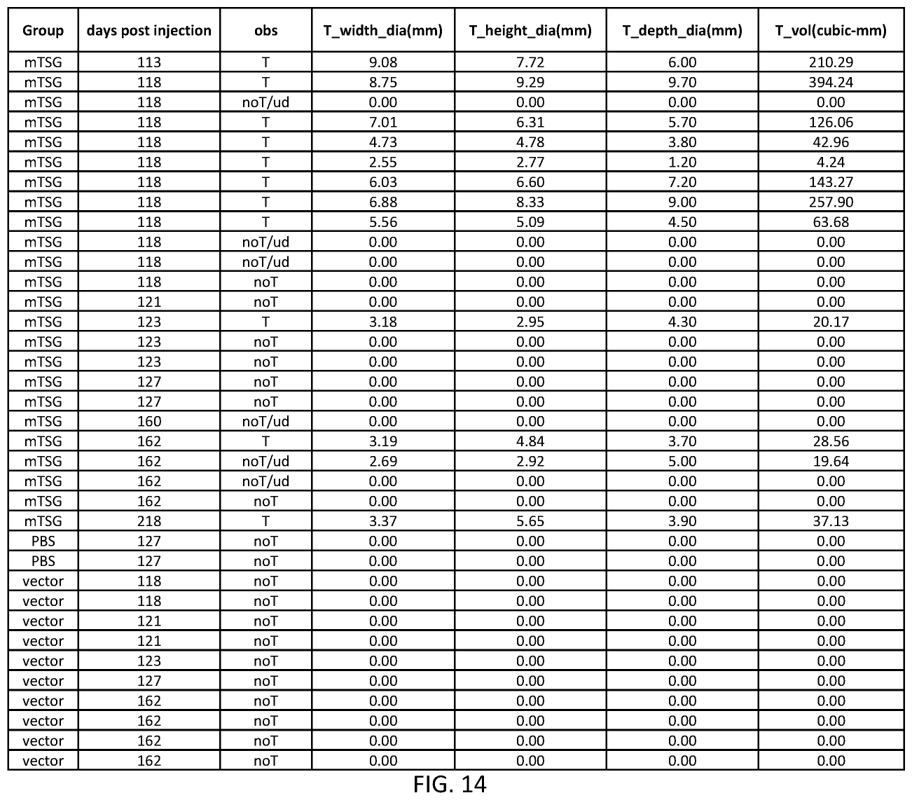

Because GBM is a disease originating from astrocytes, an AAV-CRISPR vector encoding a Glial fibrillary acidic protein (GFAP) promoter driving Cre recombinase for conditional expression of Cas9-GFP in astrocytes was generated. Conditional expression of Cas9 and GFP in astrocytes is achieved when injected into a conditional Rosa26-LSL-Cas9-GFP mouse (LSL-Cas9 mouse) (FIG. 6A). The vector also contains an sgRNA targeting Trp53, with the initial intent to generate co-mutational Trp53 knockouts that might exhibit genome instability and thus be sensitized to tumorigenesis. Local viral delivery into the brain restricts the number of transducible cells, and cancer genomes generally consist of dozens to hundreds of SMGs. With these considerations in mind, an sgRNA library (mTSG library) was designed that targeted the mouse homologs of top-ranked pan-cancer SMGs, plus 7 genes with essential molecular functions that were initially considered as internal controls (FIG. 1A). All designed sgRNAs were synthesized as a pooled library, cloned into the AAV-CRISPR vector at greater than 100× coverage, and deep-sequenced to ensure all sgRNAs were fully covered and represented with a tight lognormal distribution (FIG. 1A, FIG. 6B). High-titer AAVs (>1*10¹² viral particles per mL) were generated from the vector containing mTSG library (termed AAV-mTSG), as well as the empty vector (termed AAV-vector) (FIG. 1A). AAV-mTSG, AAV-vector, or PBS was stereotaxically injected into the lateral ventricle (LV, n=40 mice) (x=0.8˜1.5, y=−0.6˜−1.0, z=−2.0˜−3.0) or hippocampus (HPF, n=16 mice) (x=0.6, y=−1.3, z=−1.7) in the brains of LSL-Cas9 mice. At four-months post-injection, magnetic resonance imaging (MRI) was performed to scan the brains of these mice. Half ( 9/18=50%) of AAV-mTSG library transduced animals developed brain tumors, whereas none of AAV-vector or PBS injected animals had detectable tumors by MRI (FIG. 1B; FIG. 7; FIG. 14). Quantification of tumor volumes showed that AAV-mTSG transduced mice had average tumor volumes of 70.2 mm³ (including animals without tumors), or 140.3 mm³ (excluding animals without tumor) (two-tailed Welch's t-test, t₁₇=2.62, p=0.018, mTSG vs. vector or PBS) (FIG. 1C; FIG. 14). These data indicated that the AAV-mTSG viral library had driven tumorigenesis in the brain of LSL-Cas9 mice.

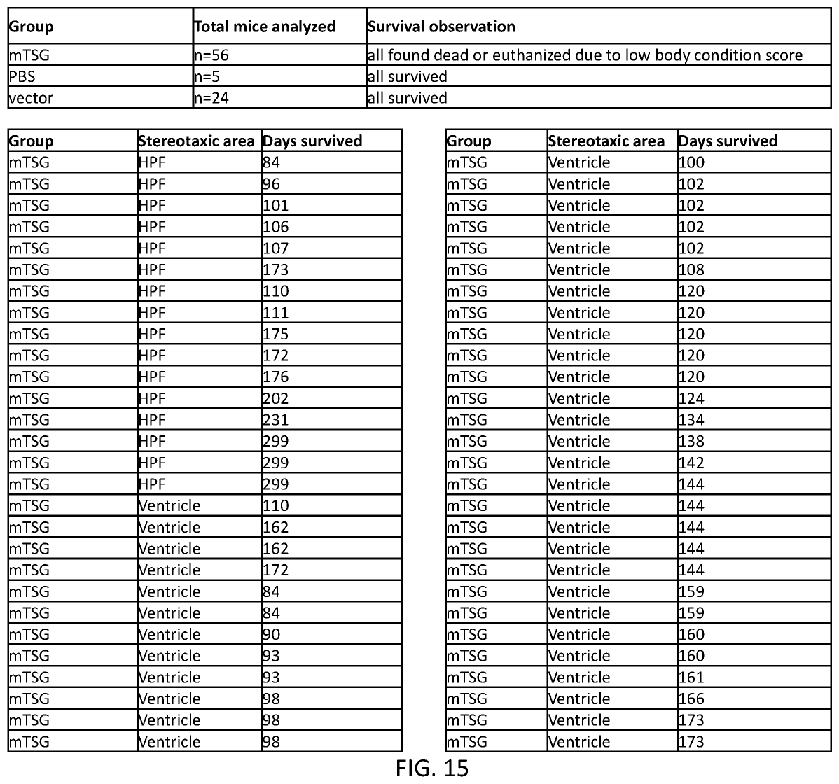

The overall survival of a cohort of LSL-Cas9 mice injected with AAV-mTSG, AAV-vector or PBS was analyzed (FIG. 15). In this screen, injection location did not affect the rate of tumor development as reflected by overall survival (two-sided Mann-Whitney U test of HPF vs LV, p=0.054) (FIG. 15), and thus were considered as one group (AAV-mTSG). For the AAV-mTSG transduced group, the first three animals died 84 days post injection (dpi), 90% of animals did not survive 176 dpi, and all 56 AAV-mTSG transduced animals died (or were too sick to survive and were euthanized) within 299 days (FIG. 15; FIG. 1D). The median survival time of the AAV-mTSG group was 129 days (95% confidence interval (CI)=111 to 159 days), and 90% of animals did not survive 176 dpi (FIG. 1D), consistent with the presence of tumors in half of the mice at 4 months by MRI. In sharp contrast, all 24 AAV-vector and all 5 PBS injected animals survived the duration of the study and maintained good body condition (BCS=S) (log-rank (LR) test, p<2.2e-16, mTSG vs. vector or PBS; LR test, p=1, vector vs PBS) (FIG. 1D). For the vast majority (96.4%, or 54/56) of AAV-mTSG injected mice, macrocephaly was observed at the end point (FIG. 6B), suggesting that they developed brain tumors. On the contrary, macrocephaly was observed in none of the AAV-vector ( 0/24) or PBS ( 0/5) injected mice during the whole study (Two-tailed Chi-square test, p<1e-5, mTSG vs. vector or PBS; Chi-square test, p=1, vector vs PBS, two-tailed Fischer's exact test, p<1*10⁵′ mTSG vs. vector or PBS; p=1, vector vs. PBS). These data indicated that the AAV-mTSG viral library induced brain tumors of LSL-Cas9 mice at high penetrance and led to lethality.

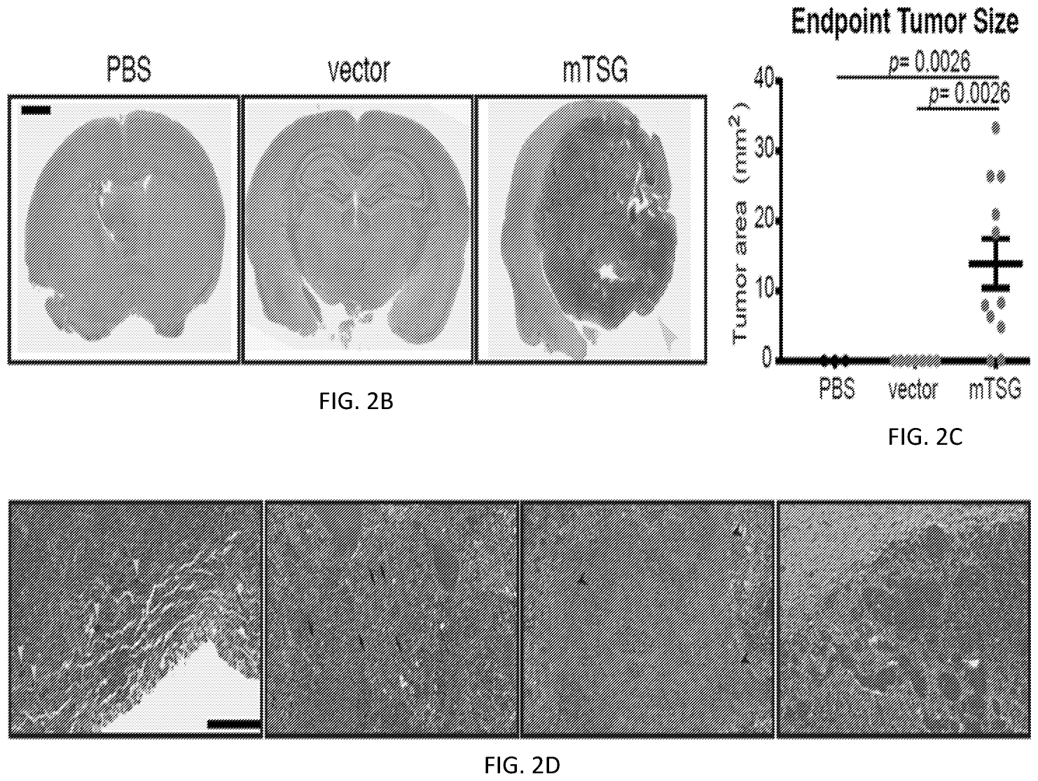

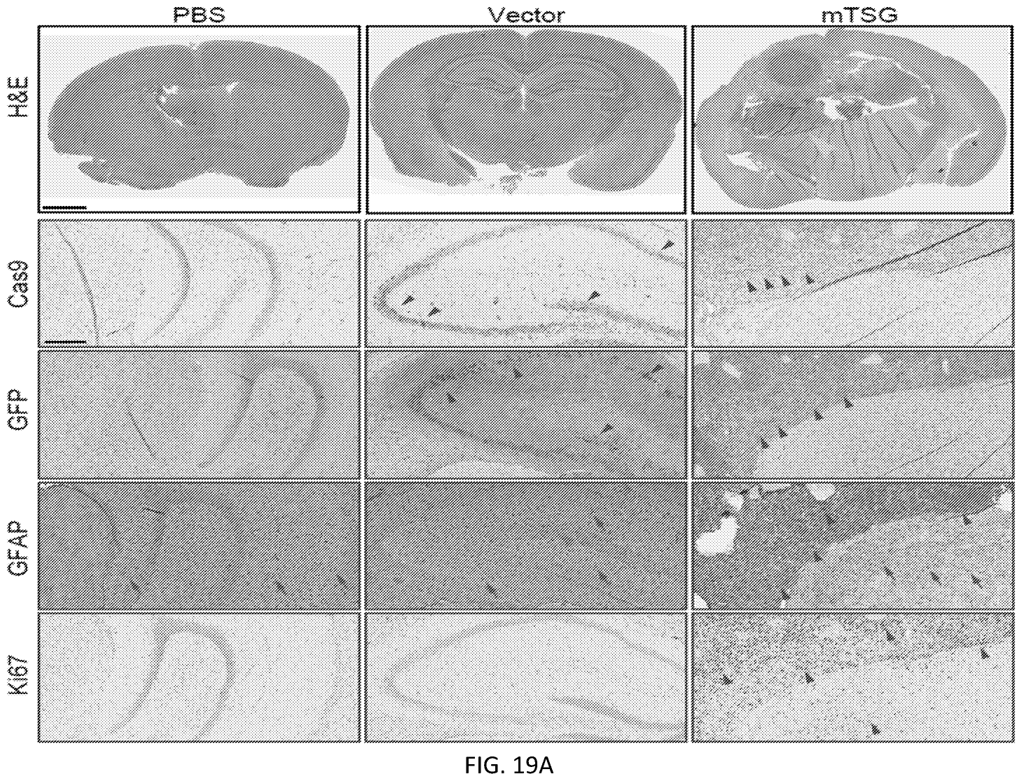

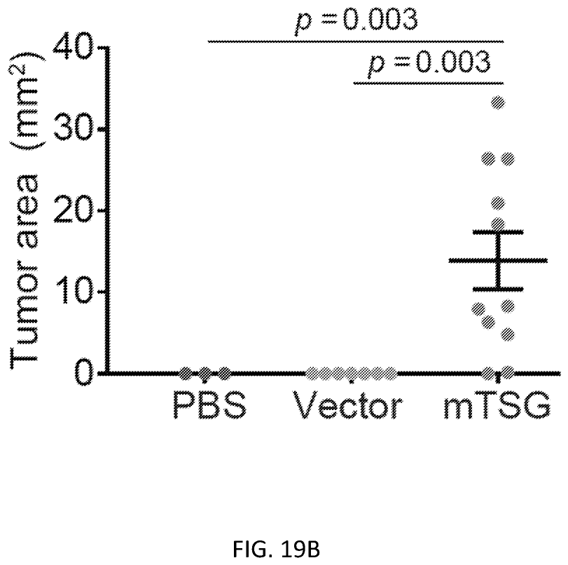

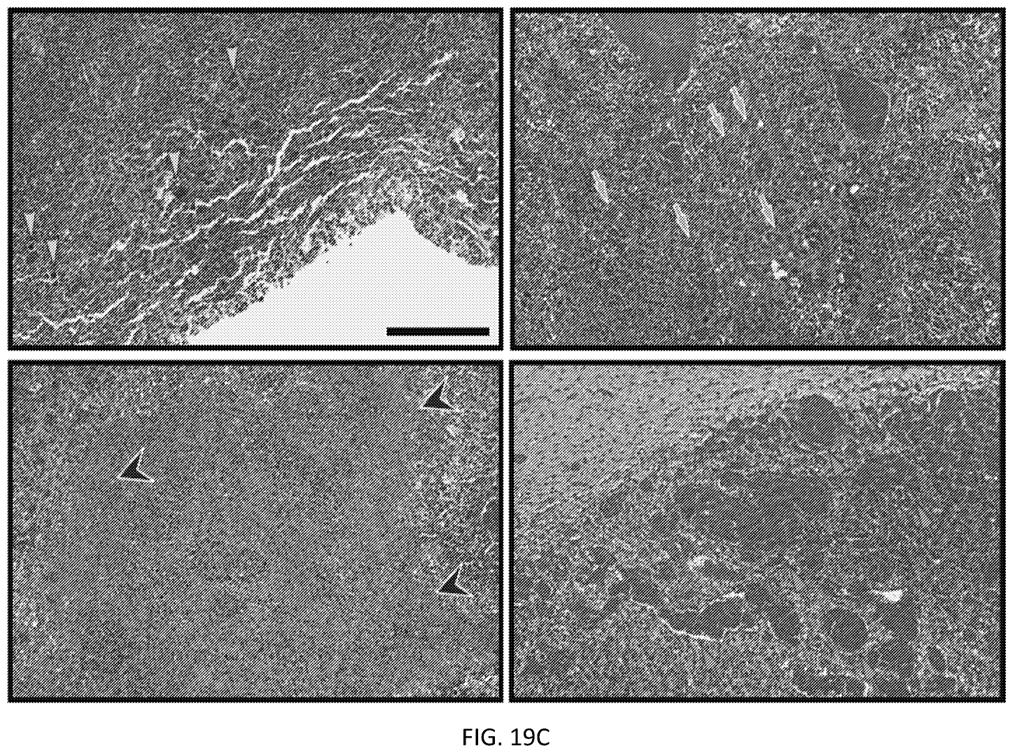

For several AAV-mTSG mice at the time of euthanasia, together with time-matched cohorts of AAV-vector or PBS injected, or uninjected mice, the brains of these mice were dissected and examined under a fluorescent stereoscope. In accordance with the prior MRI analysis, AAV-mTSG mice had massive GFP-positive tumors that deformed the brains (100% or 6/6) (FIG. 6D; FIG. 19A). AAV-vector mice had GFP-positive regions in the brain with fully normal morphology, suggesting these were AAV-transduced cells expressing Cas9-GFP induced by Cre expression that had not become tumors (n=2) (FIG. 6D; FIG. 19A). PBS injected or uninjected mice had no detectable GFP expression even at long exposure (n=3) (FIG. 6D; FIG. 19A). Immunohistochemistry (IHC) analysis showed that AAV-mTSG induced tumors stained positive for Cas9 and GFP, consistent with them having arisen from cells with activation of Cas9-GFP expression (FIG. 2A; FIG. 19A); positive for GFAP, consistent with their astrocytic origin (FIG. 2A; FIG. 19A); and for Ki67, a proliferation marker (FIG. 2A; FIG. 19A). AAV-vector transduced brains stained positive for Cas9 and GFP in a subset of cells at the injection site (FIG. 2A; FIG. 19A), but these cells were not proliferative (Ki67 negative) maintained normal morphology (did not have tumor-like pathological features) (FIG. 2A; FIG. 19A). PBS injected mice stained negative for Cas9 and Ki67 (FIG. 2A; FIG. 19A).

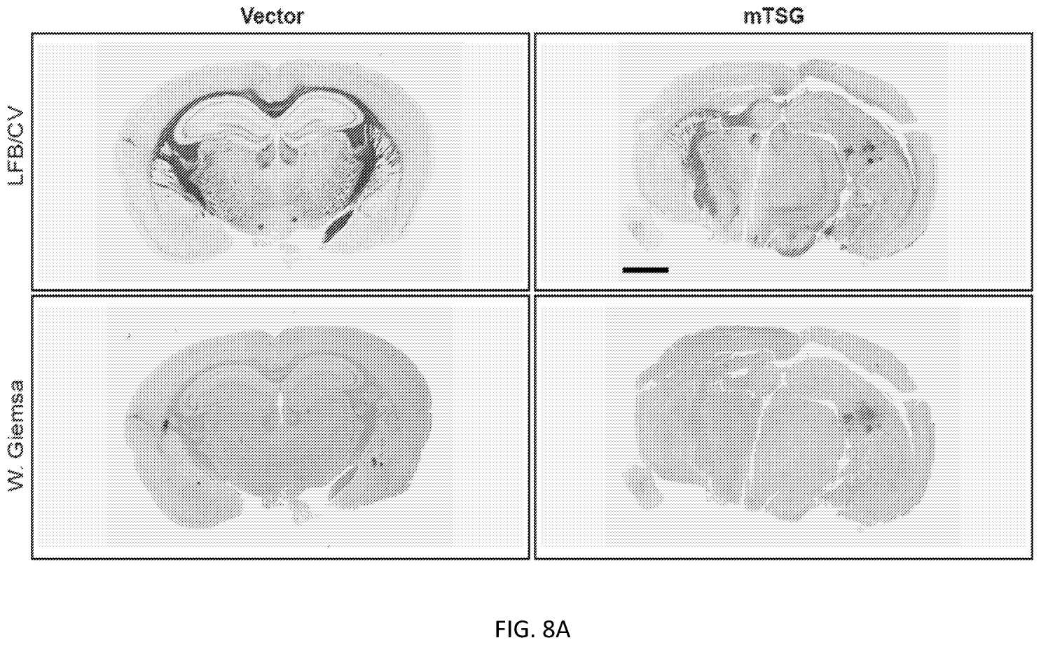

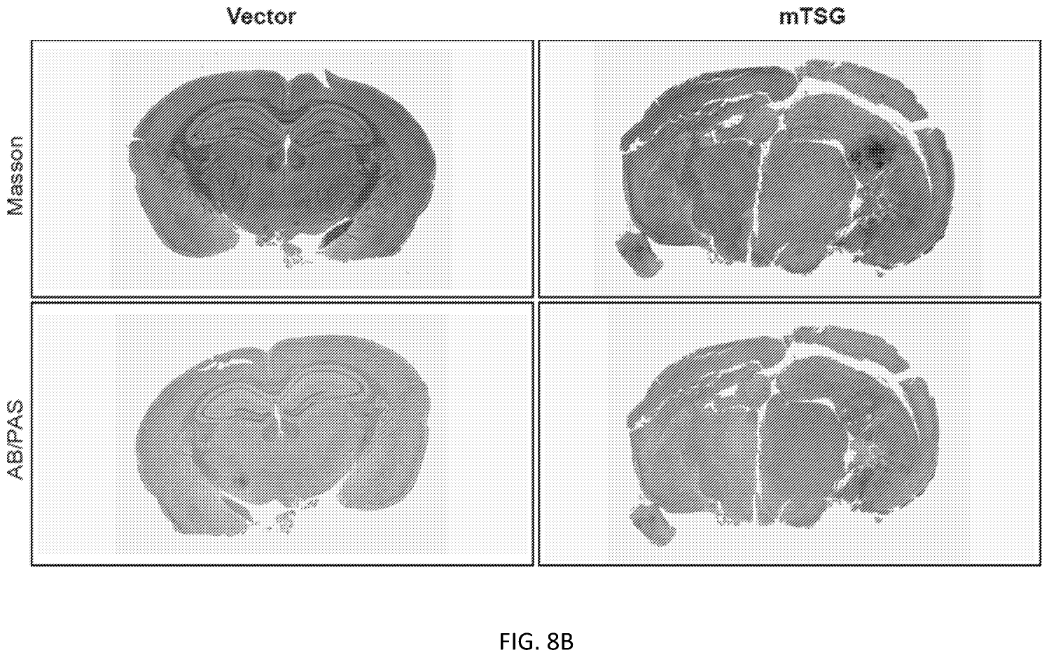

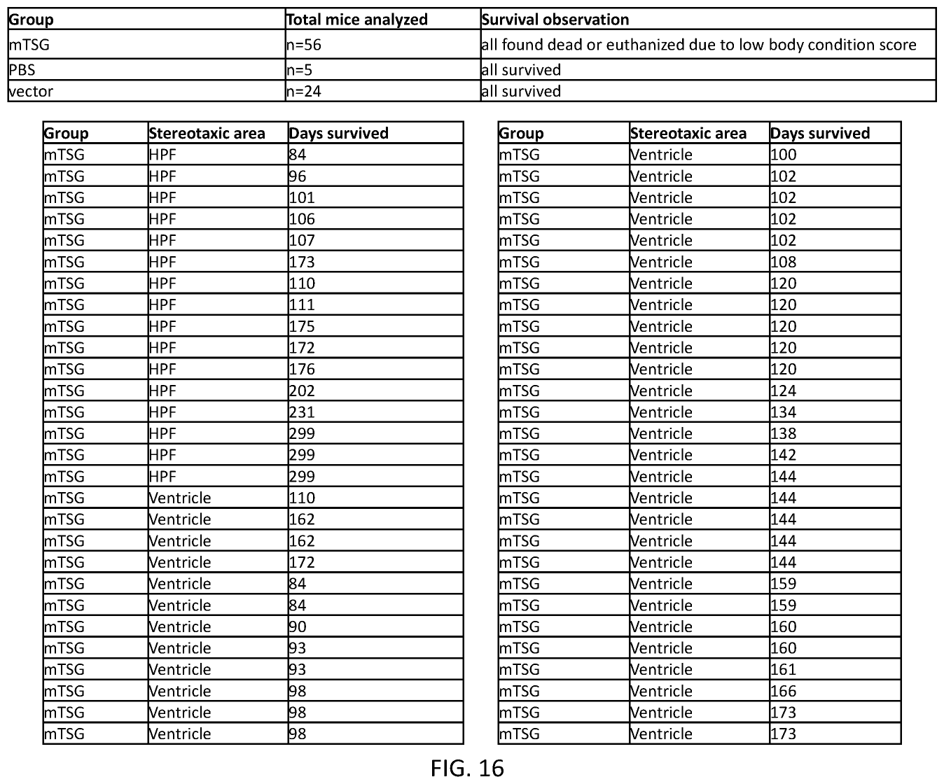

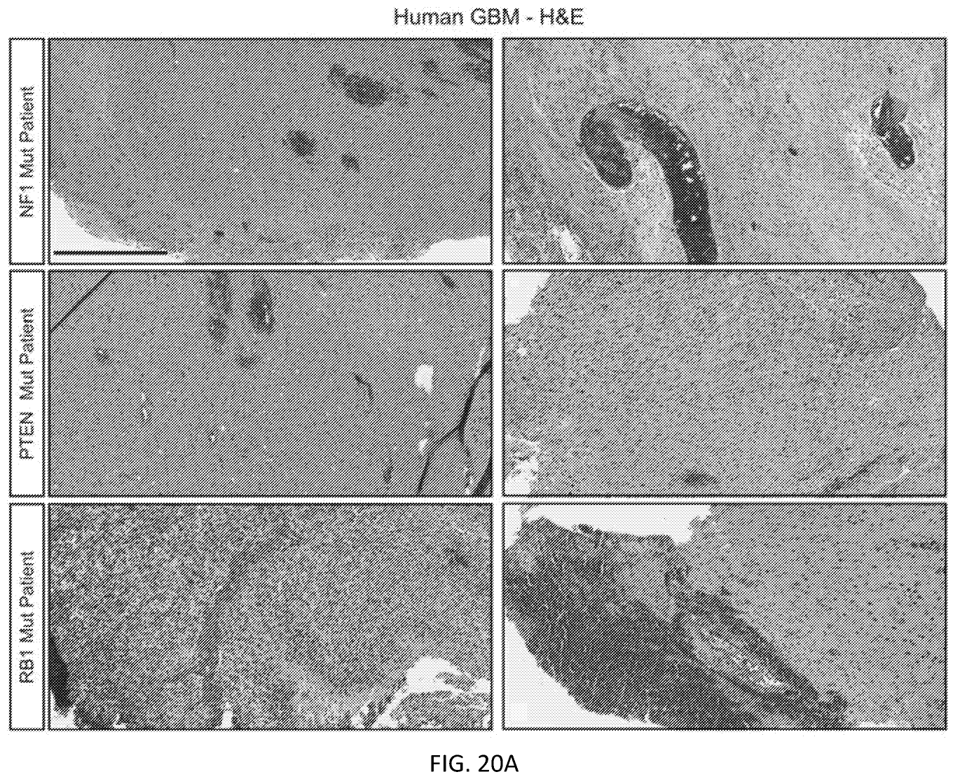

Endpoint histopathology showed that the vast majority of AAV-mTSG mice developed brain tumors ( 10/11=91%), whereas none of AAV-vector ( 0/7=0%) or PBS ( 0/3=0%) mice had detectable tumors, as shown by endpoint histopathology analysis (two-tailed Fischer's exact test: p=0.0003, mTSG vs. vector; p=0.011, mTSG vs. PBS (FIG. 2B; FIGS. 19A-19B; FIG. 16). The mean endpoint tumor size as measured by area in the brain sections for the AAV-mTSG group was 13.9 mm², as compared to 0 mm² in the two control groups (two-tailed Welch's t-test, t₁₀=3.97, p=0.003, mTSG vs. vector or PBS) (FIG. 2C; FIG. 19B). The brain tumors in AAV-mTSG mice showed pathological features of dense cellular structure with proliferative spindles, nuclear aneuploidy and pleiomorphism, giant cells, regions of necrosis, angiogenesis and hemorrhage (FIG. 2D; FIG. 19C), all of which are hallmark features of human GBM. Clinical features such as deformation of brain, invasion, loss of neuronal bundles, necrosis and hemorrhage were further corroborated by special staining methods such as Luxol fast blue Cresyl violet (LFB/CV), Wight Giemsa, Masson and Alcian blue Periodic acid—Schiff (AB/PAS) (FIGS. 8A-8B). A panel of human GBM clinical samples from Yale Glioma tissue bank was investigated, and the observation of these pathological features was confirmed (FIGS. 20A-20B). These data suggest that the AAV-mTSG library-induced autochthonous brain tumors recapitulate various histological and pathological features of human GBM.

### Example 2: Targeted Capture Sequencing Reveals Diverse Mutational Profiles Across Tumors

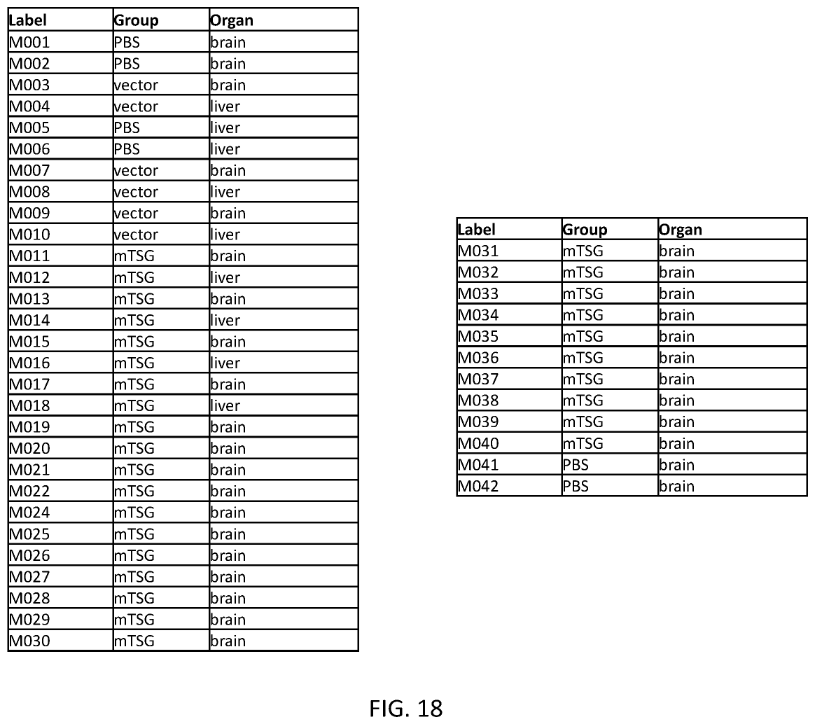

Because AAVs usually do not integrate into the genome, direct sequencing of the targeted regions was needed to determine which mutations were in each tumor. To globally map the molecular landscape of these brain tumors, a customized probe set was designed (mTSG-Amplicon probes) covering the targeted regions of all library sgRNAs (FIGS. 17A-17F). These probes were used to perform targeted captured sequencing for whole brain samples and liver samples (as a control organ not being directly transduced) in a cohort of AAV-mTSG, AAV-vector and PBS injected mice (n=25, 3, and 4 brain samples, respectively) (FIG. 18). As a result, 277/278 (99.6%) of unique sgRNA target regions were captured for all samples from this experiment, the exception being Arid1a-sg5 (due to unavailability of qualified regions in capture probe design). Across all 41 brain and liver samples, the average mean coverage across all probes was 19,405±180, providing sufficient sequencing depth for detecting mutant variants at frequencies as low as 0.01%.

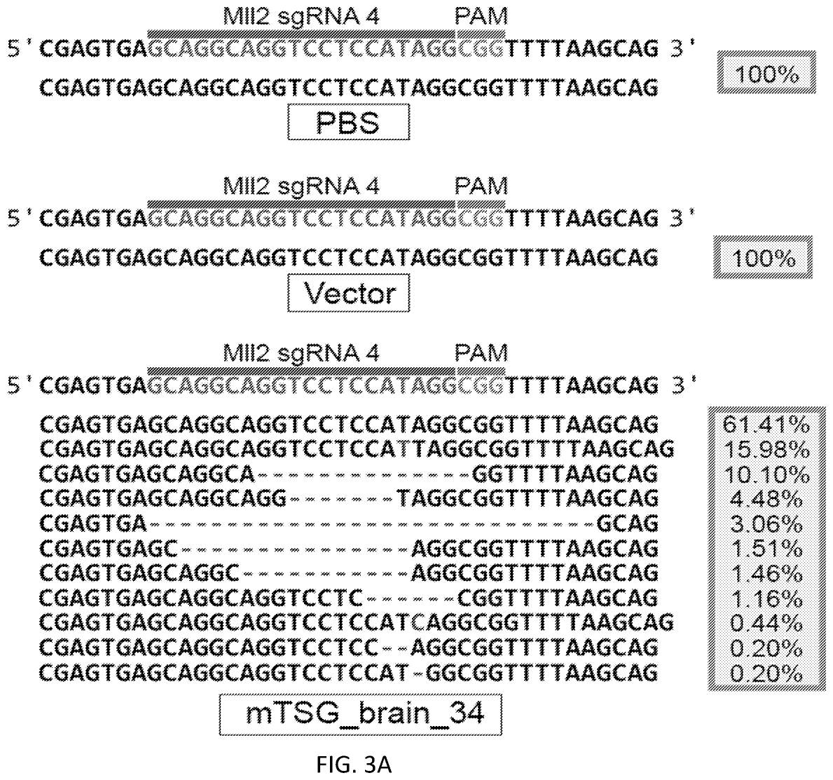

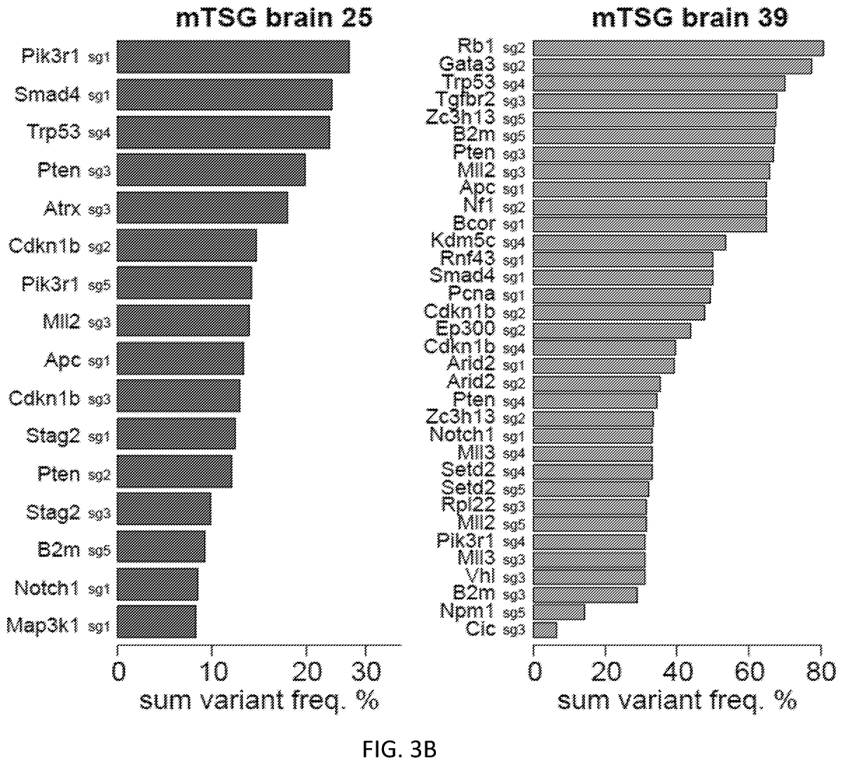

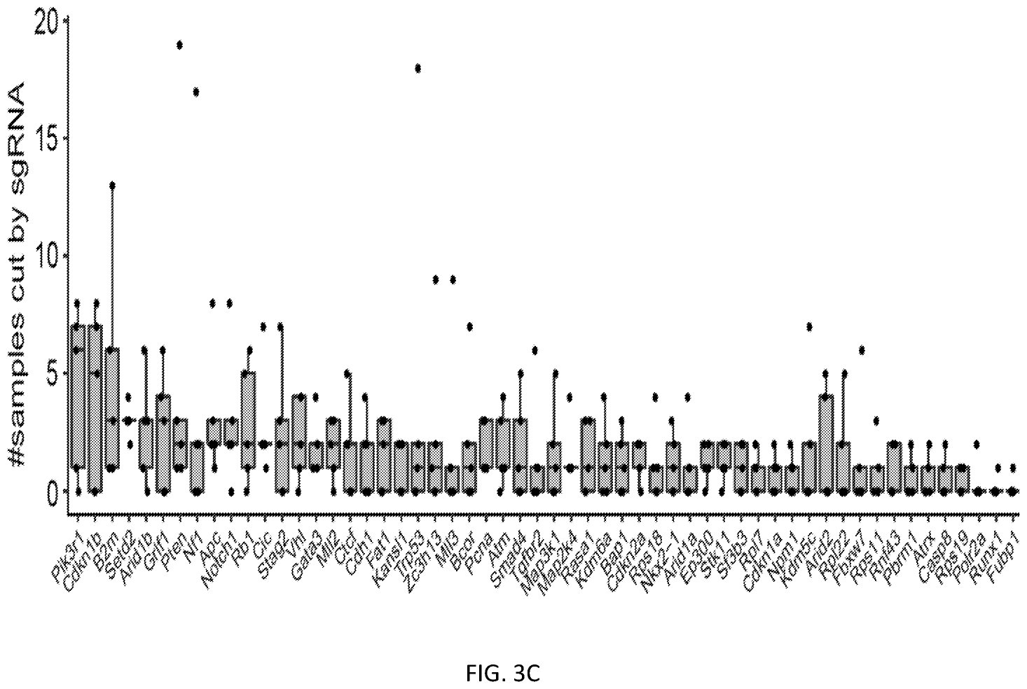

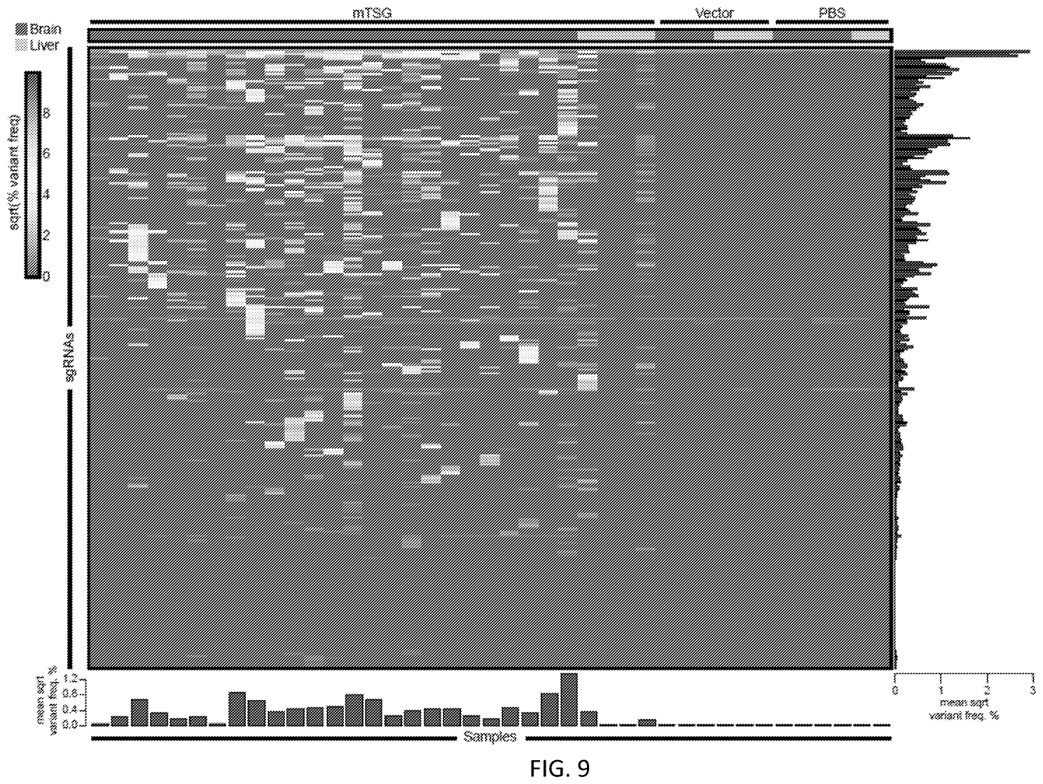

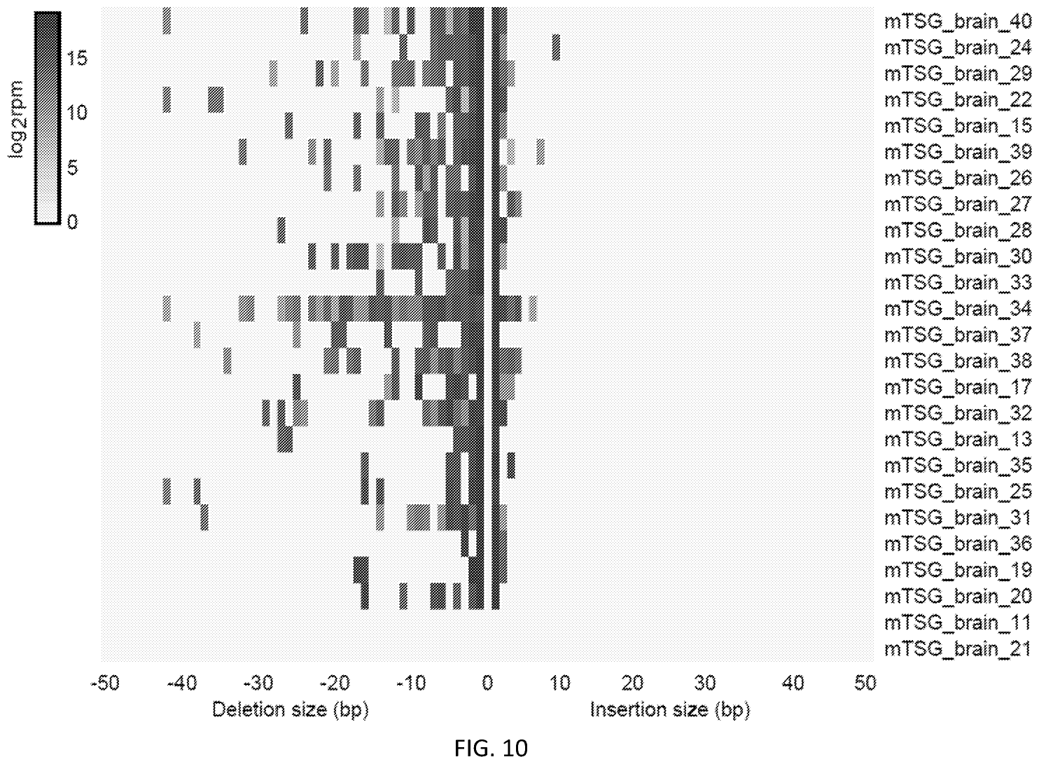

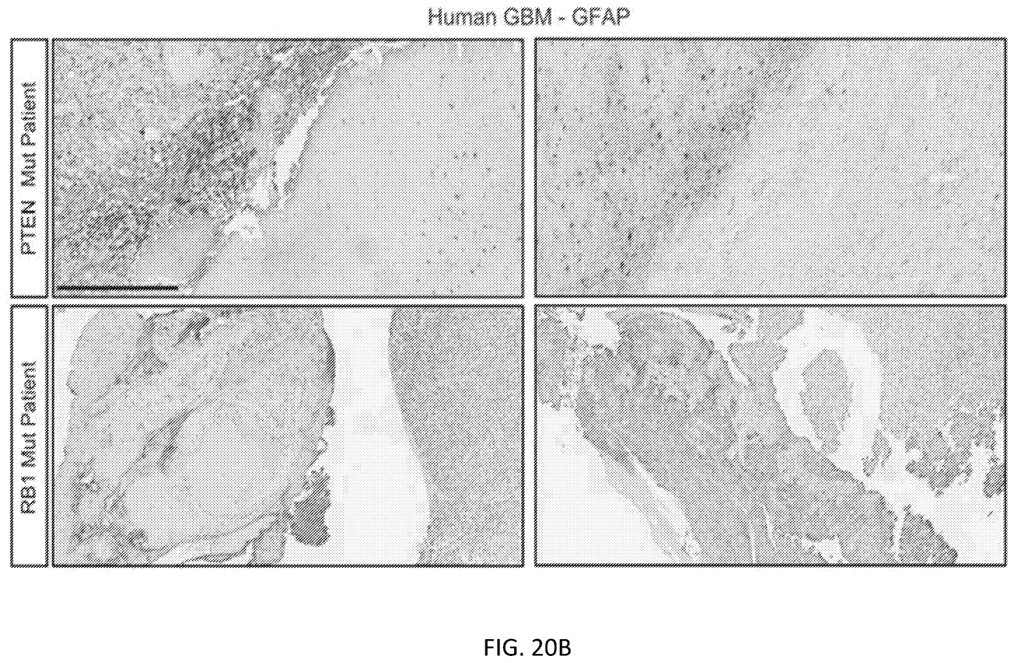

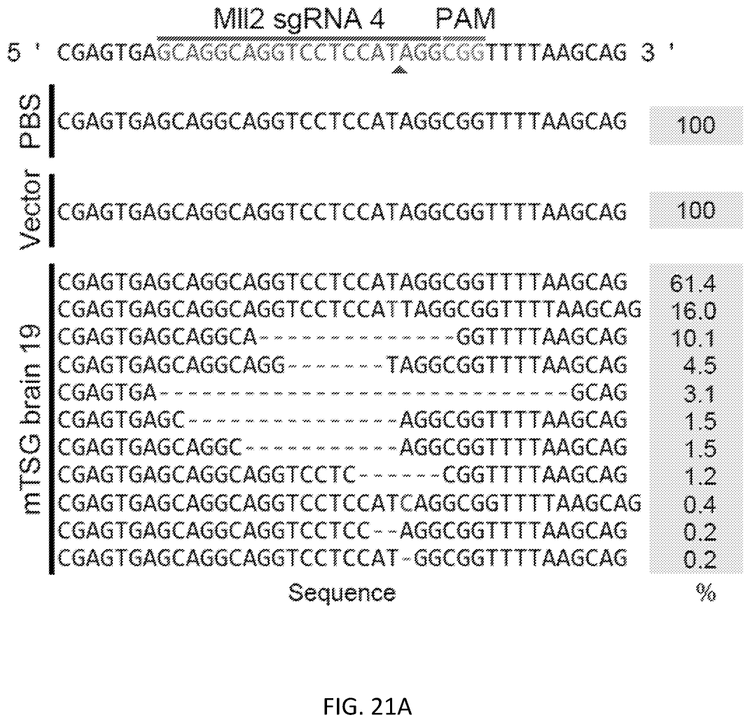

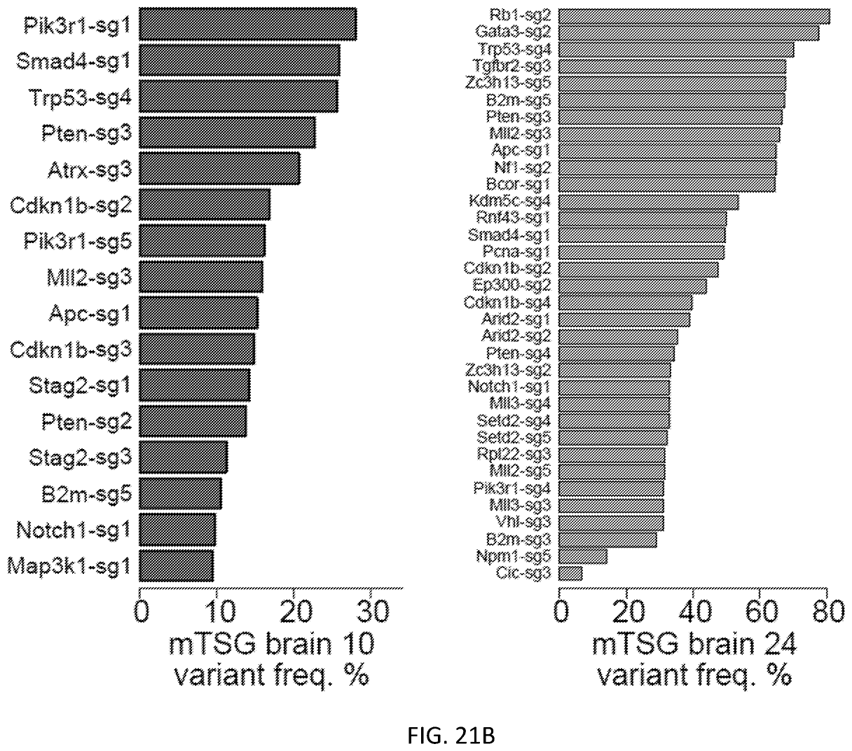

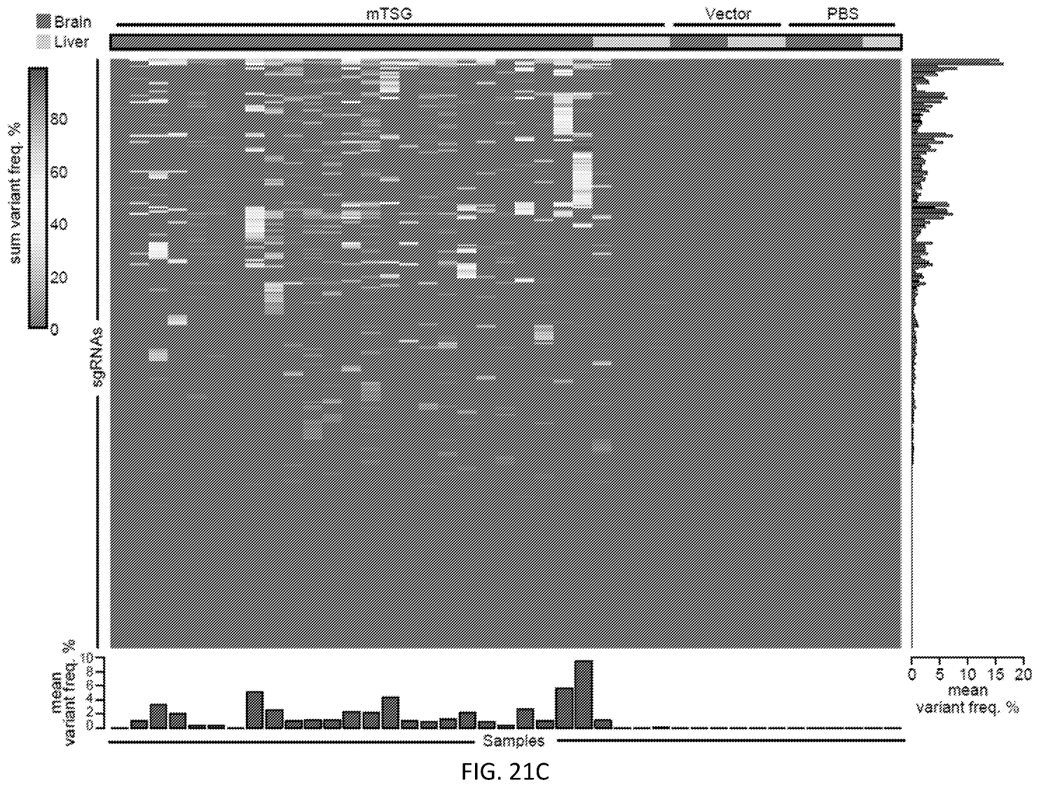

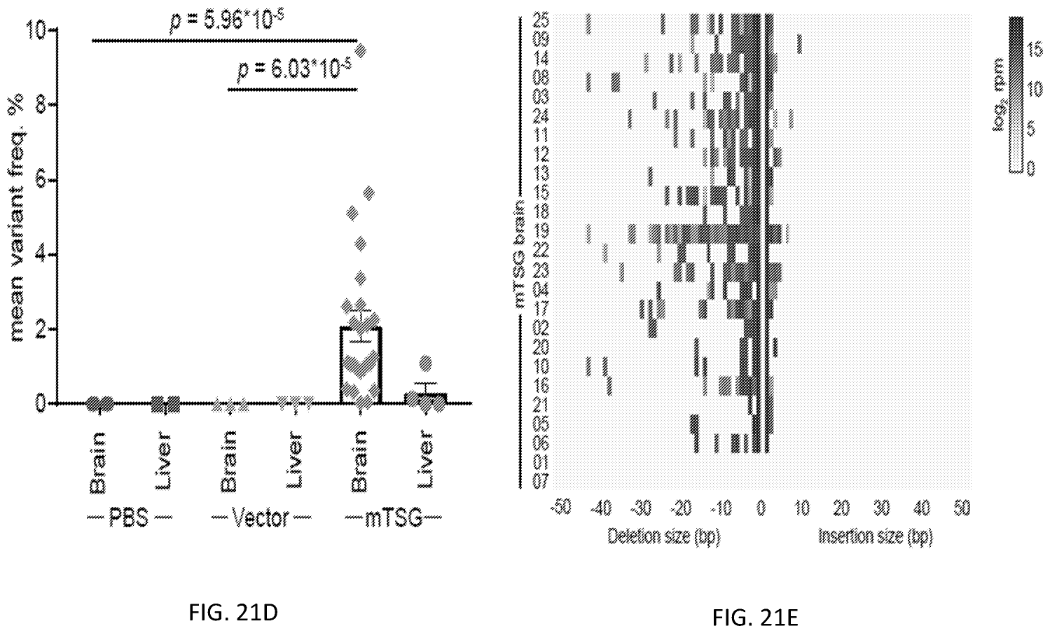

The mutant variants were analyzed at the predicted cutting sites of the 277 successfully captured sgRNAs across all samples. At the single sgRNA level, for example, at the predicted cutting site of sgRNA-4 in the Mixed-linkage leukemia protein 2 (Mll2) locus (also called histone-lysine N-methytransferase 2D, Kmt2d), various insertions and deletions (indels) were detected in AAV-mTSG but not AAV-vector or PBS mice (FIG. 3A; FIG. 21A). The indel frequencies were summed across all detected variants for each sgRNA target site in each sample, revealing a highly diverse pattern of variant frequencies generated by sgRNAs (FIG. 9). As shown in a meta-analysis of all variants of all sgRNAs for each sample, the predominant indels are deletions for virtually all samples (FIG. 10; Table 2). Most insertions are 1 bp in size (FIG. 10). The majority of these indels are out-of-frame (FIG. 10), disrupting the coding frame of the targeted proteins.

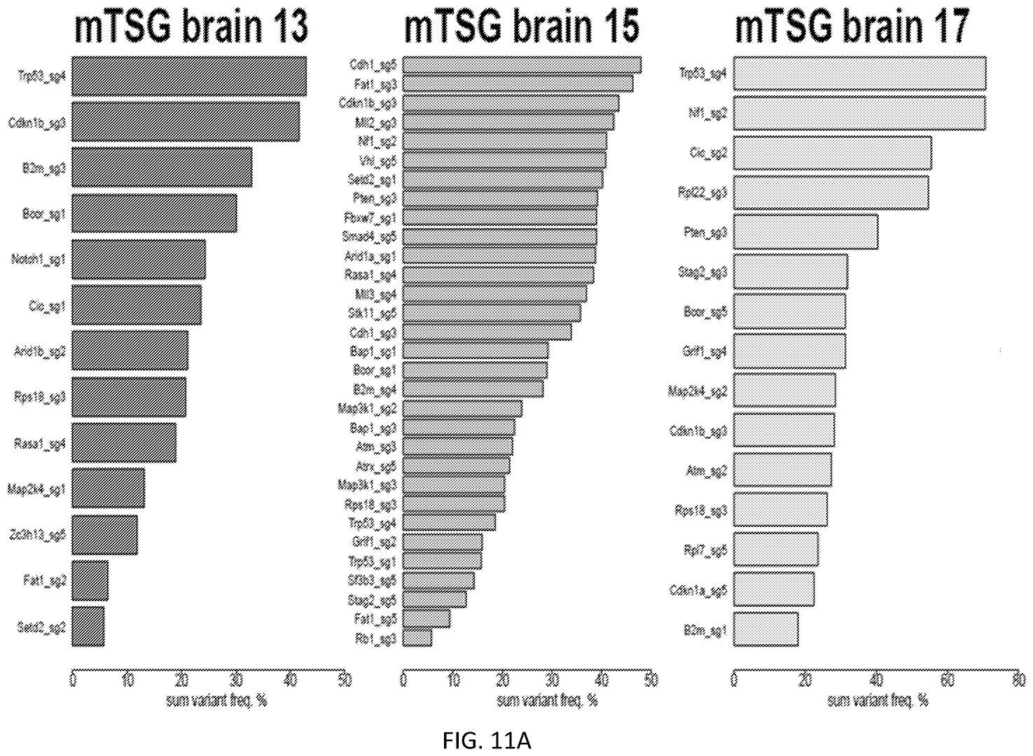

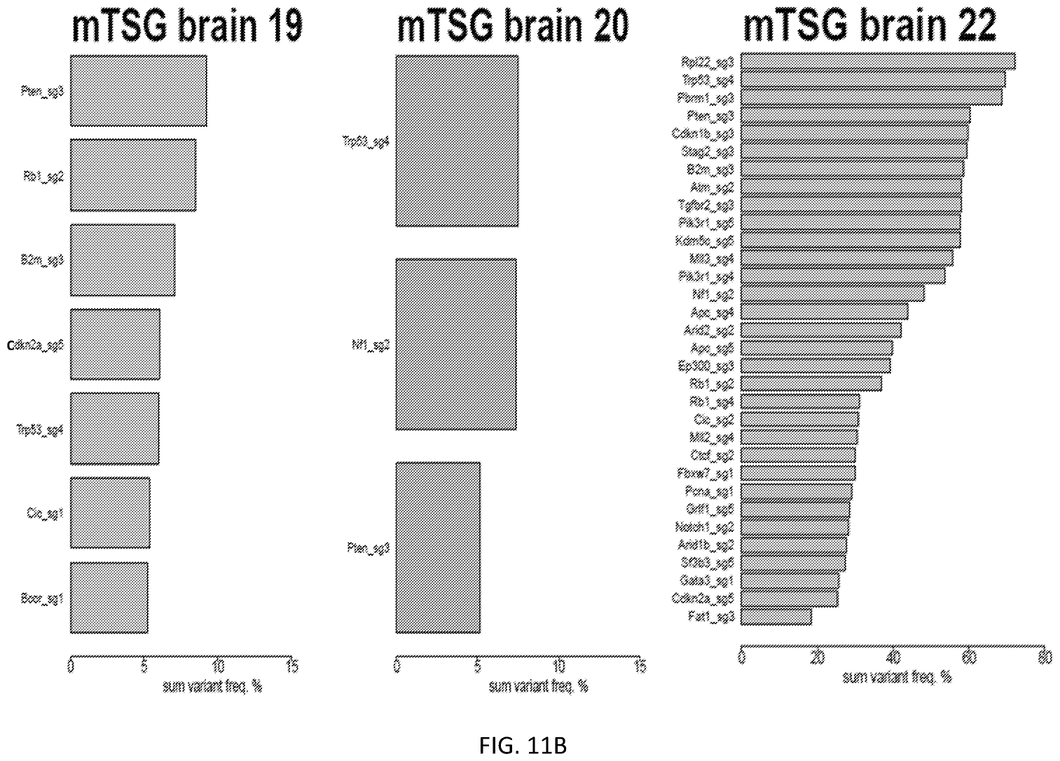

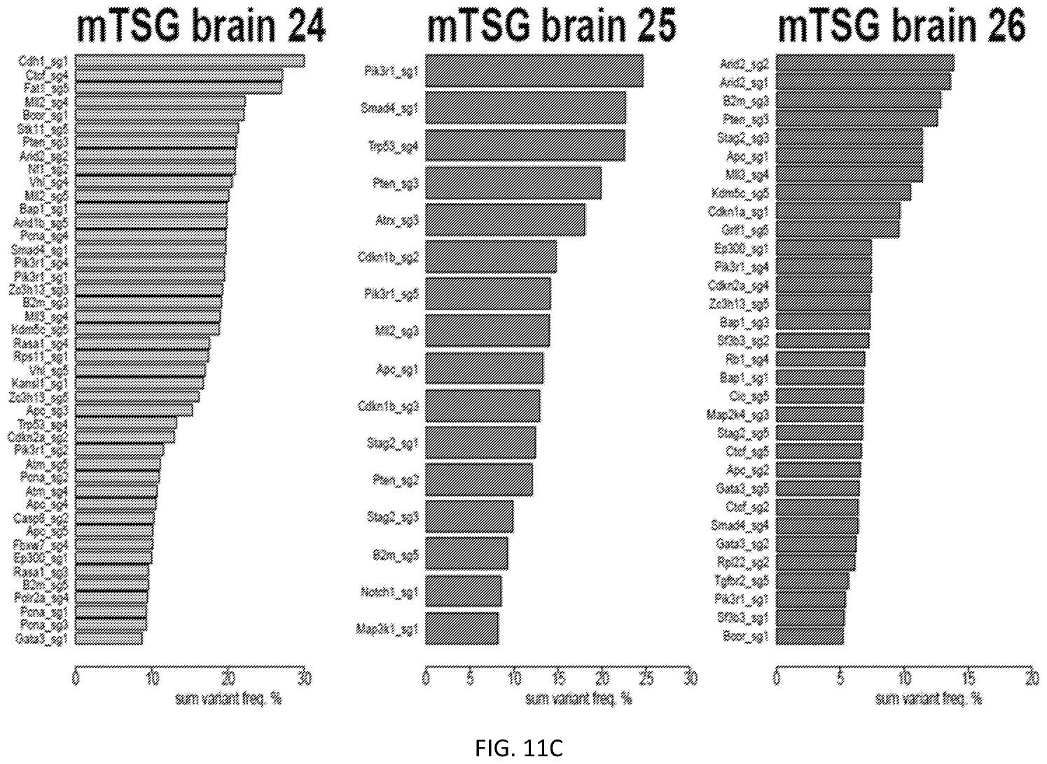

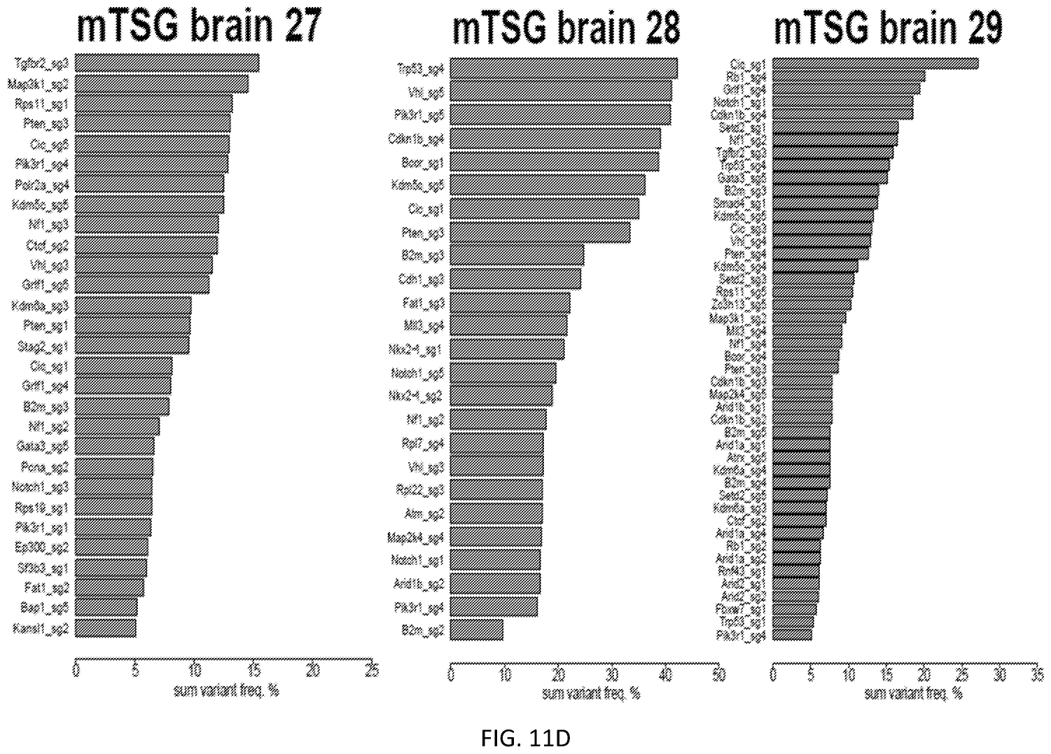

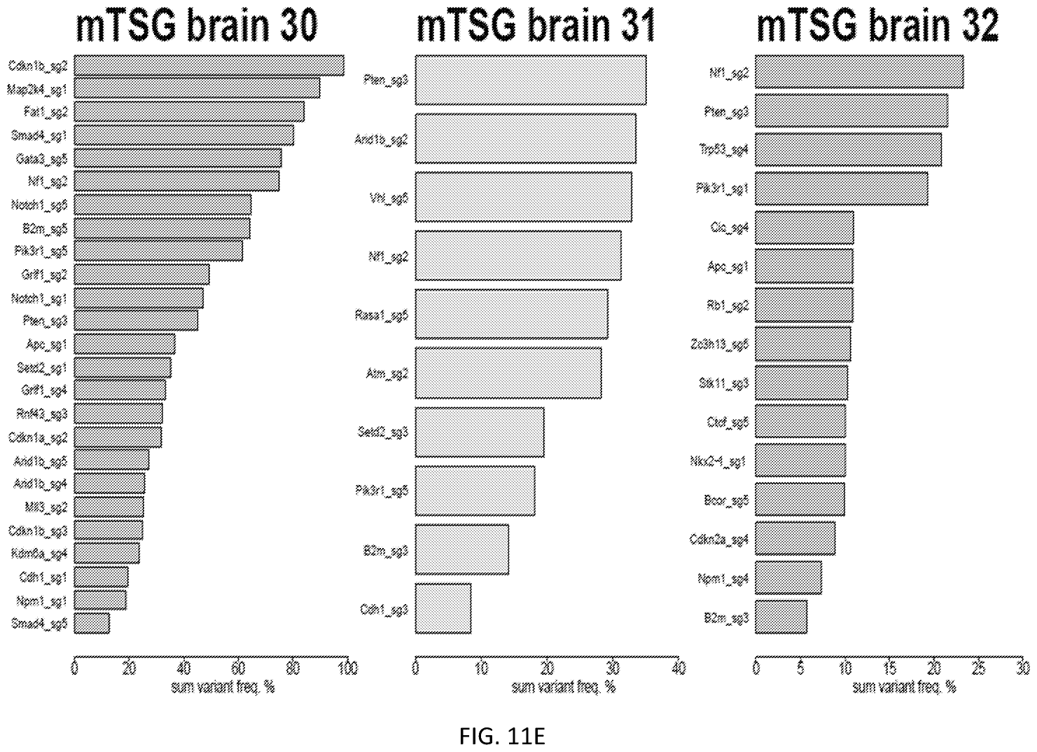

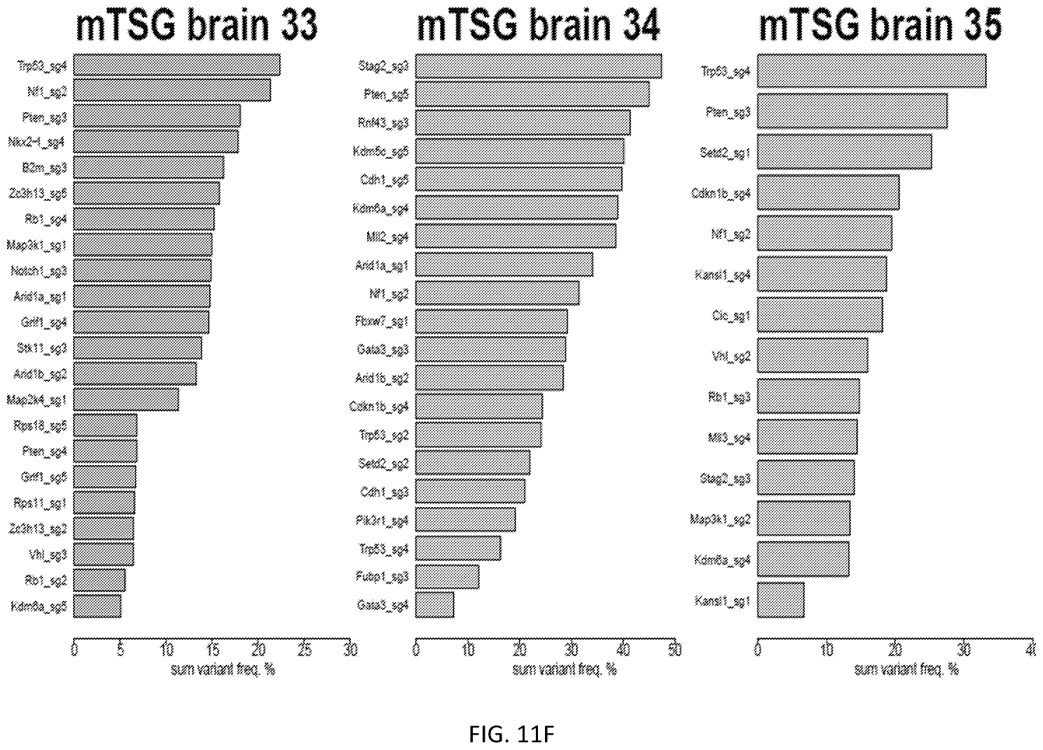

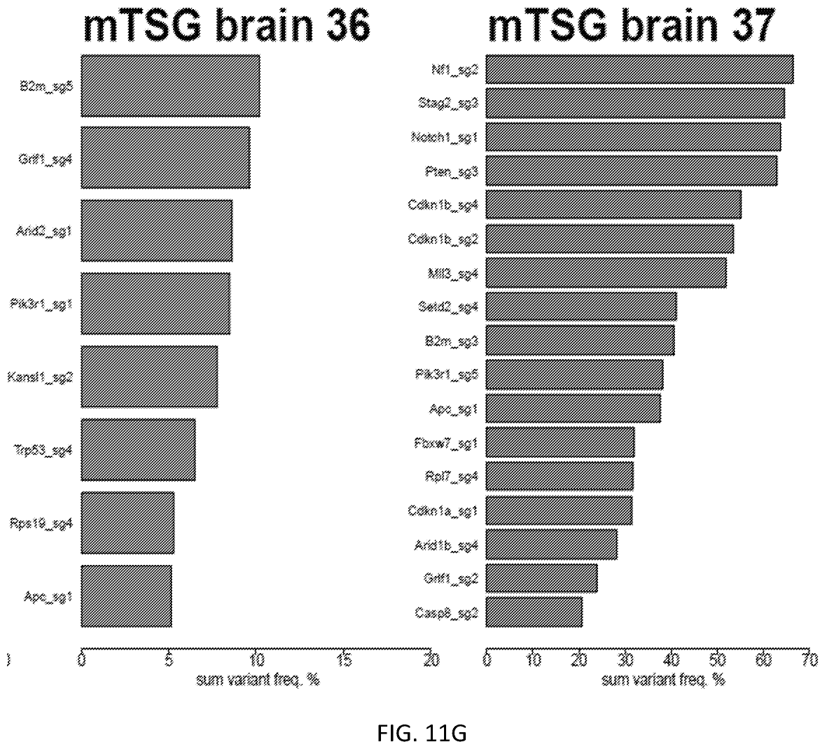

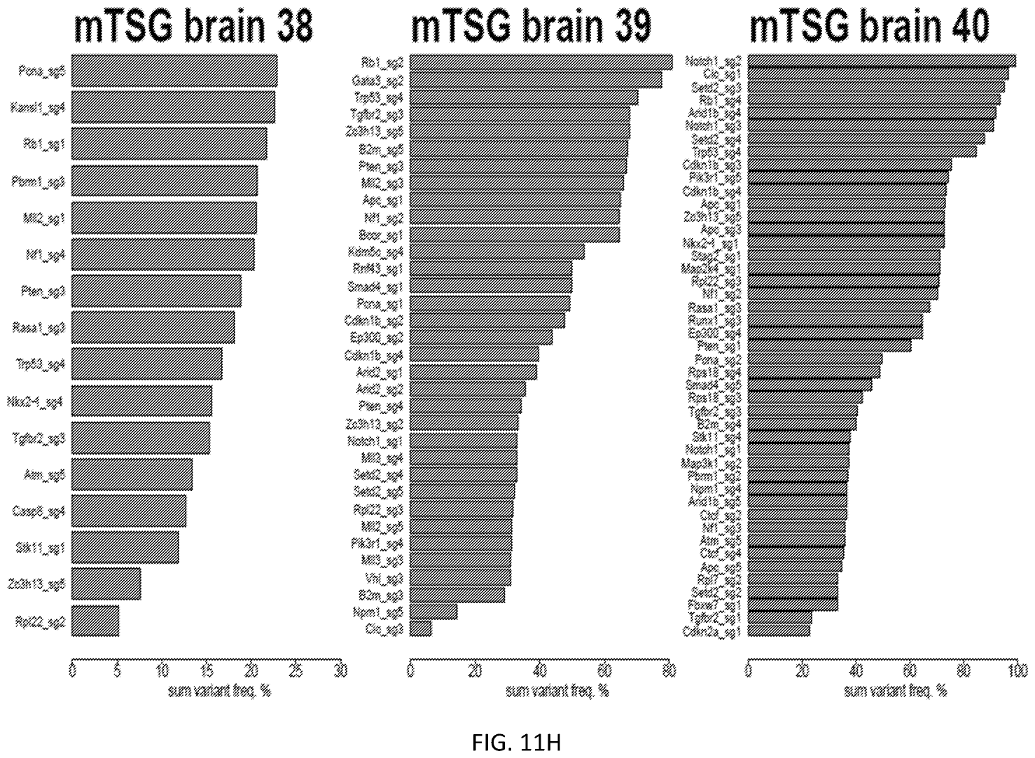

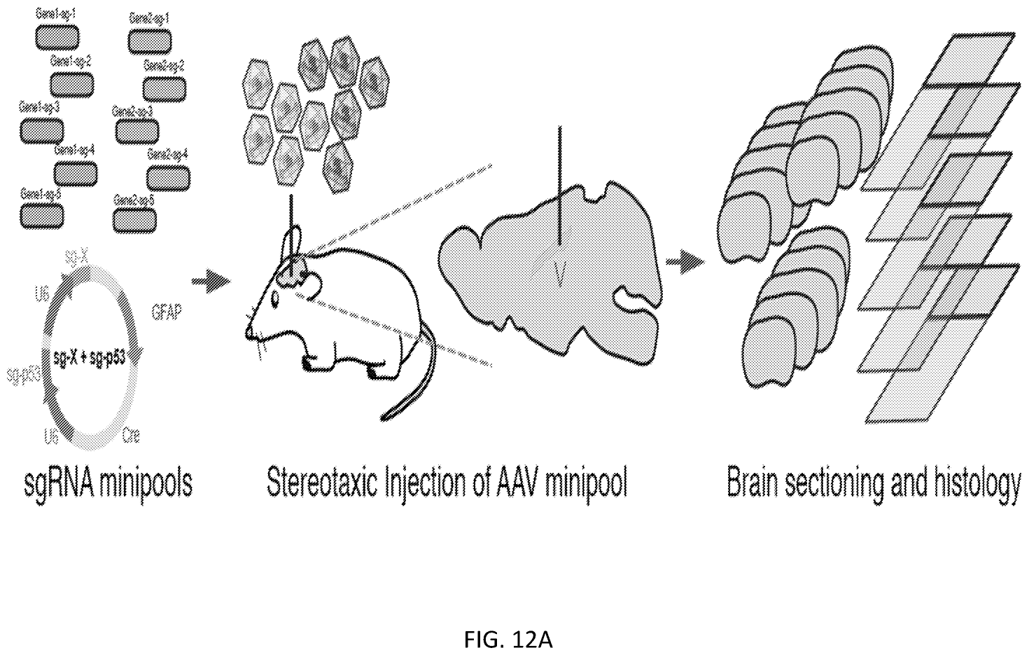

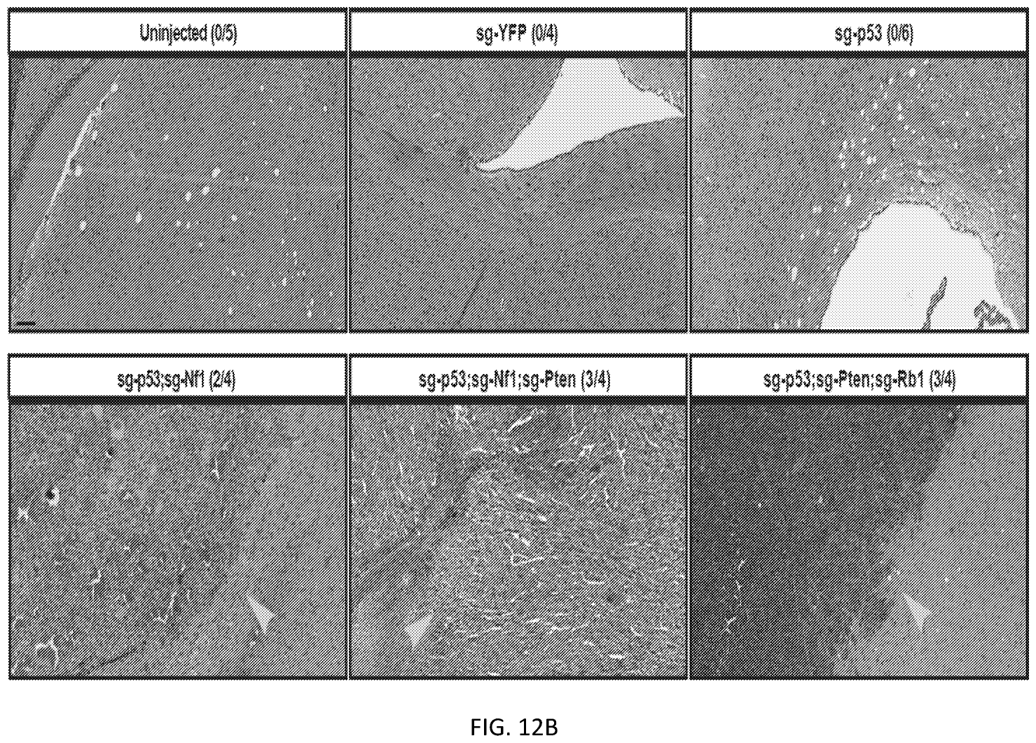

By applying a statistical analysis of mutational significance compared to samples from PBS and AAV-vector injected mice (false discovery rate (FDR)<= 1/12, or 8%), significantly mutated sgRNA sites (SMSs) were identified across all AAV-mTSG and AAV-vector samples (Table 4). Different mice showed radically different mutational landscapes (FIG. 3B; FIGS. 11A-11H). For example, one AAV-mTSG mouse (mTSG brain 25) had significant mutations at the predicted cutting sites of 16 out of 279 total gene-targeting sgRNAs in the mTSG library (FIG. 3B). These 16 SMSs were found in 12 different genes, and were subsequently defined as mouse significantly mutated genes (mSMGs)). A second example (mTSG brain 39) showed a more diverse mutational profile (34 SMSs for 26 mSMGs) (FIG. 3B). These data revealed that different driver mutations can be directly selected for during gliomagenesis. Across the 25 analyzed AAV-mTSG brains, the number of mice in which different sgRNAs were detected as an SMS varied tremendously (FIG. 3C), likely because same-gene-targeting sgRNAs might have differential capacity in generating mutations, or that different mutations in the same gene confer different selective advantages during GBM growth in mice.

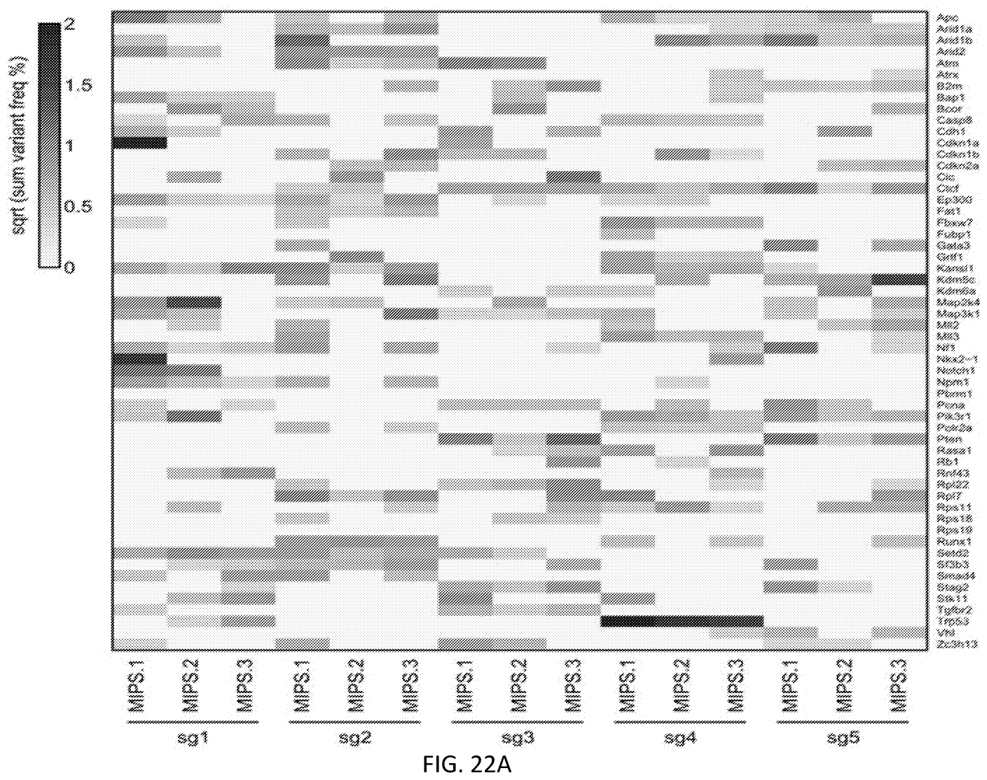

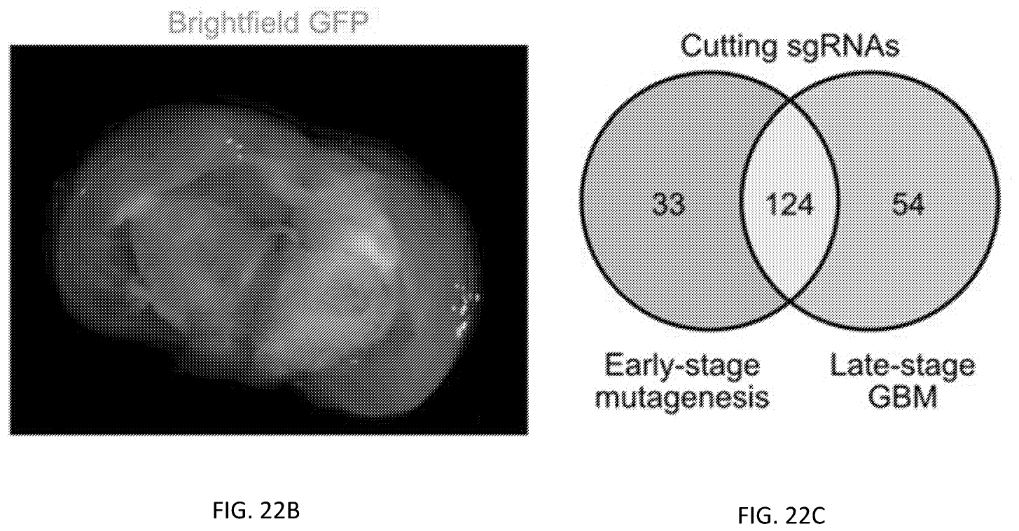

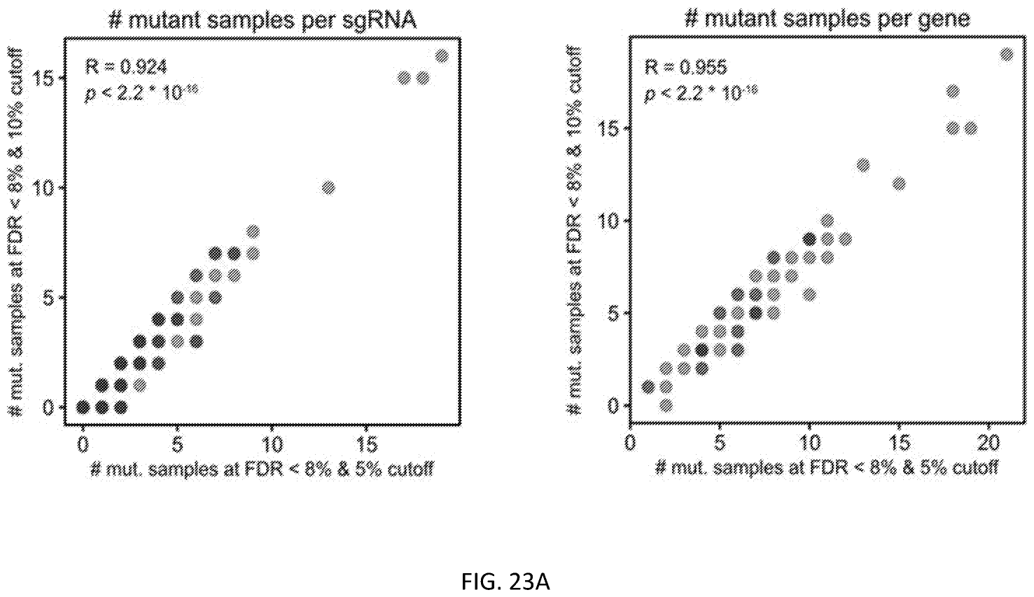

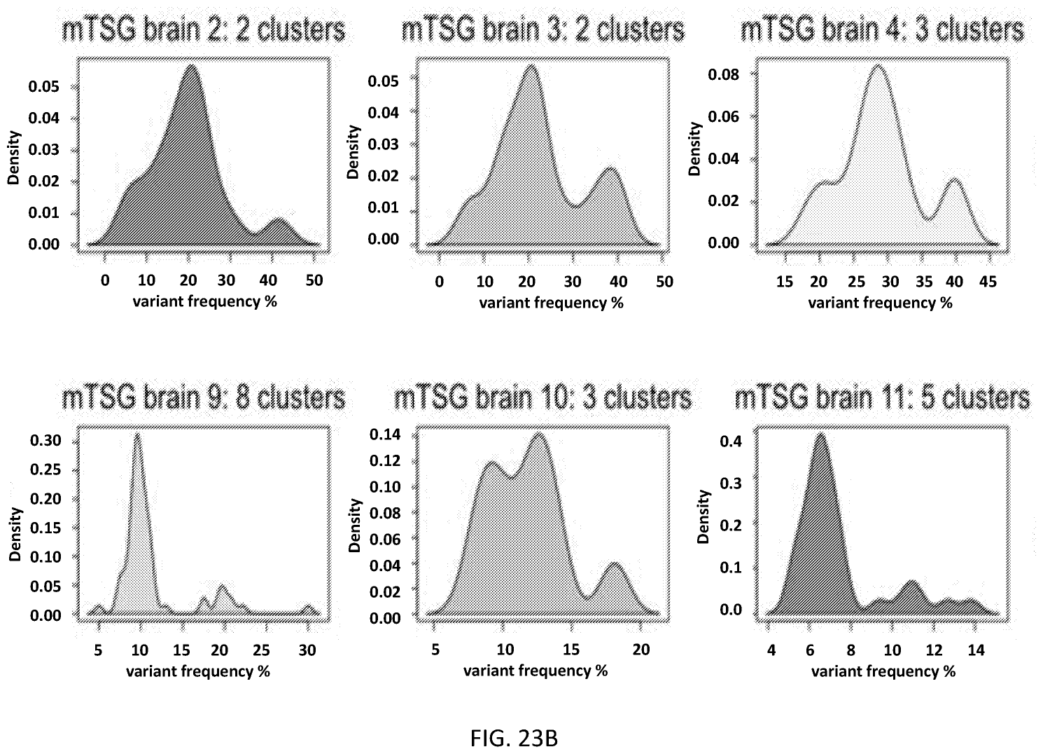

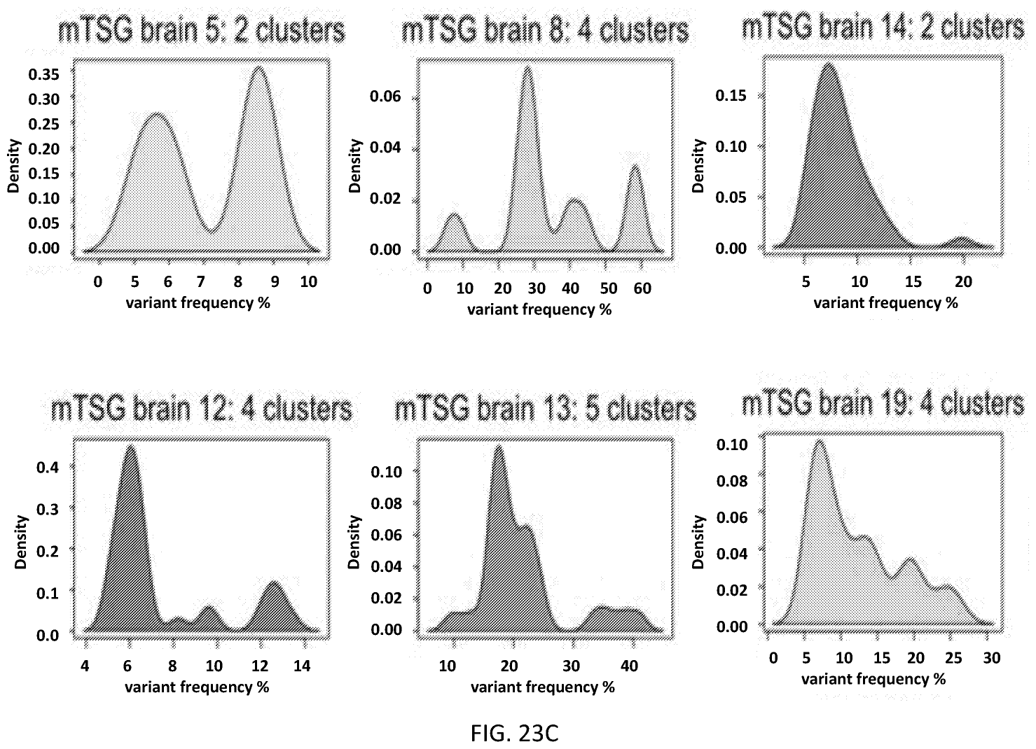

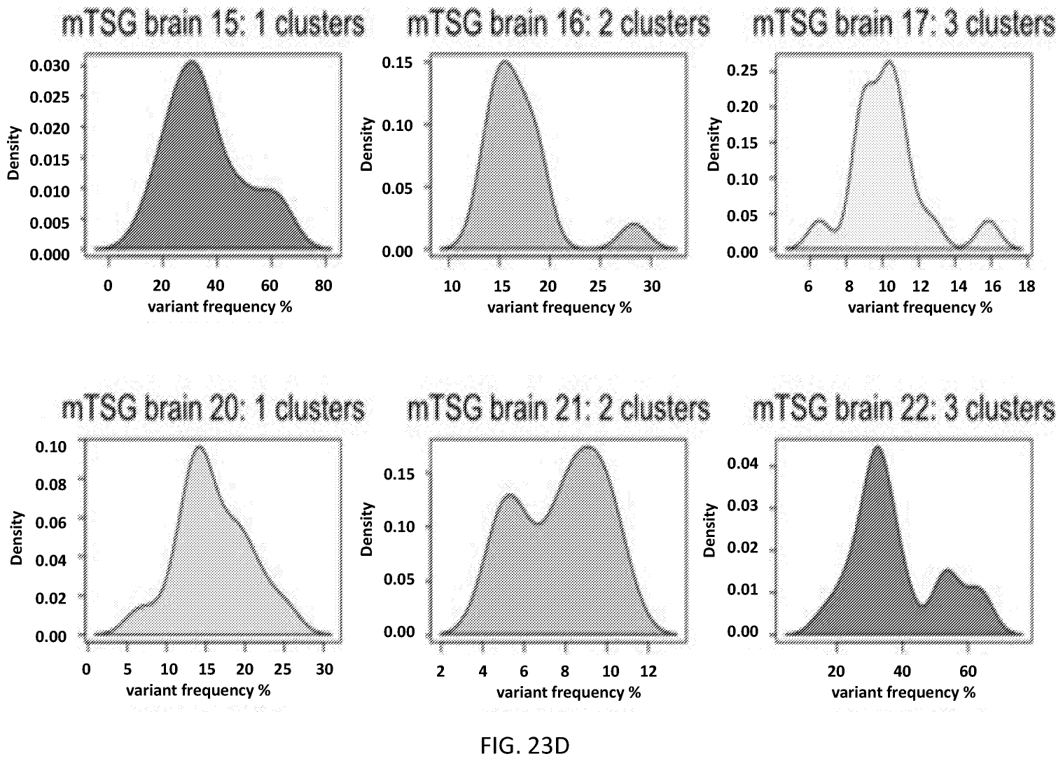

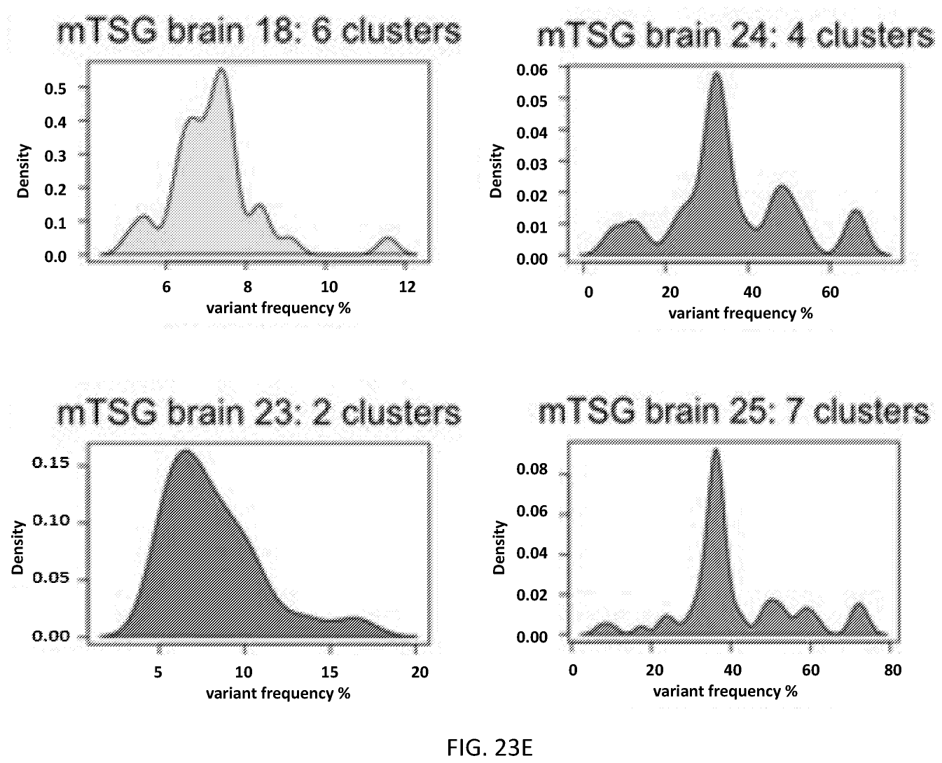

As gliomagenesis takes multiple months in mice (FIG. 1D), a survey of 3 mice at 3.5 weeks post-injection was conducted and capture sequencing performed to reveal early mutation profiles, as an approximation for in vivo sgRNA cutting efficiency (FIGS. 22A-22C). It was discovered that even lower-efficiency sgRNAs can end up being highly enriched in the process of tumorigenesis if the mutations they generated are strongly oncogenic (FIG. 22C; FIGS. 21B-21C). After removing germline variants, it was determined whether the regions flanking each sgRNA target site would be classified as significantly mutated sgRNA sites (SMSs). A false-discovery-rate (FDR) approach was implemented with (FDR< 1/12 PBS/vector samples, or 8%) as well as a flat 5% variant frequency cutoff. It was confirmed that the choice of alternative cutoffs did not alter the final SMS calls (FIG. 23A). With these criteria, a diverse mutational landscape was observed across most mice that were capture-sequenced (FIGS. 21B-21C, FIG. 11A-11H). As an example, one AAV-mTSG mouse (mTSG brain 10) had significant mutations at the predicted cutting sites of 16 out of 277 captured gene-targeting sgRNAs in the mTSG library, covering 12 significantly mutated genes (mSMGs) (FIG. 21B). A second example (mTSG brain 24) showed a more diverse mutational profile (34 SMSs for 26 mSMGs) (FIG. 21B). The raw indel frequencies were also summed across all detected variants for each sgRNA target site in each sample, revealing a highly diverse pattern of variant frequencies generated by this sgRNA pool (FIG. 21C). Comparing brain samples between treatment groups, AAV-mTSG injected brains had significantly higher mean variant frequencies (2.087±0.429 s.e.m., n=25) compared to vector (0.005±0.001, n=3) or PBS (0.003±0.001, n=4) injected brains (two-tailed Welch's t-test, t₂₄=4.85 and t₂₄=4.86, p=6.03*10⁻⁵ and p=5.96*10⁻⁵ for mTSG vs. vector and mTSG vs. PBS, respectively) (FIGS. 21C-21D). Comparing targeted vs. non-targeted organs in AAV-mTSG injected mice, the mean variant frequencies of brains (2.087±0.429, n=25) were significantly higher than livers (0.309±0.261, n=4) (two-tailed Welch's t-test, t_{21.48}=3.54, p=0.002) (FIGS. 21C-21D). The predominant indels were deletions for virtually all samples, and most insertions at SMS sites were 1 bp in size (FIG. 21E). Distinct variant frequency clusters of sgRNA-induced indels were identified that may serve as an approximation to the clonality of these tumors. From this analysis, only 2/22 of the brains had single-cluster tumors, with the majority ( 20/22) being comprised of multiple clusters (FIG. 23B-23E). These data demonstrate on-target, pooled genome editing in the brain at a library scale, stochastically generating loss-of-function mutations in native glial cells and priming them for selection during gliomagenesis.

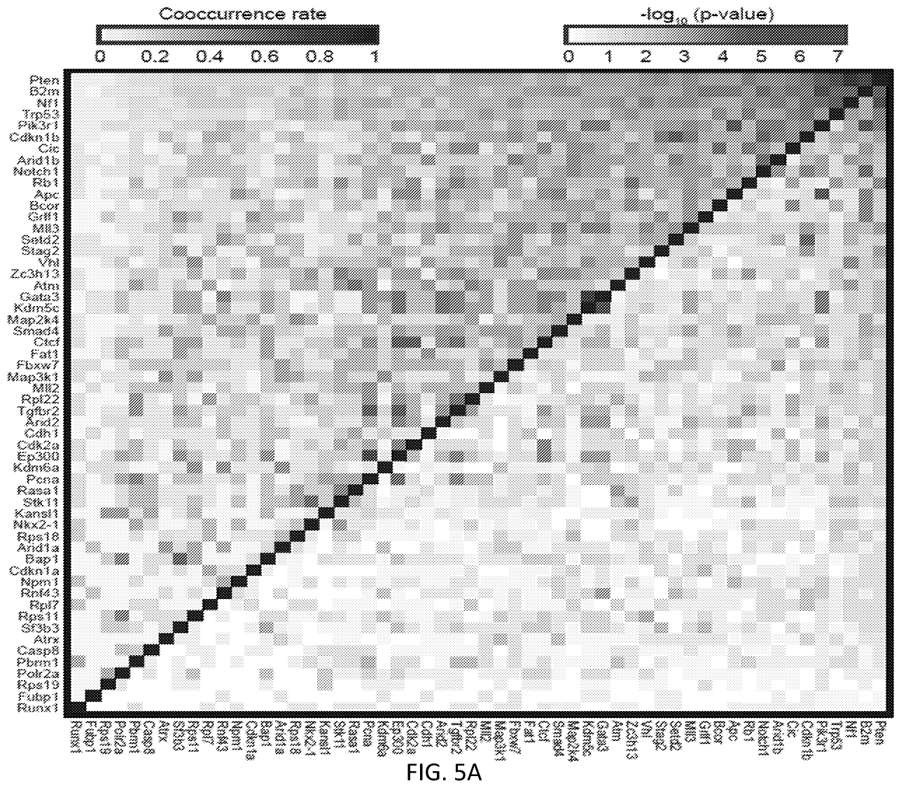

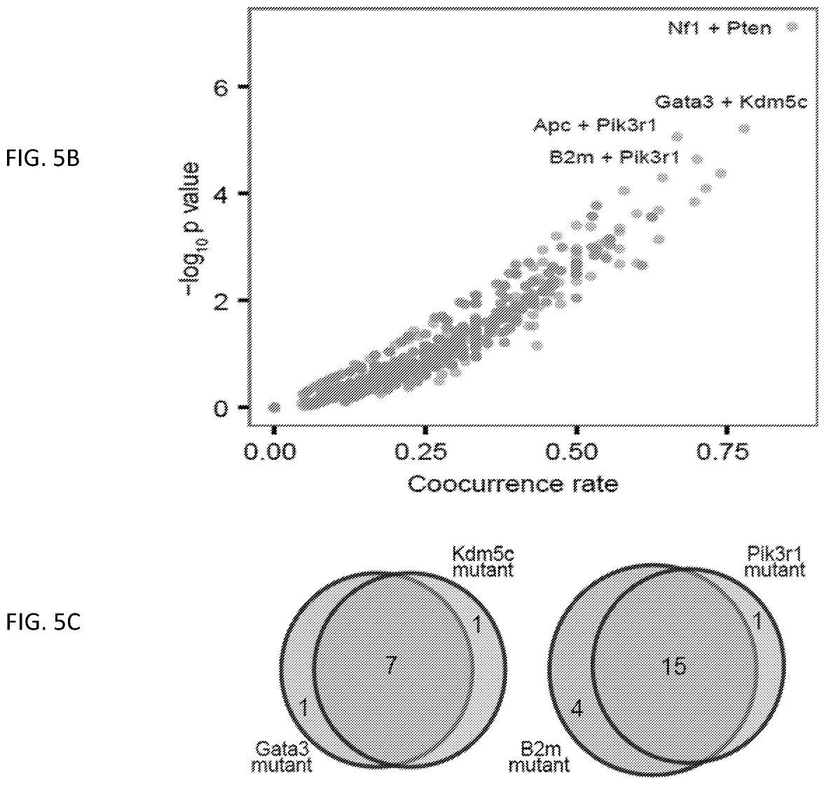

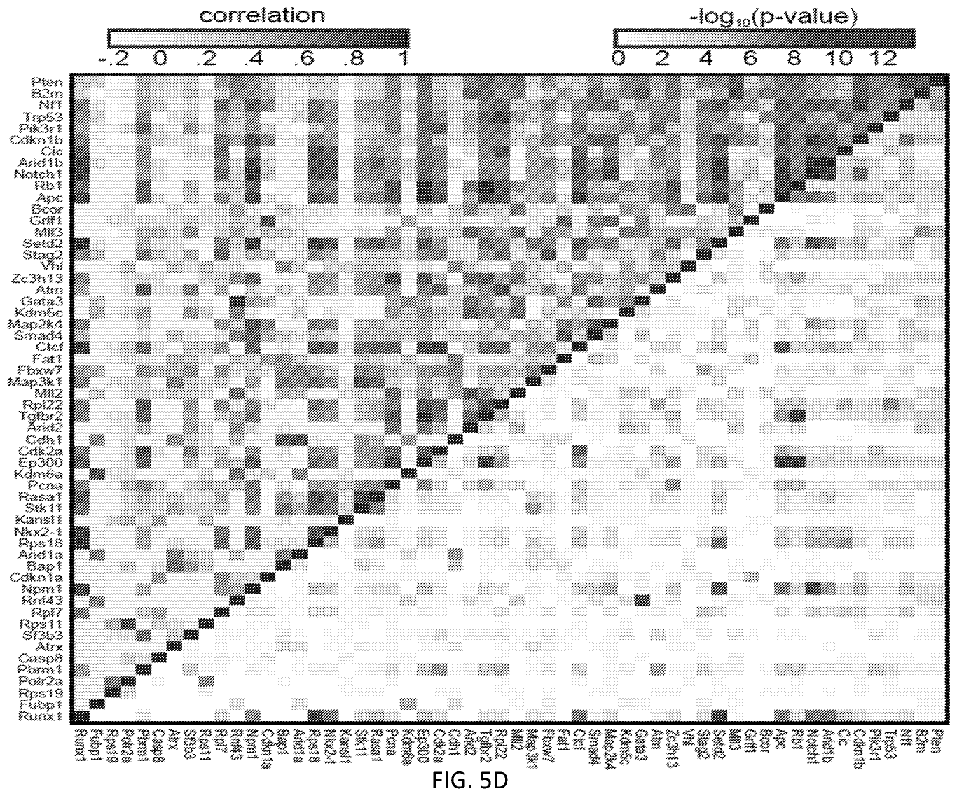

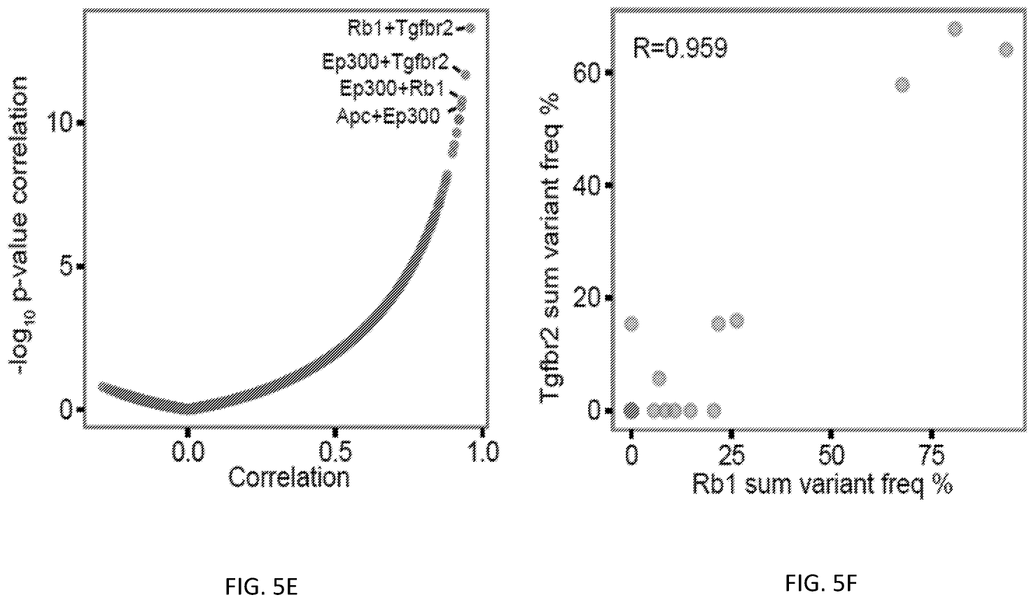

The mutational data from the SMS level to the mSMG level was summarized, and co-mutation analysis of this functional map of tumor suppressors was performed in mouse GBM. The number of SMSs ranged from 3 to 46, with mSMGs ranging from 3 to 33 (excluding the two samples with zero SMSs). A pairwise map of co-occurrence of mSMGs was generated, and the number of double-mutant samples in each pair of genes calculated, as well as the statistical significance of their co-occurrence compared to random chance (FIG. 5A). The Nf1/Pten pair emerged as the top pair in both number of co-occurrence and statistical significance ( 20/25=80% of mice, hypergeometric test, p<1 e-7), reminiscent of this gene pair being strong tumor suppressors of GBM in human. Several previously undocumented combinations emerged with high numbers of co-occurrence and statistical significance, such as Gata3/Kdm5c, Apc/Pik3r1 and B2m/Pik3r1 (FIG. 5B). For instance, either Gata3 or Kdm5c alone is significantly mutated in 32% ( 8/25) of mice but they co-occurred ⅞ times (87.5%) (hypergeometric test, p<1e-5) (FIG. 5C).

In addition, correlation analysis of mutant frequencies of each pairs of tumor suppressors across all individual mice was performed (FIG. 5D). 32% ( 495/1540) of the gene pairs positively correlate with each other (Pearson correlation>0, p-value of t-statistic of correlation coefficient<0.05), with the remaining pairs being insignificant (FIGS. 5D-5E). The most significantly correlated gene pairs are Rb1/Tgfbr2 (correlation=0.959, p=4.83e-14) (FIGS. 5E-5F; FIG. 15), Ep300/Tgfbr2 (correlation=0.942, p=2.08e-12), Ep300/Rb1 (correlation=0.931, p=1.63e-11), Apc/Ep300 (correlation=0.927, p=2.71e-11). These data reveal systematic co-occurrence and correlation relationships of mutant variants during glioblastoma progression in vivo.

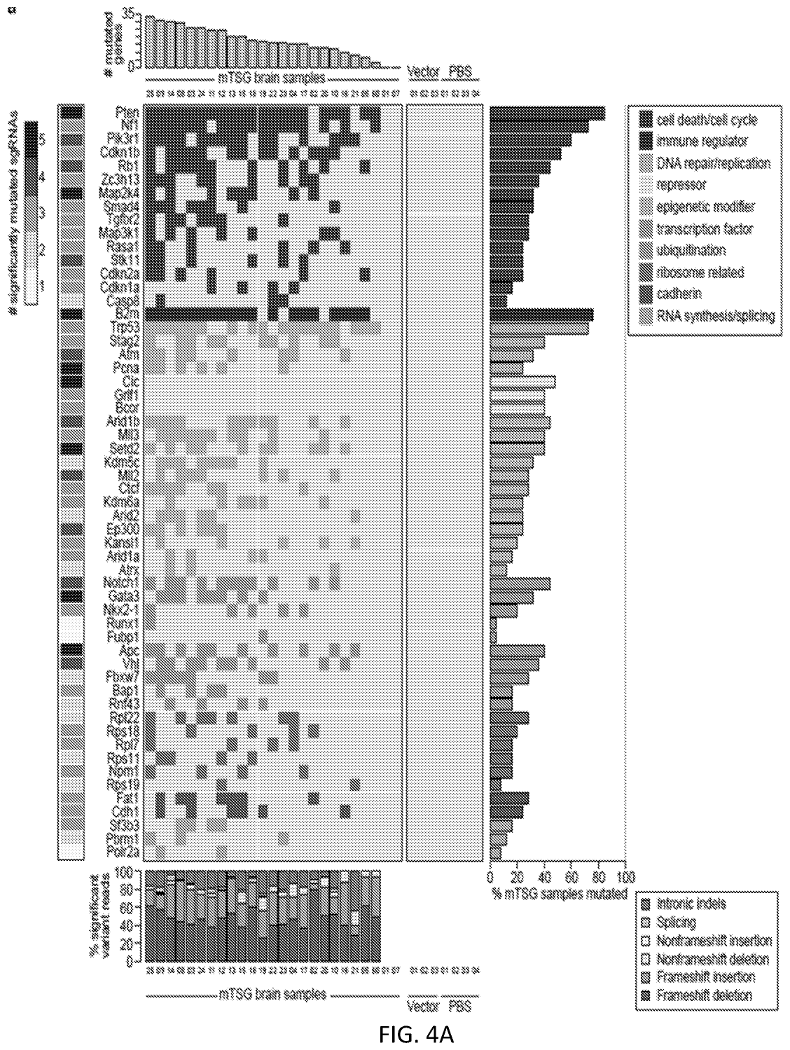

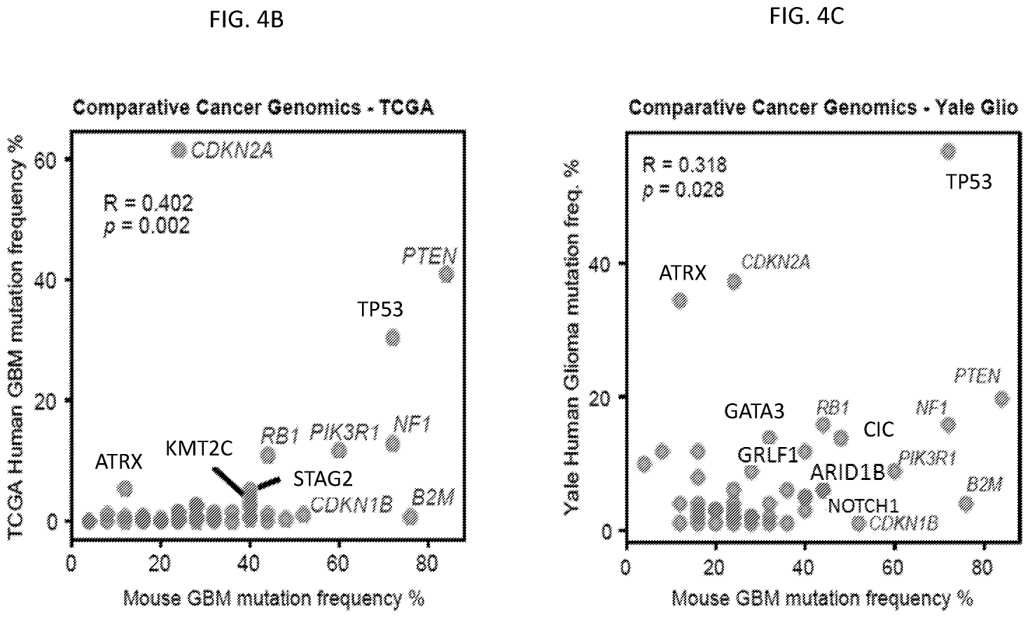

The mutational data was summarized from the SMS level to the mSMG level and an oncomap of all mTSG brain samples was created (FIG. 4A). Although two mice had no detected SMGs, across all mice with SMGs ( 23/25), the detected variants were predominantly frameshift indels (frameshift reads/total variant fraction>60% in 22/23 mice), compared to non-frameshift indels, splicing indels and intronic (FIG. 4A, bottom panel). Surprisingly, all 56 genes have at least one SMSs, with eight of them having all 5 sgRNAs being called as SMSs (FIG. 4A left and middle panel), including Phosphotase and tensin homolog (Pten), Mitogen-Activated Protein Kinase Kinase 4 (Map2k4), Beta-2 microglobulin (B2m), Proliferating Cell Nuclear Antigen (Pcna), Capicua Transcriptional Repressor (Cic), SET Domain Containing 2 (Setd2), GATA Binding Protein 3 (Gata3) and Adenomatous Polyposis Coli (Apc). Across all mSMGs in the 25 AAV-mTSG mouse brains, Pten is the top most frequently mutated ( 21/25=84% of mice), with all 5 sgRNAs being called as SMSs. B2m, a core component of major histocompatibility complex (MHC) class I that is essential for antigen presentation, appeared as the second most frequently mutated ( 19/25=76% of mice) (FIG. 4A, right panel). The mSMGs encompass functionally diverse categories of proteins, including cell death or cell cycle regulators, with highly mutated examples such as Pten, Neurofibromatosis 1 (Nf1), Phosphoinositide-3-Kinase Regulatory Subunit 1 (Pik3r1), Cyclin-Dependent Kinase Inhibitor 1b (Cdkn1b) and Retinoblastoma 1 (Rb1); immunological regulator B2m; DNA repair and replication regulators Trp53, Pcna, Stromal Antigen 2 (Stag2) and Ataxia Telangiectasia Mutated (Atm); repressors Cic and Bc16 Corepressor (Bcor); epigenetic regulators Setd2, AT-Rich Interaction Domain 1B (Arid1b), and Mixed linkage leukemia protein 3 (Mll3); transcription factors Gata3 and Notch1; cadherin type proteins Cadherin 1 (Cdh1) and FAT Atypical Cadherin 1 (Fad); as well as ubiquitin ligases Apc, Von Hippel-Lindau Tumor Suppressor (Vhl) and F-Box And WD Repeat Domain Containing 7 (Fbxw 7) (FIG. 4A). Many of the genes were significantly mutated in 20% to 50% of mice, with most of the epigenetic regulators in this range, such as Arid1b, Mll3, Setd2, Mll2, Kdm5c, Kdm6a, Arid2 and Ctcf (FIG. 4A), highlighting the role of epigenetic regulators in brain tumorigenesis. This analysis revealed, in a quantitative manner, the relative phenotypic strength of specific loss-of-function mutations in driving gliomagenesis in vivo.

Six mSMGs were mutated in more than half of the mice, including Pten, Nf1, Pik3r1, Cdkn1b, B2m, and Trp53 (FIGS. 5A-5F). Trp53 is significantly mutated in the vast majority of mice ( 18/25=72%). Of note, a Trp53 sgRNA exists in the backbone vector, but not all mice presented with Trp53 as an mSMG, likely due to the dynamics of selection in vivo so that not all Trp53 mutant cells became the final dominant tumor clones. Surprisingly, the 7 annotated essential genes selected as internal controls, including Splicing Factor 3b Subunit 3 (Sf3b3), RNA polymerase II subunit A (Po1r2a) and ribosomal protein encoding genes Rp122, Rps18, Rpl7, Rps11, Rps19, were found mutated in 10% to 30% of mice, although with relatively smaller number of SMSs per gene (control gene set, n=7, average 2.28±0.286 SMSs/gene; TCGA TSG set, n=49, average 3.26±0.153 SMSs/gene; two-sided t test, p=0.026) (FIGS. 5A-5F). While variants of these genes might be passenger mutations or might have off-target effects, the possibility of them being functionally selected during tumorigenesis cannot be ruled out, as Polr2a, certain splicing factors and some ribosomal protein subunits have recently been found as SMGs in several types of cancers with functional evidence of them being regulators of tumorigenesis or cancer cell growth. Many of the mSMGs are significantly mutated in 20% to 50% of mice, with most of the epigenetic regulators in this range, such as Arid1b, Mll3, Setd2, Mll2, Lysine Demethylase 5C (Kdm5c), Lysine Demethylase 6A (Kdm6a), AT-Rich Interaction Domain 2 (Arid2) and CCCTC-Binding Factor (Ctcf) (FIGS. 5A-5F). Altogether, the integrative approaches demonstrated herein revealed a high-throughput functional map of tumor suppressors in GBM.

Mutational frequencies in mice were compared to the variant frequencies of their homologous genes in human GBM with their frequencies of non-silent mutation and deletion. For these 56 genes, the mutation frequencies in mouse GBMs (an end-product of pooled mutagenesis and in vivo gliomagenesis) significantly correlated with the mutation frequencies in TCGA GBM patients (Pearson correlation R=0.402, p=2.1*10⁻³) (FIG. 4B). To further investigate this correlation, clinical cancer genomics data from the Yale Glioma tissue bank, a source independent from TCGA was utilized. Collectively, the mouse mutation frequencies again significantly correlated with those in human patients (R=0.318, p=0.0277) (FIG. 4C). These data suggest that the AAV-CRISPR autochthonous GBM mouse model revealed a quantitative phenotypic profile of tumor suppressors reflecting the genomic landscape of human GBM patients.

### Example 3: Co-Mutation Analysis Identifies Frequently Co-Occurring Driver Combinations

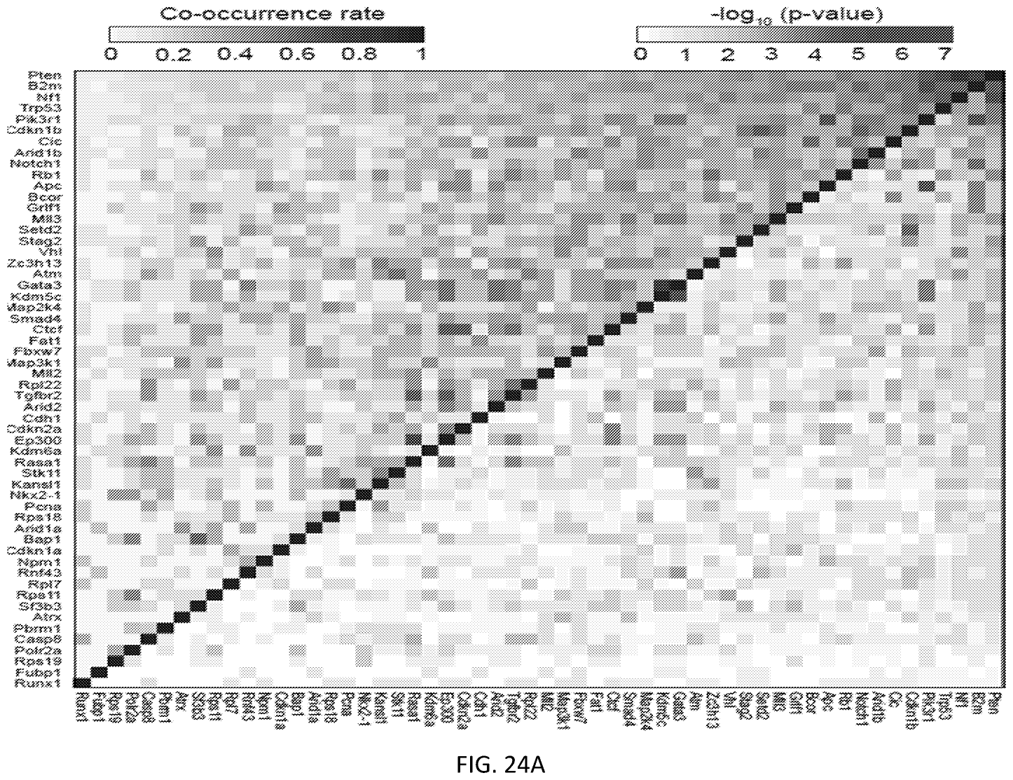

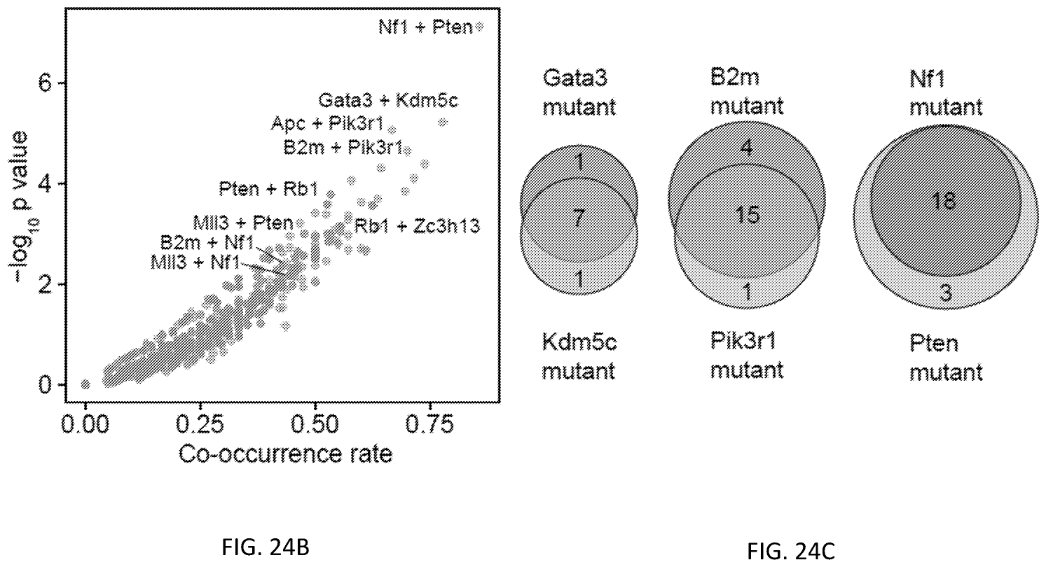

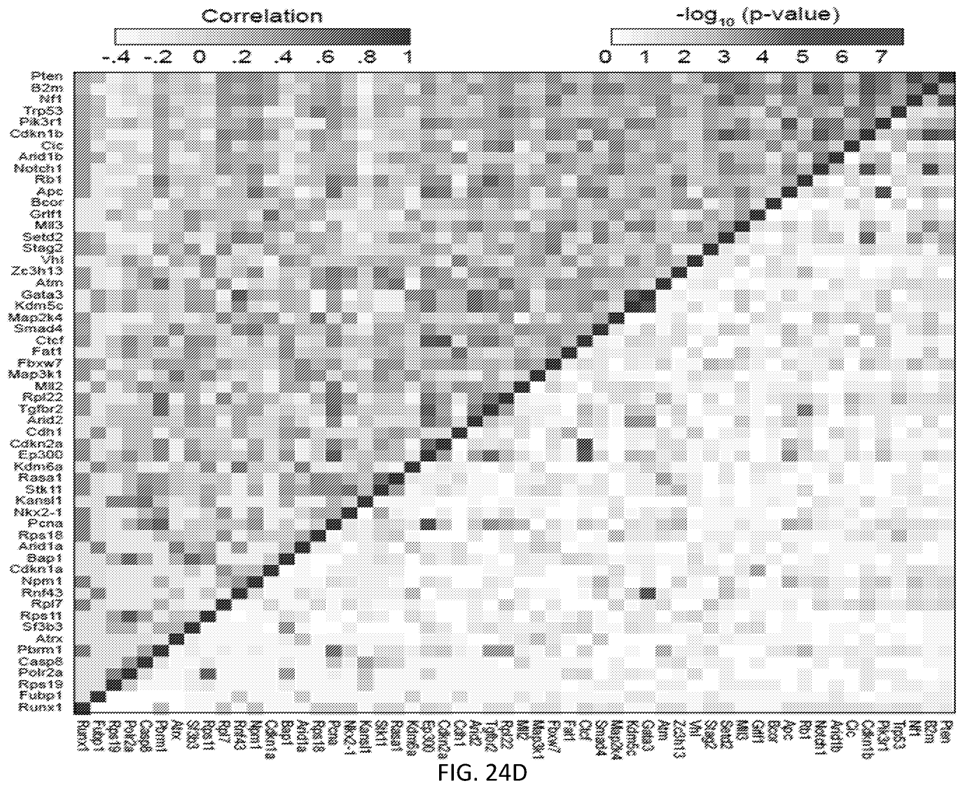

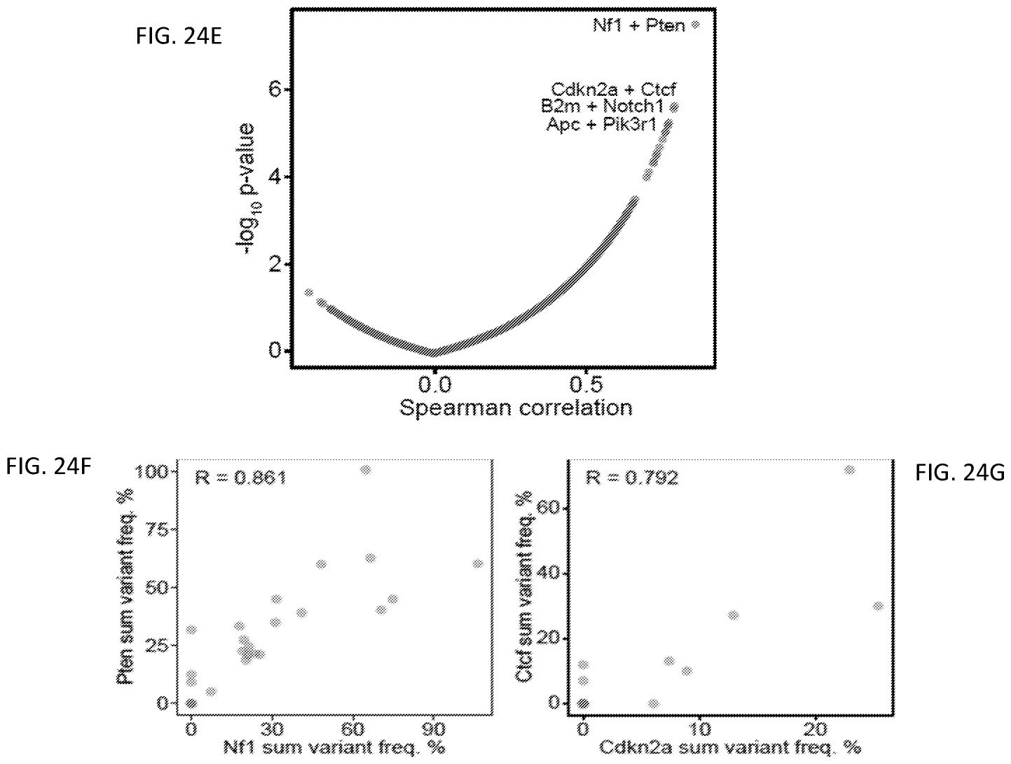

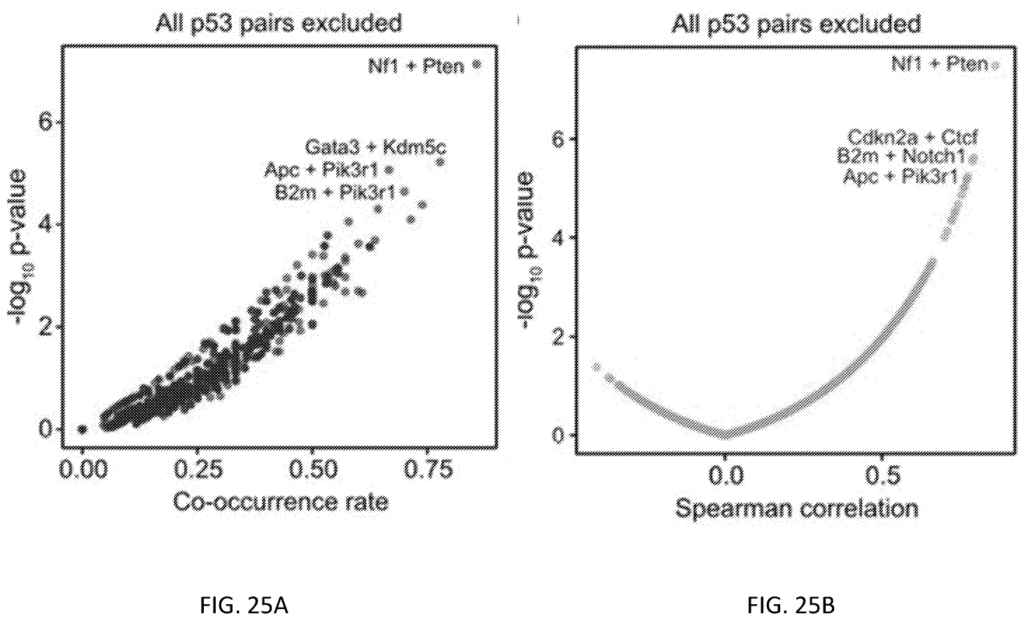

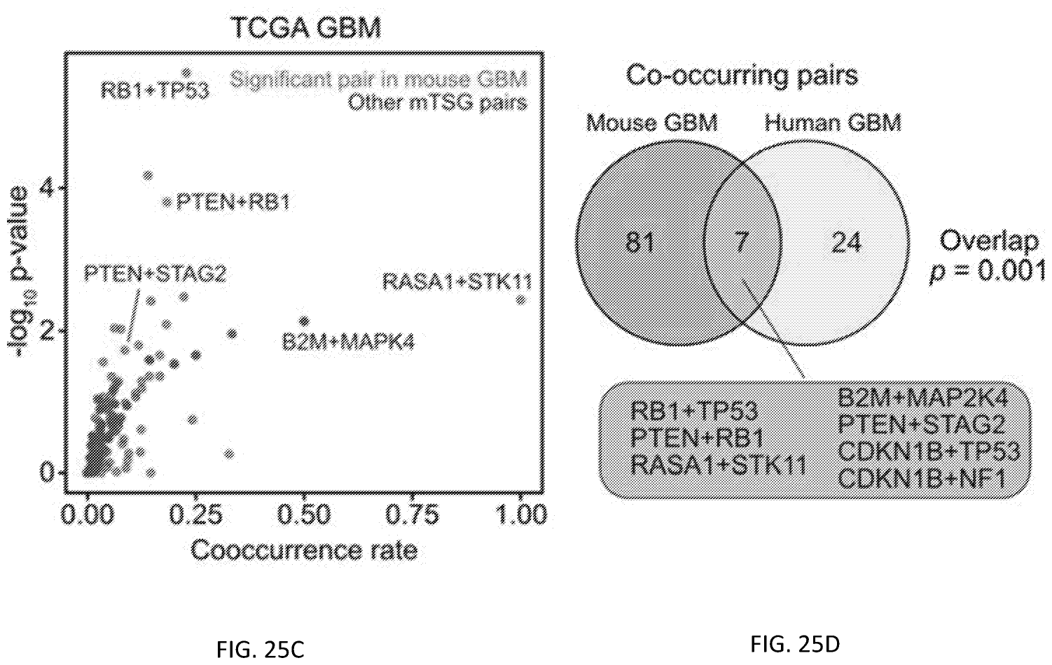

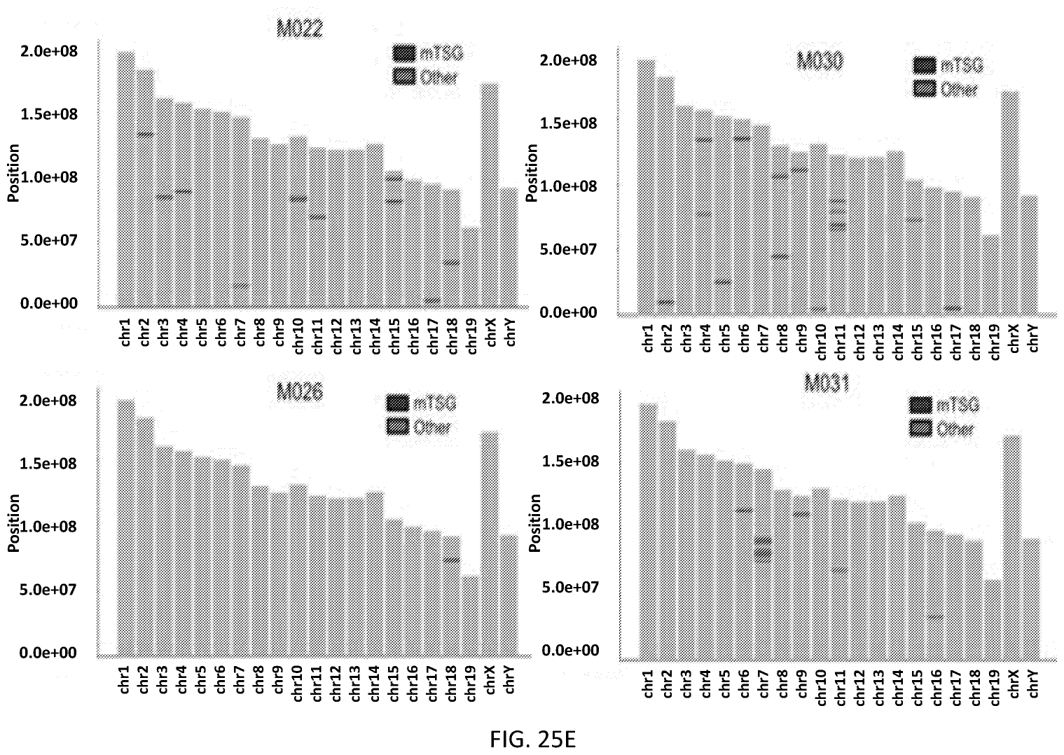

To generate an unbiased map of co-drivers, the co-occurrence rate of double-mutations for each gene pair we calculated (FIGS. 24A-24B). This analysis showed that 76 gene pairs out of a total of 1540 possible pairs were statistically significant in terms of co-occurrence (hypergeometric test, FDR adjusted q<0.05). The Nf1+Pten pair emerged as the top pair (co-occurrence rate= 18/21=85.7%, hypergeometric test, p=7.53*10⁻⁸) (FIGS. 24A-24C). Interestingly, several previously undocumented combinations emerged, such as Kdm5c+Gata3 (co-occurrence rate=77.8%, hypergeometric test, p=6.04*10⁻⁶), and B2m+Pik3r1 (70.0%, p=2.28*10⁻⁵) (FIGS. 24A-24C). In addition, correlation analysis of summed mutant frequencies for each pair of genes was performed across all mice (FIGS. 24D-24E). 22.9% ( 352/1540) of the gene pairs were positively correlated (Spearman correlation>0, FDR adjusted q<0.05) (FIGS. 24D-24E). The most significantly correlated gene pair was again Nf1+Pten (Spearman correlation=0.861, p=3.34*10⁻⁸) (FIGS. 24D-24F), along with other representative pairs such as Cdkn2a+Ctcf (correlation=0.792, p=2.41*10⁻⁶) (FIG. 24G), B2m+Notch1 (correlation=0.789, p=2.82*10⁻⁶), and Apc+Pik3r1 (correlation=0.774, p=5.77*10⁻⁶) (FIGS. 24D-24E). Exclusion of Trp53 revealed largely identical results for the remaining genes (FIGS. 25A-25B). Of note, a subset of the significantly co-occurring pairs were also found to be co-mutated in human GBM, including the pairs of RB1+TP53, PTEN+RB1, RASA1+STK11, B2M+MAP2K4, PTEN+STAG2, CDKN1B+TP53 and CDKN1B+NF1 (FIGS. 25C-25D). These data revealed co-occurrence and correlation relationships of specific mutations during glioblastoma progression in vivo.

### Example 4: Minipool Validation of Individual Drivers and Combinations

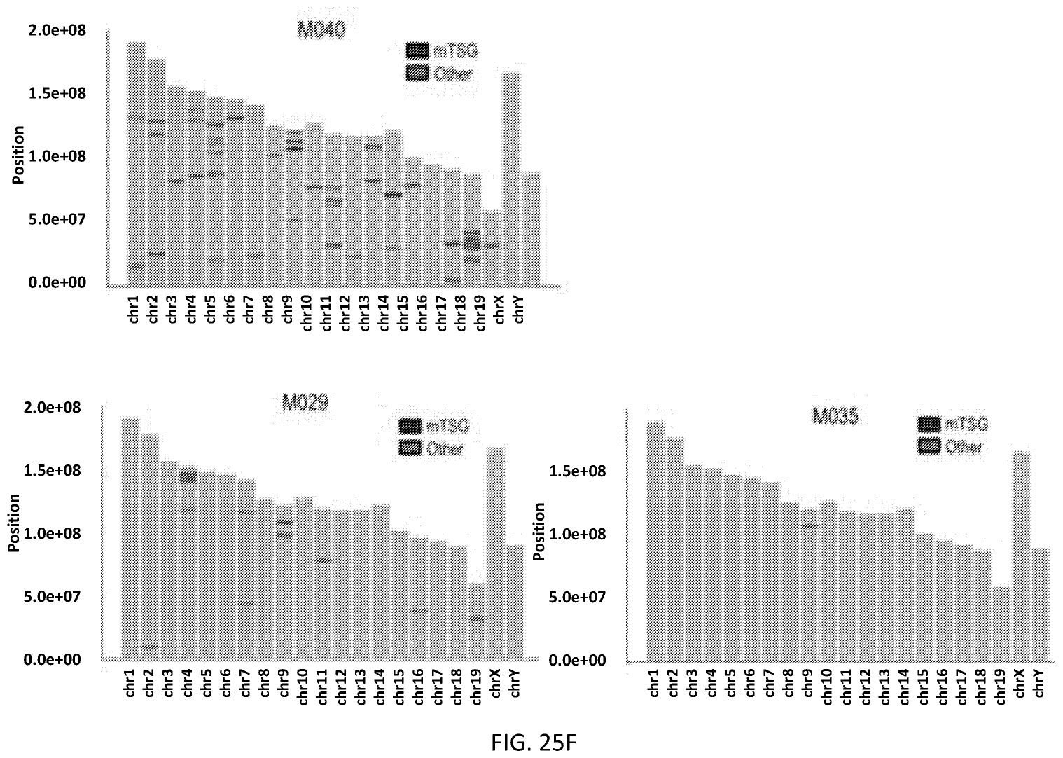

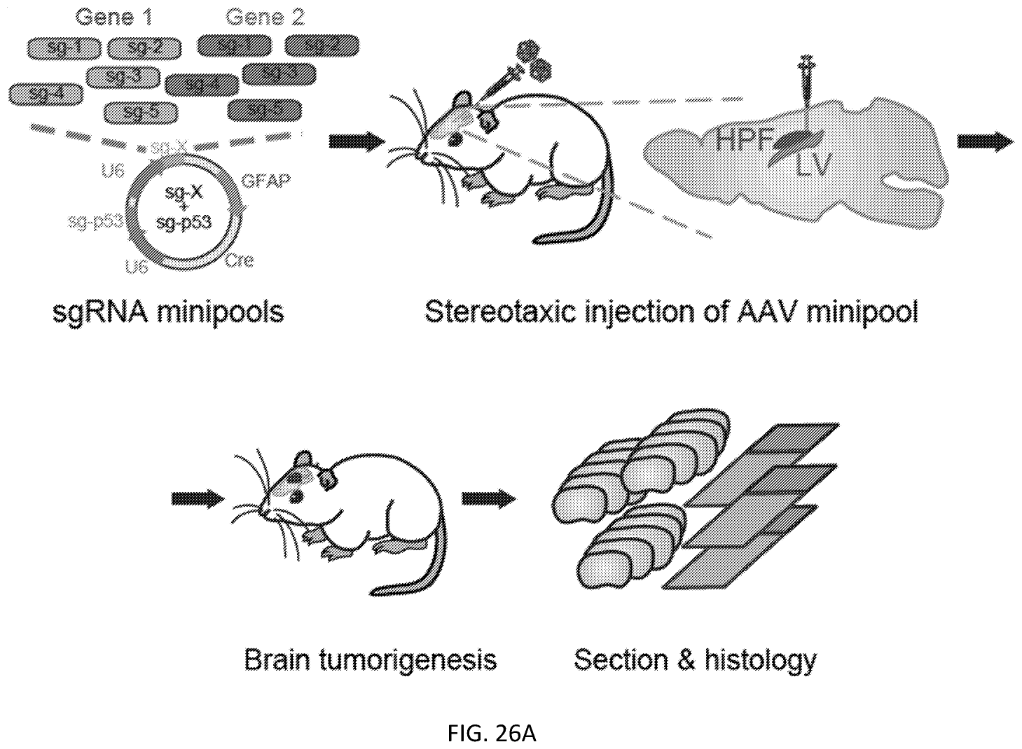

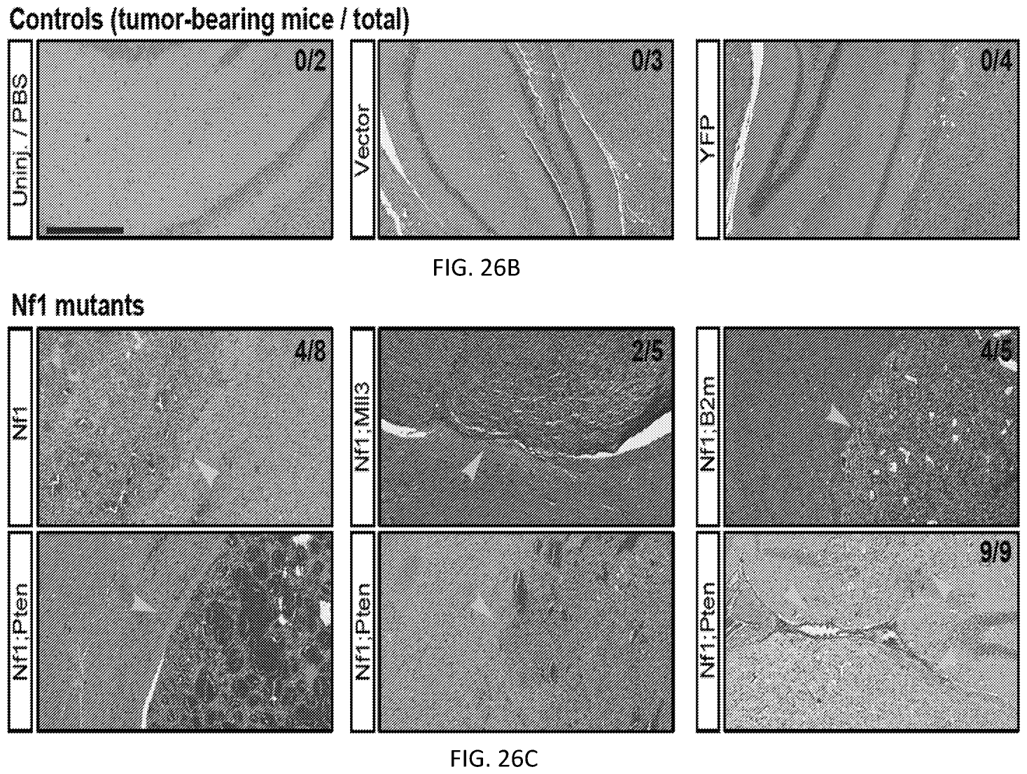

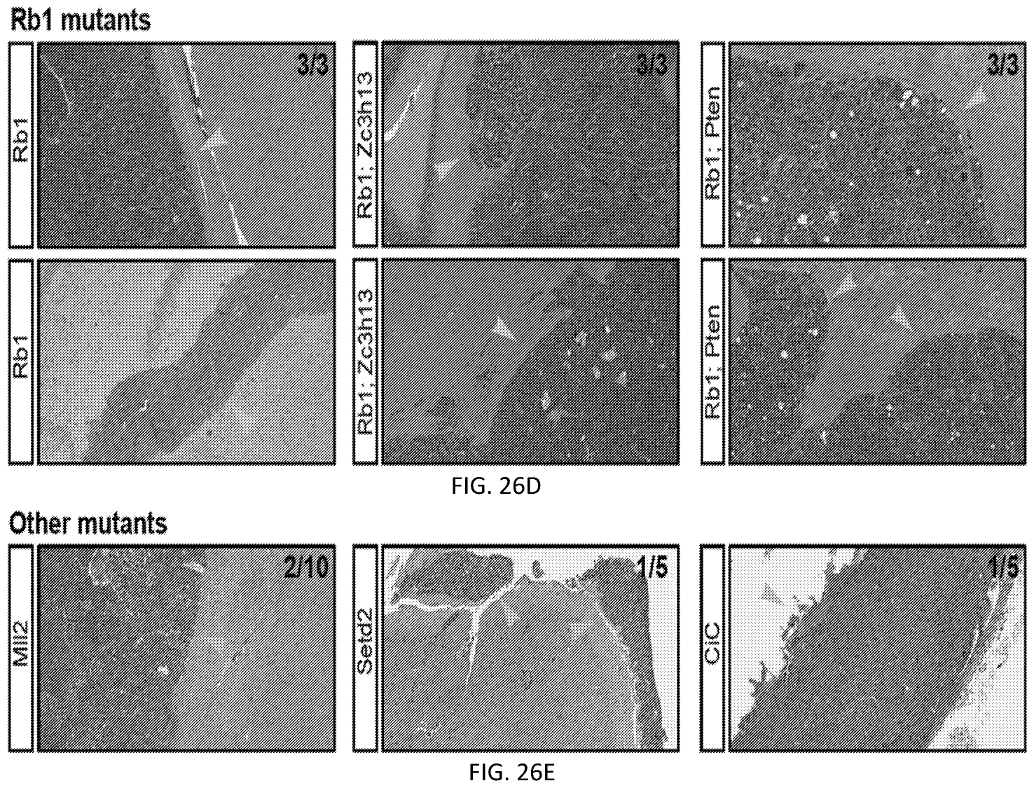

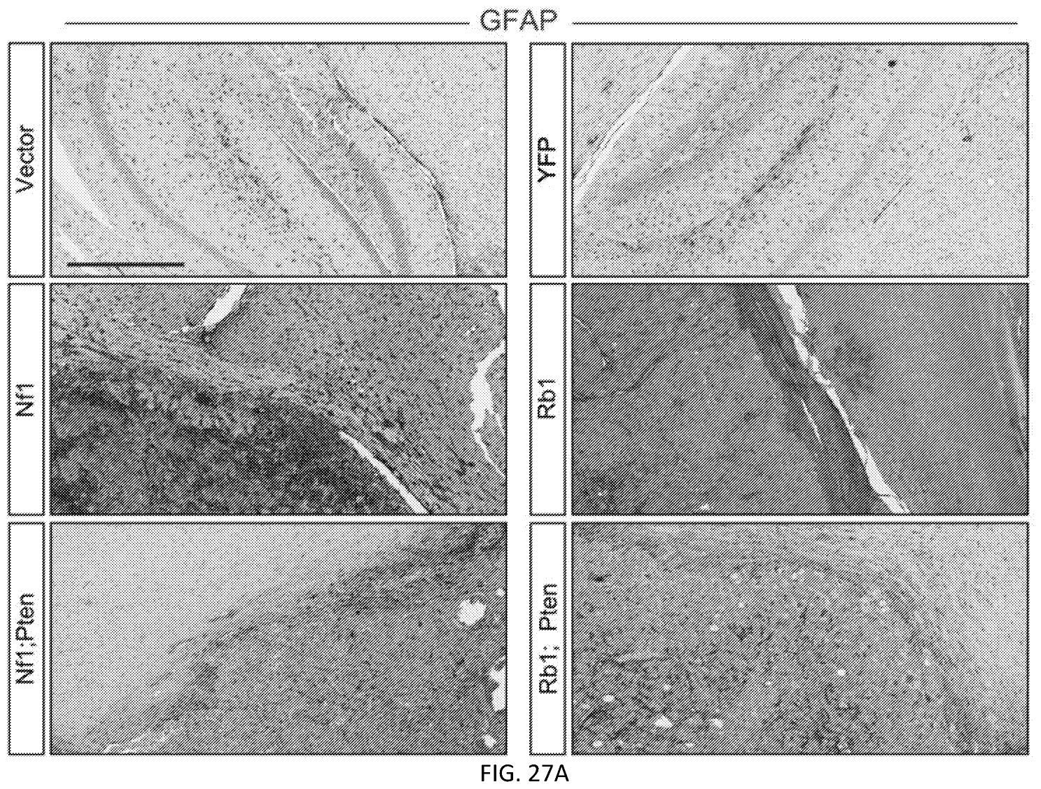

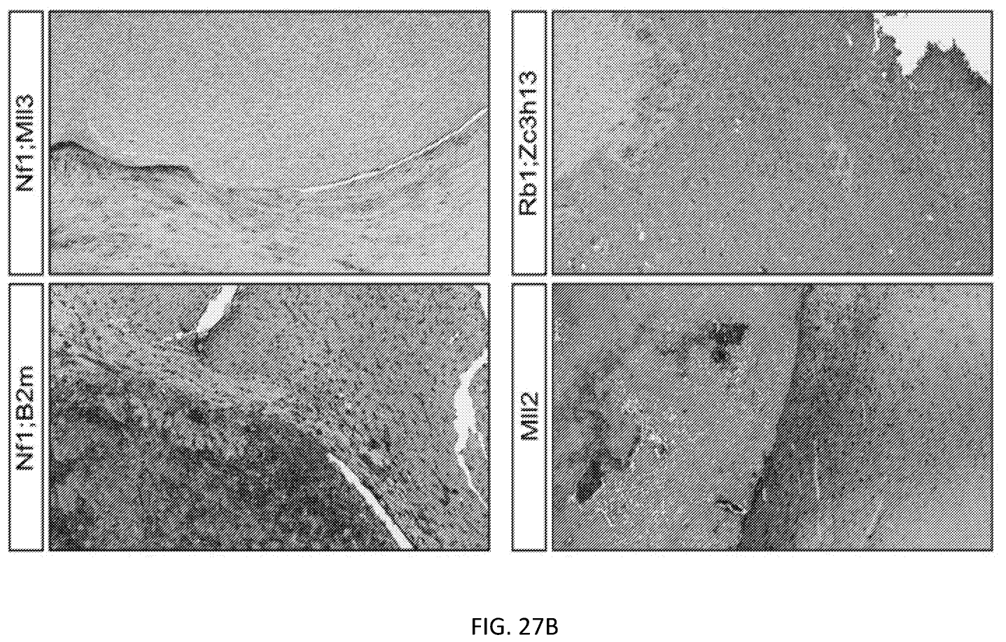

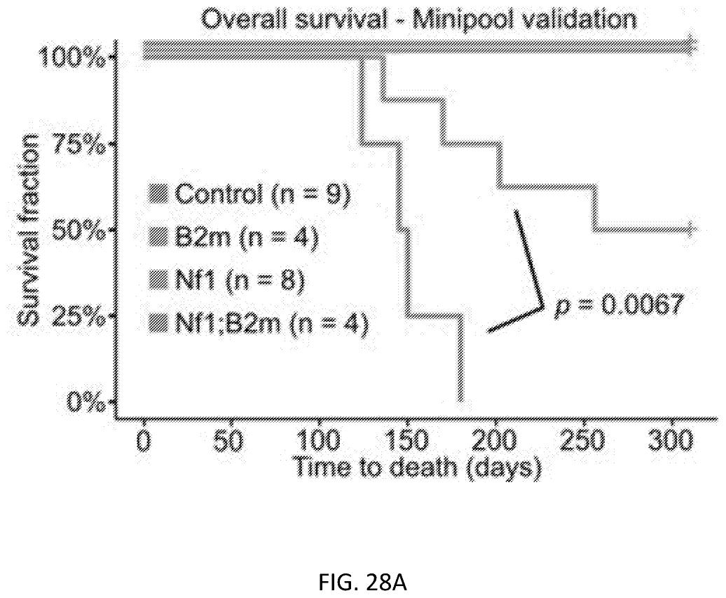

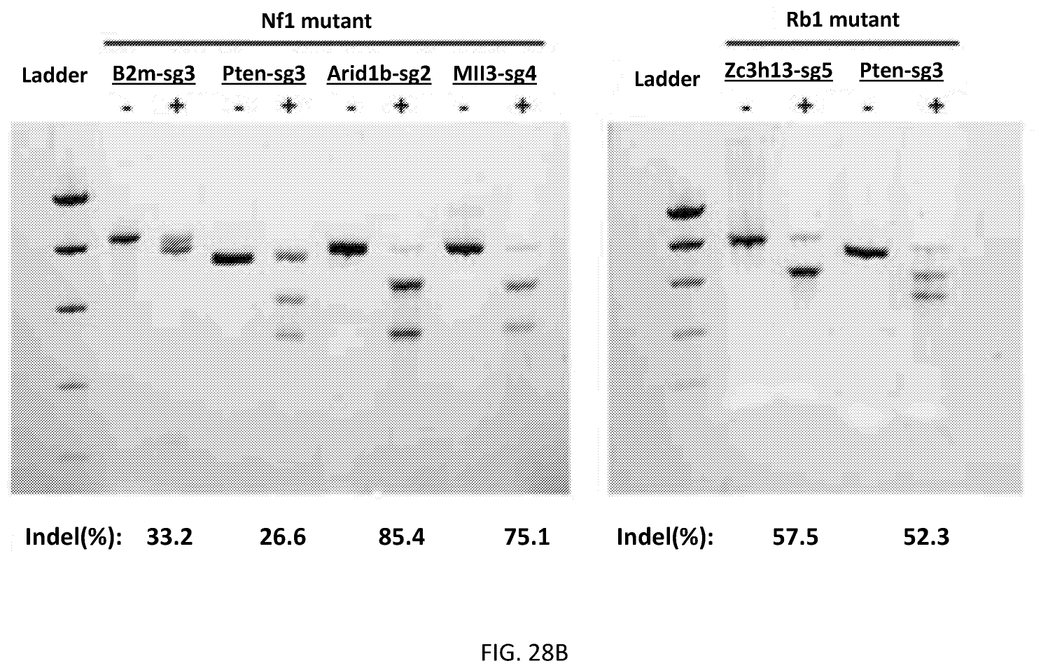

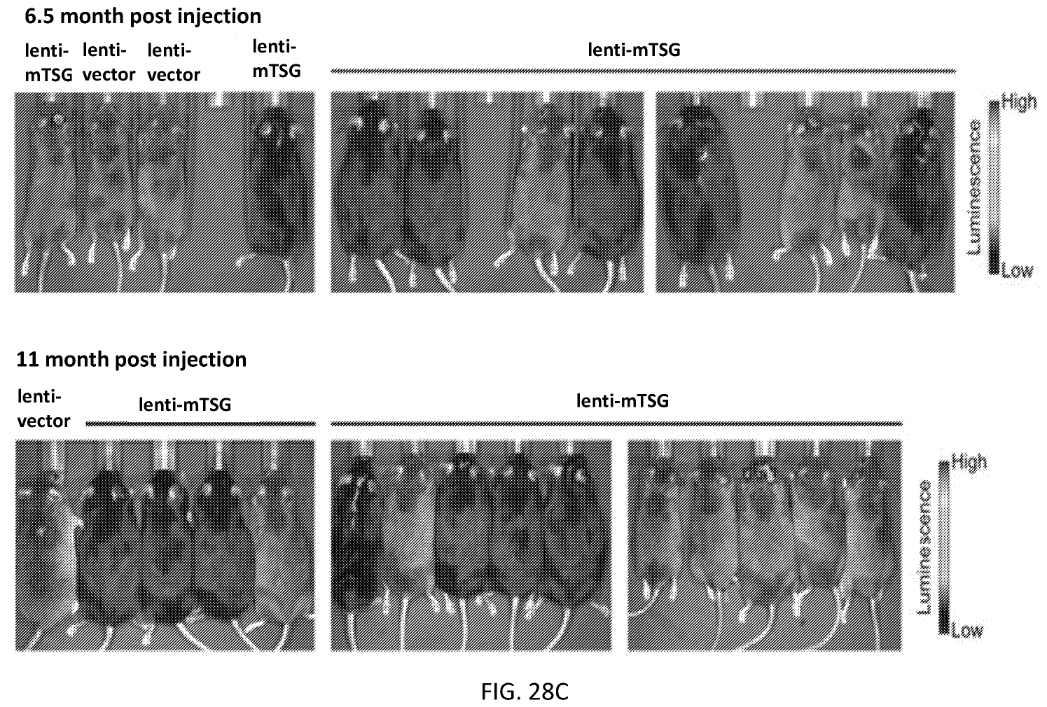

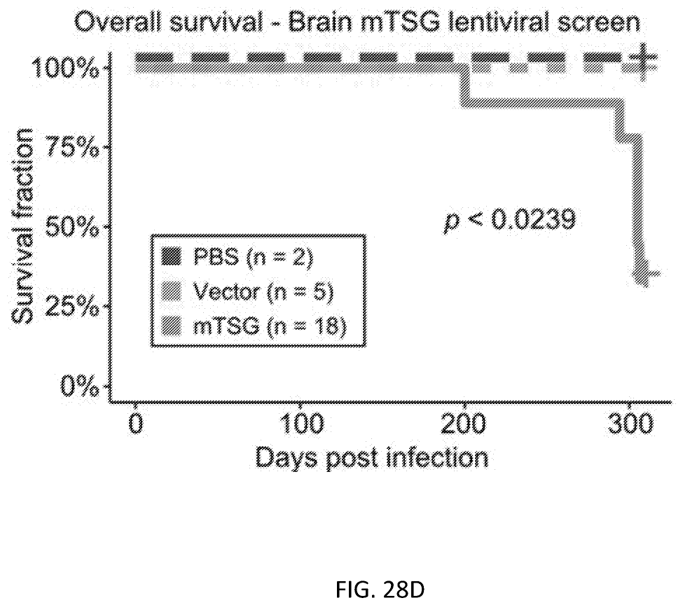

Several of the highly represented individual drivers or combinations were tested using an sgRNA minipool validation approach (FIG. 26A). All of the un-injected (n=2), EYFP (n=4) and empty vector (n=3) injected mice survived and maintained good body condition for the whole duration of the study. Control mice sacrificed from 4 to 11 months after treatment were devoid of any observable tumors by histology analysis (FIG. 26B, FIGS. 27A-27B), again indicating that without mutagenesis, or with Trp53 disruption alone, LSL-Cas9 mice did not develop brain tumors. In contrast, within 11 months post injection, 50% ( 4/8) of mice receiving AAVs containing Nf1 sgRNA minipool developed macrocephaly, poor body condition score and tumors (compared to all 9 control mice, two-tailed Fisher's exact test, p=0.029). All mice receiving Nf1; Pten ( 9/9, 100%, p=4.11*10⁻⁵) and Nf1; B2m minipools ( 4/4, 100%, p=0.0014) developed macrocephaly, poor body condition score and large tumors (FIG. 26C). Notably, mice receiving Nf1; B2m minipools had significantly worse survival than mice receiving Nf1 minipools alone (p=0.0067) (FIG. 28A), implying that loss of antigen presentation in cancer cells likely accelerates GBM progression in immunocompetent mice. All mice receiving Rb1, Rb1; Pten, or Rb1; Zc3h13 minipools ( 3/3, 100%, p=0.0045 for all three groups) developed macrocephaly, poor body condition score and large tumors (FIG. 26D). For the same duration of study (maximum 11 months), smaller fractions of mice receiving the AAV sgRNA minipools targeting Arid1b; Nf1 ( 4/9), Mll3; Nf1 (⅖), Mll2 ( 2/10), Cic (⅕), Cic; Pten (¼), Setd2 (⅕), and Gata3,Mll3 (⅕) developed tumors. Collectively, half ( 40/80, or 50%) of the mice receiving AAV sgRNA minipools targeting any of the single genes or gene pairs developed brain tumors within 11 months (collective validation vs. all controls, two-tailed Fisher's exact test, p=0.004). These data indicated that mutating these individual genes or combinations in combination with Trp53 causes GBM in fully immunocompetent animals.

Interestingly, brain tumors with Nf1 mutations displayed highly polymorphic pathological features, with diverse fibroblastic cell morphologies, regions of necrosis and large hemorrhages (FIG. 26C), yet were almost always GFAP-positive (FIG. 27A-27B). In sharp contrast, tumors with Rb1 mutations were composed of round cells with dense nuclei, frequently with proliferative spindles and giant cells with massive nuclear aneuploidy and pleiomorphism, but rarely with regions of necrosis or large-area hemorrhage (FIG. 26D), and often contained mixtures of GFAP-positive and GFAP-negative cells (FIG. 27A-27B).

### Example 5: Transcriptomic Characterization of Tumors with Differing Mutational Backgrounds

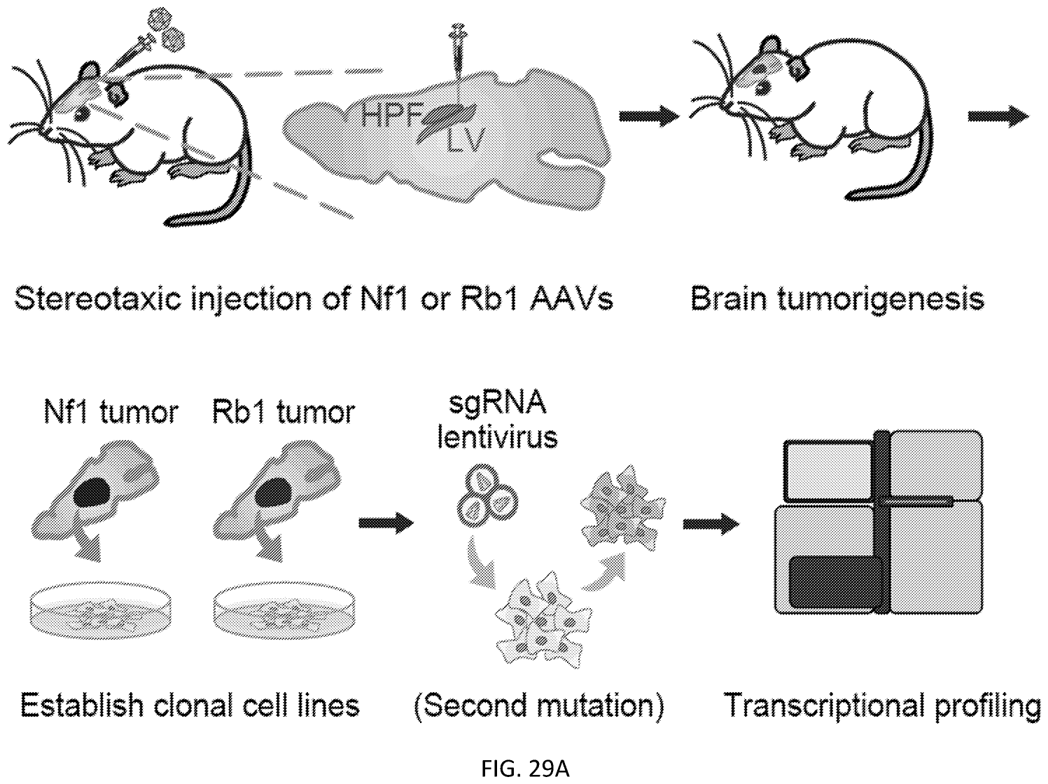

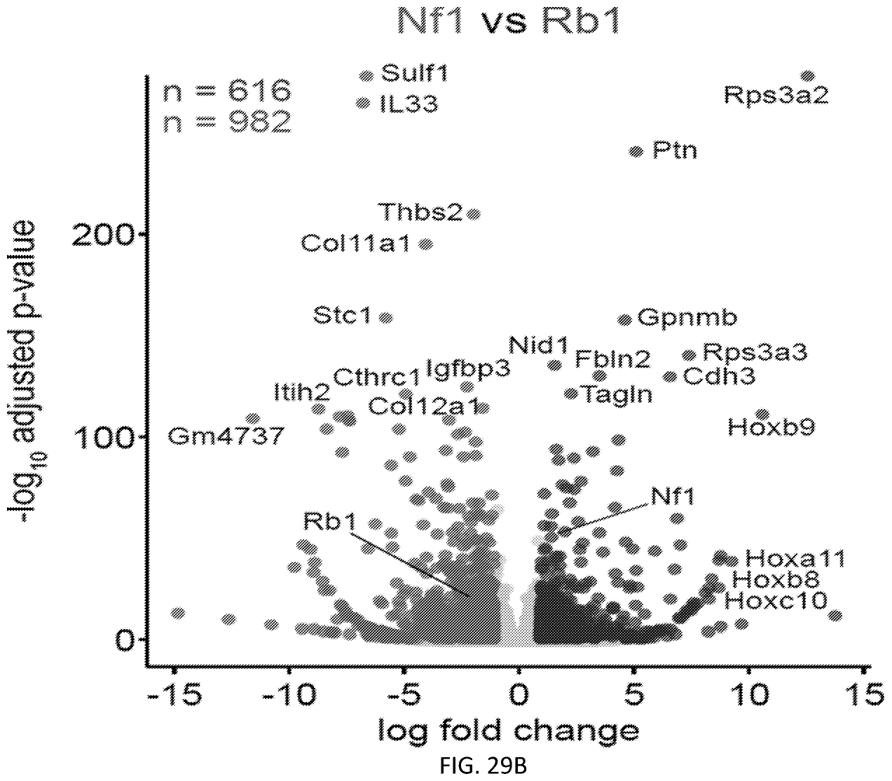

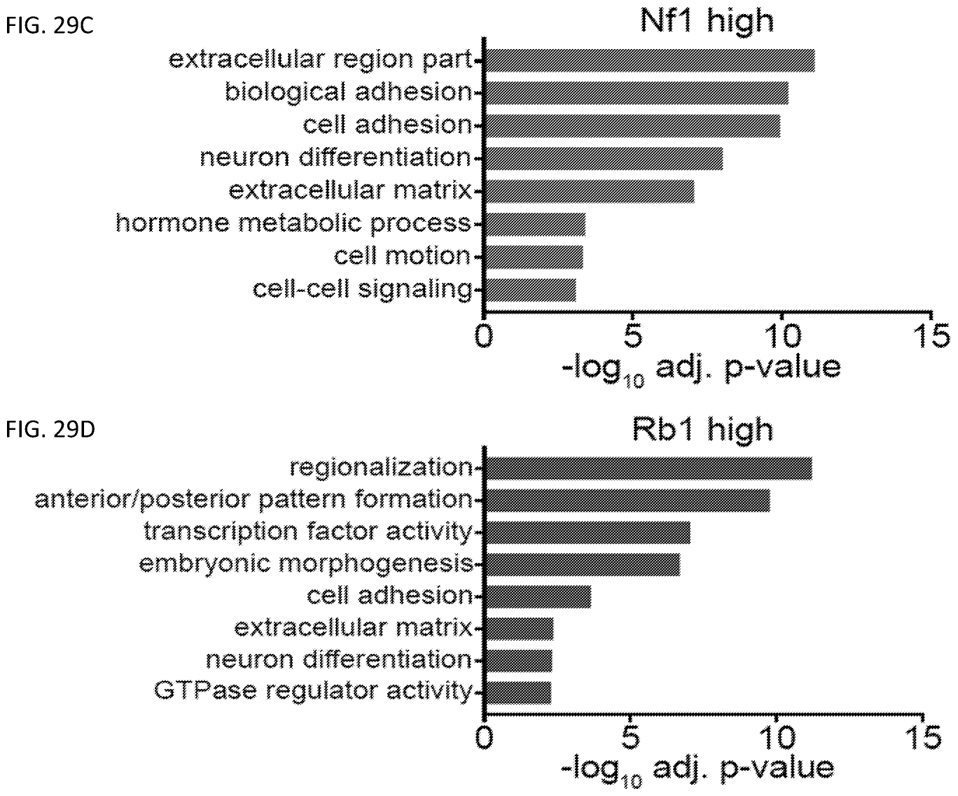

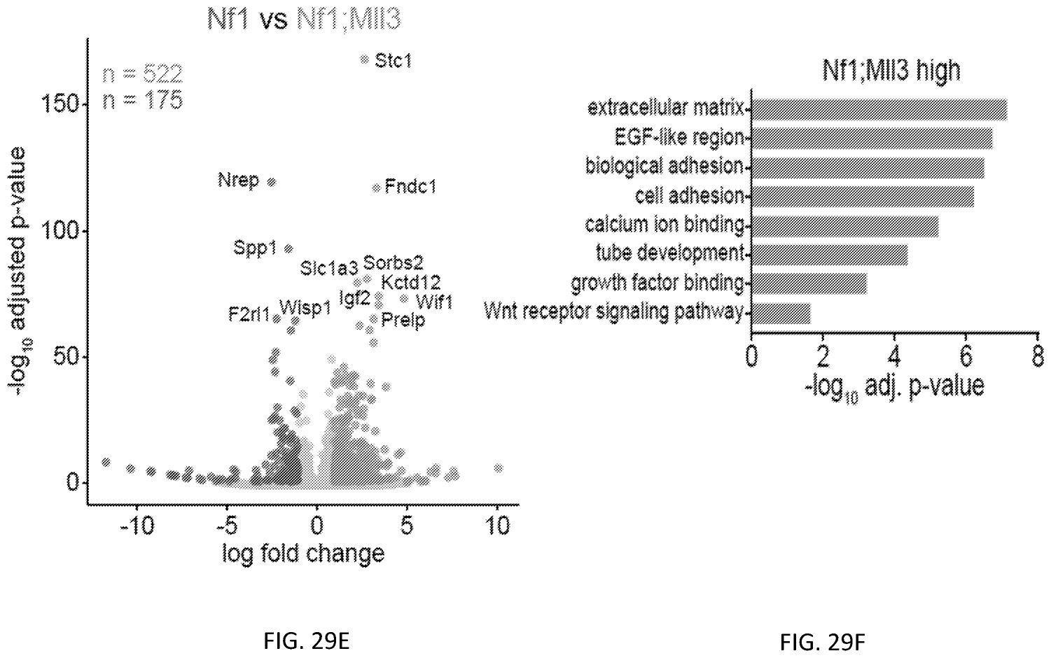

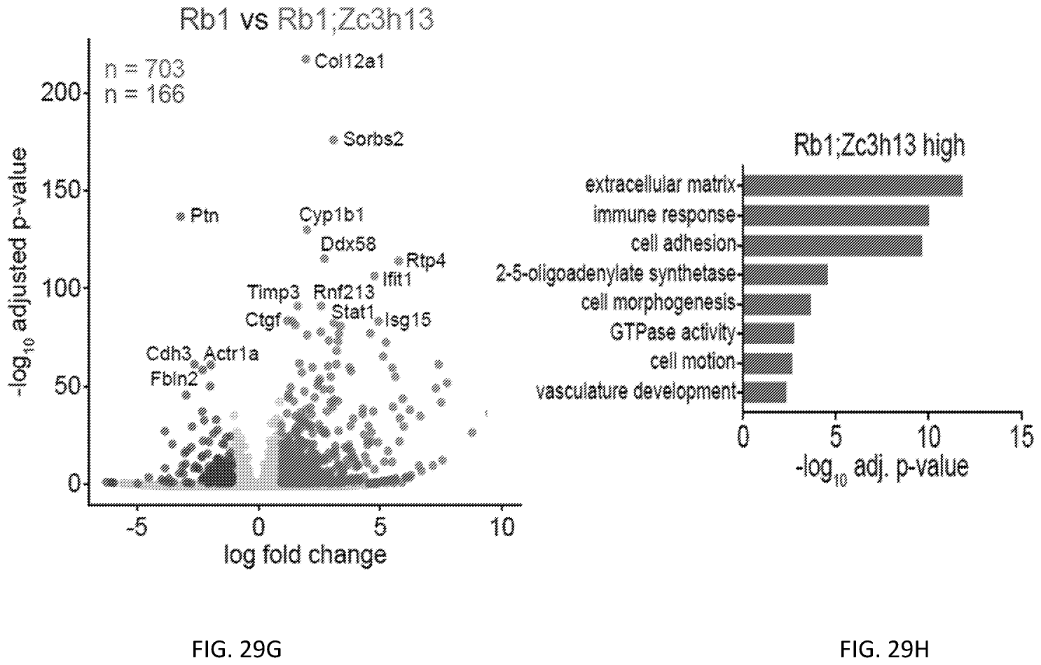

The molecular underpinnings of gliomagenesis driven by different combinations of drivers was investigated (FIG. 29A; FIG. 28B). mRNA-seq was performed to profile the transcriptome of these mutant glioma cells (Nf1, Nf1; ; Mll3, Rb1, and Rb1; Zc3h13, n=3 cell replicates each). Comparing Nf1-mutant and Rb1-mutant cells, 616 genes were more highly expressed in Rb1 cells (Benjamini-Hochberg adjusted p<0.05 and log fold change>1), while 982 genes were more highly expressed in Nf1 cells (FIG. 29B). Gene ontology analysis of the genes associated with higher expression in Nf1-mutant cells revealed multiple enriched categories (Benjamini-Hochberg adjusted p<0.05), including extracellular region part, biological adhesion, neuron differentiation, hormone metabolic process, cell motion, and cell-cell signaling (FIG. 29C). Gene ontology analysis of the genes associated with higher expression in Rb1-mutant cells revealed a distinct set of enriched categories (adjusted p<0.05), which surprisingly included regionalization, anterior/posterior pattern formation, transcription factor activity, embryonic morphogenesis, cell adhesion, extracellular matrix, neuron differentiation, and GTPase regulator activity (FIG. 29D). Strikingly, a total of 13 Homeobox genes were among the top-40 upregulated genes in Rb1 mutants.

To understand the direct effect of additional mutations on the transcriptome of these cells, Nf1; ; Mll3 were compared to Nf1 cells, and Rb1; Zc3h13 were compared to Rb1 cells. 522 genes were upregulated in Nf1; Mll3 compared to Nf1 cells, while 175 were downregulated (FIG. 29E). Gene ontology analysis of the upregulated genes in Nf1; Mll3 cells revealed enrichment of extracellular matrix, EGF-like region, biological adhesion, calcium ion binding, tube development, and growth factor binding (FIG. 29F). Comparing Rb1; Zc3h13 to Rb1 cells revealed 703 upregulated and 166 downregulated genes (FIG. 29G). Rb1; Zc3h13-high genes were enriched in categories such as extracellular matrix, immune response, cell adhesion, 2-5-oligoadenylate synthetase, cell morphogenesis, GTPase activity, cell motion, and vasculature development (FIG. 29H). Collectively, these findings indicate that the addition of an Mll3 mutation significantly alters the transcriptome of Nf1 mutant cells, as does the addition of a Zc3h13 mutation on Rb1 mutant cells.

### Example 6: Secondary Mutations Influence the Transcriptome and Engender Chemotherapeutic Resistance

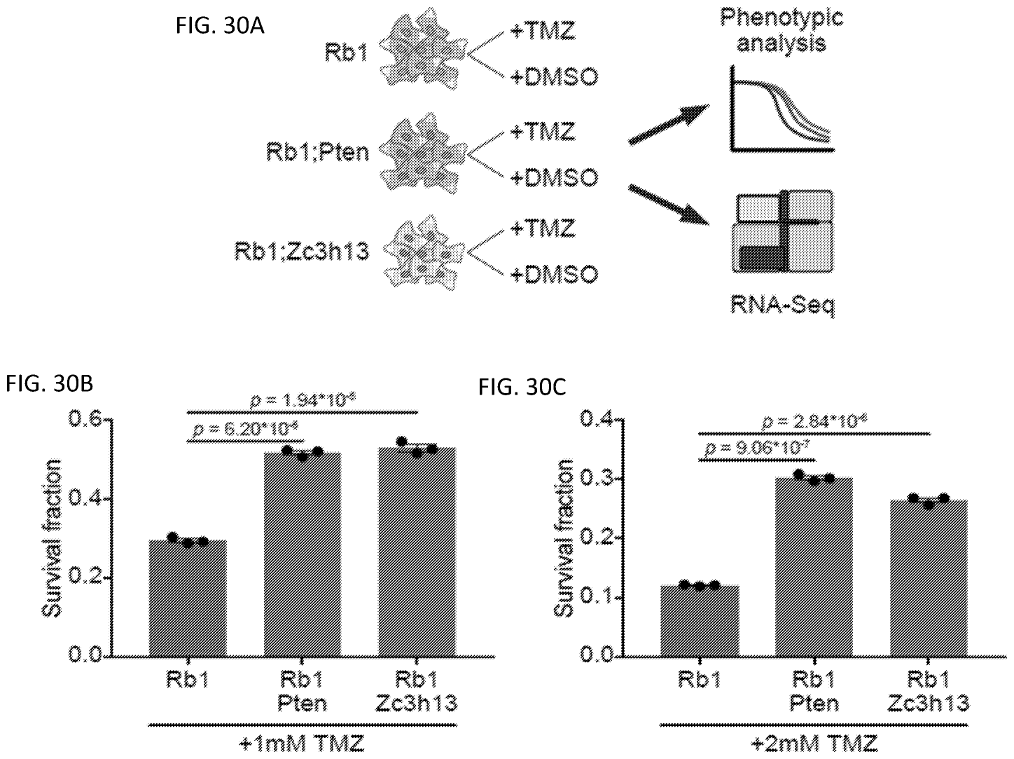

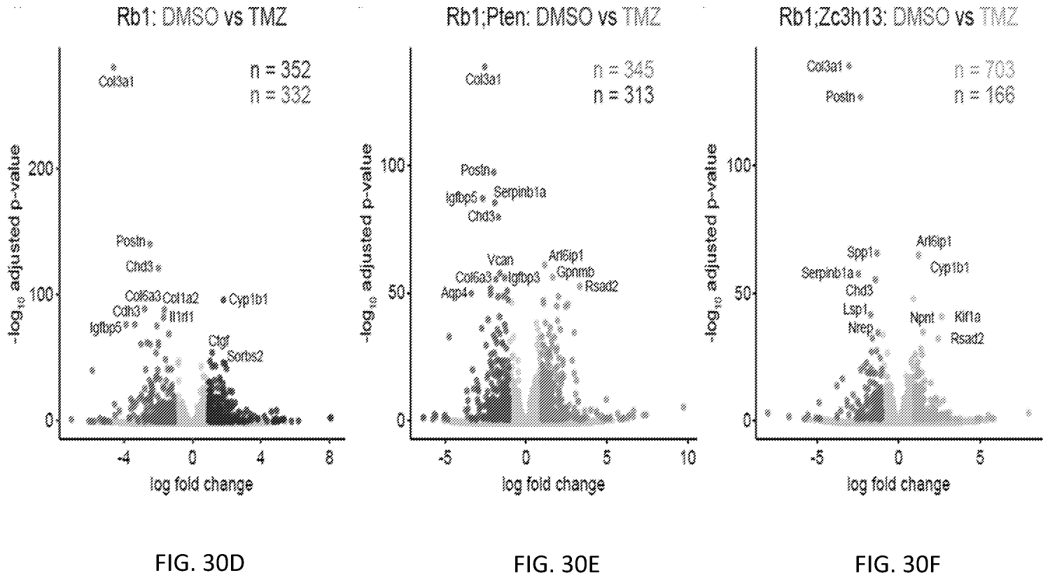

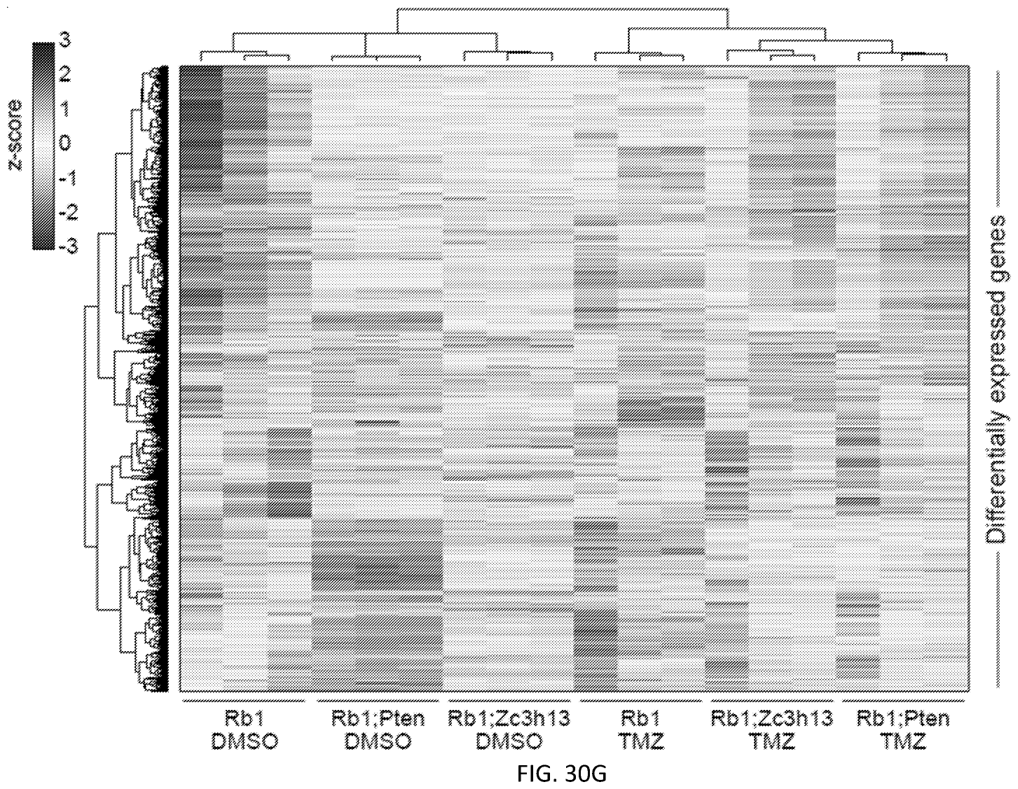

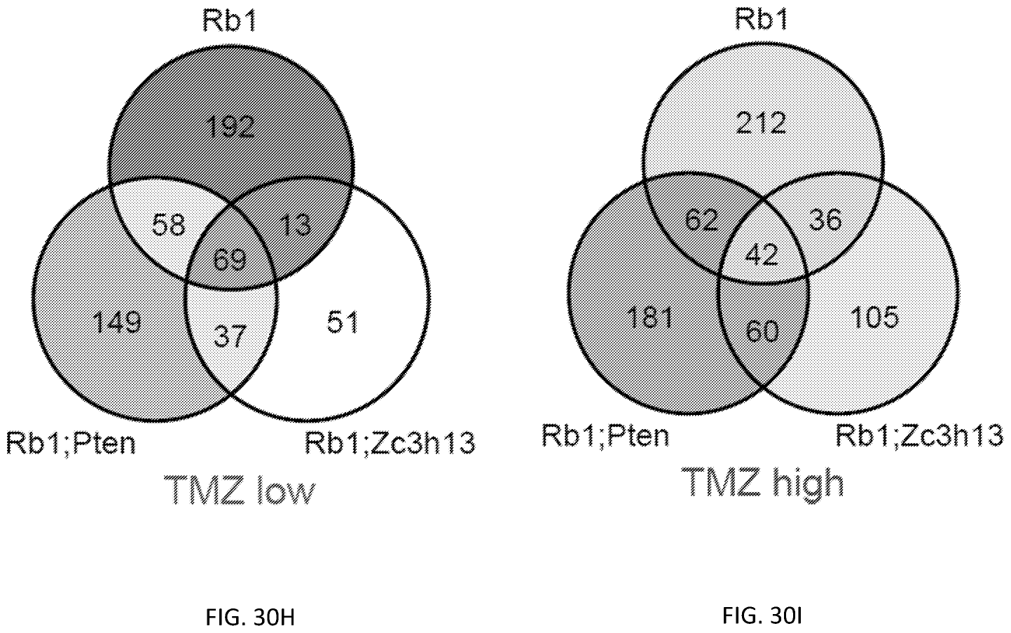

As GBM remains a challenging cancer type to treat, understanding the molecular changes underlying drug response is of profound importance. Thus, drug-treatment-RNA-seq experiments were performed to investigate the transcriptome responses of AAV-CRISPR-induced GBM cells (Rb1, Rb1; Pten and Rb1; Zc3h13) to TMZ, a chemotherapeutic with significant albeit small survival benefit for GBM patients, among the only 4 currently approved drugs for this disease (FIG. 30A). Drug response phenotyping showed that Zc3h13 loss-of-function rendered Rb1 cells significantly more resistant to 1 mM TMZ, similar to Pten loss-of-function (two-tailed t-test, t₄=31.32 and t₄=23.51, p=6.20*10⁻⁶ and p=1.94*10⁻⁵, for Rb1; Pten vs. Rb1 and Rb1; Zc3h13 vs. Rb1, respectively) (FIG. 30B). These differences were also observed with 2 mM TMZ (t₄=50.69 and t₄=38.10, p=9.06*10⁻⁷ and p=2.84*10⁻⁶, for Rb1; Pten vs. Rb1 and Rb1; Zc3h13 vs. Rb1, respectively) (FIG. 30C). Given the differential responses among these three genotypes, we performed mRNA-seq to profile the transcriptome of these mutant cells treated with TMZ as compared to DMSO controls. Differential expression analyses of TMZ and DMSO treated cells from each of the three genotypes revealed systematic changes in gene expression (FIG. 30D-30F). Collectively, the differentially expressed genes in the TMZ vs. DMSO comparisons uncovered a molecular map of the transcriptomic differences between genotypes in response to TMZ treatment (FIG. 30G).

Of the genes that were significantly reduced upon TMZ treatment in each group, a total of 69 genes were shared among all three genotypes (FIG. 30H), indicating that these genes are a common transcriptional response to TMZ. As Rb1; Zc3h13 and Rb1; Pten cells exhibited greater survival fractions with TMZ treatment when compared to Rb1 cells, we identified 37 genes that were significantly reduced upon TMZ treatment in Rb1; Zc3h13 and Rb1; Pten cells, but not in Rb1 cells (FIG. 30H). As for the genes that were significantly induced by TMZ, a total of 42 genes were common in all three genotypes (FIG. 30I), representing a shared TMZ-induced gene signature Interestingly, we identified 60 genes that were upregulated upon TMZ treatment in Rb1; Zc3h13 and Rb1; Pten cells, but not in Rb1 cells. These included Arl6ip1, which encodes a protein that has been shown to suppress cisplatin-induced apoptosis in cancer cells, and Cd274 (also known as PD-L1), which encodes the ligand for the inhibitory receptor PD-1 that is currently a major focus of investigation in cancer immunotherapy. Taken together, the transcriptomic analyses provide unbiased molecular signatures underlying the increased TMZ-resistance upon Zc3h13 or Pten mutations in Rb1-oncotype glioma cells, suggesting that the precise combinations of mutational drivers present in individual GBMs directly influence therapeutic responses.

### Example 7: Discussion

GBM is one of the most lethal cancer types, and is the major malignancy in the human brain. Currently, identifying mutations that are cancer drivers in patients is not straightforward or necessarily accurate, as they are often inferred from mutation type or frequency. Determining whether these alterations are bona fide driver mutations requires direct functional testing, but such testing is generally performed one gene at a time in cell lines or mouse models. Animal models of cancer in the past several decades have significantly advanced our understanding of the roles of oncogenes, tumor suppressor genes and other modulating factors in cancer progression and therapeutic responses. Development and applications of mouse models of GBM have led to profound progress in our understanding of tumor initiation, stem cell populations, progression and therapeutic responses of GBM. However, animal studies to date are limited to small numbers of genes. Direct in vivo high-throughput mutational analysis of functional cancer drivers in the mouse brain has been difficult, due to the nature of biological complexity in vivo. It is challenging to perform high-throughput analysis of mutants in autochthonous models of cancer; that is, models in which the tumors directly evolve from normal cells at the organ site in situ in immunocompetent mice and without cellular transplantation.

This challenge has been overcome herein by developing a focused AAV-CRISPR library with AAV encoding functional elements and sgRNA pools targeting a top pan-cancer putative TSG set, in combination with the conditional LSL-Cas9 transgenic mice. Herein, a powerful platform was developed to perform high efficiency AAV-CRISPR mediated pooled mutagenesis for direct in vivo analysis of many genes in mice. AAV usually does not integrate into the genome, except under certain circumstances (low rate of integration at AAVS1 locus). Thus, AAV-encoded transgenes such as exogenously supplied sgRNAs do not replicate as cells divide during tumor progression, limiting the direct readout of mTSG library by PCR amplification of the sgRNA cassette itself. Successful readout of driver mutations in the targeted genes was achieved by directly sequencing the predicted sgRNA cutting sites for indels using targeted captured sequencing at high coverage. The approach of AAV-CRISPR mediated pooled mutagenesis in conjunction with targeted capture sequencing provides an efficient platform for massively parallel analysis of GBM drivers directly in vivo. The AAV-CRISPR genetically engineered mouse tumor models described herein were developed in fully immunocompetent mice, preserve the native tumor microenvironment, and therefore can be used in high-throughput screening of immunotherapy responses in vivo.

With this platform, high-throughput gene editing was demonstrated in multiplexed autochthonous GBM models in fully immunocompetent mice. This study provides a dynamic picture of tumor suppressors in vivo, revealing the relative selective strength of mutations in these genes when competing together in the astrocytes in the brain, as well as the oncotypic variations between individuals. Moreover, co-occurrence analysis revealed strongly enriched combinations of mSMGs that far exceeded what would be expected by chance. These include well-documented combinations (e.g. Nf1-Pten), indicating technological robustness and disease relevance, as well as previously unexpected functional combinations (e.g. Gata3-Kdm5c, B2m-Pik3r1) with unknown molecular mechanisms for the selection forces that evidently favor them.

Across all genes tested, the mutation frequencies in this highly complex mouse model of GBM significantly correlate with the mutation frequencies in human patients from two large independent cohorts (TCGA and Yale Glioma bank), suggesting the clinical relevance of the findings. Several of the novel SMGs highly enriched in this mouse study have also been associated with GBM in the clinical setting, such as B2M, CIC, MLL2, MLL3, SETD2, ZC3H13 and ARID1B. Because differences in driver mutations can dramatically affect treatment efficacies in pre-clinical animal models and in patients, a functional understanding of cancer drivers is therefore essential for precision medicine.

A lentiCRISPR direct in vivo screen was also performed in GBM, using the same mTSG library (FIGS. 28C-28D). The AAV-mTSG CRISPR library resulted in more robust gliomagenesis in vivo compared to lentiCRISPR mTSG, in terms of latency (death of first animal, 84 vs. 200 days), survival (median survival, 4 vs. 10 months), and penetrance (100% vs. 67%). However, AAVs usually do not integrate into the genome, except under certain circumstances (low rate of integration at AAVS1 locus). Thus, AAV-encoded transgenes such as exogenously supplied sgRNAs do not replicate as cells divide during tumor progression, limiting the readout of the mTSG library by PCR amplification of the sgRNA cassette itself. Instead, successful readout of driver mutations was achieved by sequencing the predicted sgRNA cutting sites using ultra-deep targeted captured sequencing. A key advantage of this approach is the ability to perform high-throughput mutagenesis in an autochthonous model of GBM, in which tumors directly evolve from normal cells at the organ site in situ in immunocompetent mice, without cellular transplantation. This platform can be readily extended to study other types of cancer for tumor progression, as well as therapeutic responses in vivo.

Taken altogether, this study provides a systematic and unbiased molecular landscape of functional tumor suppressors in an autochthonous mouse model of GBM, opening the path for high-throughput analysis of cancer gene phenotypes directly in vivo.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of determining treatment for a subject suffering from glioblastoma, the method comprising:
contacting a plurality of Adeno-Associated Virus- Clustered Regularly Interspaced Short Palidromic Repeats (AAV-CRISPR) vectors with a sample from the subject, wherein the vectors comprise Cas9 and a plurality of nucleotide sequences homologous to a plurality of tumor suppressor genes (TSGs), thus generating a reaction mixture;
sequencing a plurality of nucleic acids isolated from the reaction mixture; and
analyzing the data from the sequencing as to identify any mutation in the plurality of nucleic acids,
whereby treatment for the subject suffering from glioblastoma is determined based on presence and/or nature of any mutation in the plurality of nucleic acids.

2. A method of determining at least one glioblastoma driver mutation in a sample, the method comprising:
contacting a plurality of AAV-CRISPR vectors with the sample, wherein the vectors comprise Cas9 and a plurality of nucleotide sequences homologous to a plurality of tumor suppressor genes (TSGs), thus generating a reaction mixture;
sequencing a plurality of nucleic acids isolated from the reaction mixture; and
analyzing the sequencing data as to identify any glioblastoma driver mutation therein.

3. The method of claim 1, wherein the plurality of nucleotide sequences homologous to a plurality of TSGs comprises at least one selected from the group consisting of SEQ ID NOs. 1-280.

4. The method of claim 1, wherein the plurality of nucleotide sequences homologous to a plurality of TSGs comprises SEQ ID NOs. 1-280.

5. The method of claim 1, wherein the sequencing comprises targeted capture sequencing.

6. The method of claim 1, wherein the mutation comprises a nucleotide insertion.

7. The method of claim 6, wherein the insertion comprises more than one nucleotide base.

8. The method of claim 1, wherein the mutation comprises a nucleotide deletion.

9. The method of claim 8, wherein the deletion comprises more than one nucleotide base.

10. The method of claim 1, wherein the sample comprises a plurality of glioma cells from the subject.

11. The method of claim 1, wherein the sample comprises a tumor from the subject.

12. The method of claim 1, further comprising monitoring cell proliferation in the reaction mixture.

13. An AAV-CRISPR library comprising a plurality of AAV vectors comprising Cas9 and a plurality of nucleic acids homologous to a plurality of Tumor Suppressor Gene (TSGs).

14. The AAV-CRISPR library of claim 13, wherein the plurality of nucleic acids comprises at least one selected from the group consisting of SEQ ID NOs. 1-280.

15. The AAV-CRISPR library of claim 13, wherein the plurality of nucleic acids comprises SEQ ID NOs. 1-280.

16. A kit for determining at least one driver mutation in a glioblastoma sample comprising:
an AAV-CRISPR library comprising a plurality of AAV vectors comprising Cas9 and a plurality of nucleic acids homologous to a plurality of Tumor Suppressor Gene (TSGs),
reagents for measuring the at least one driver mutation, and
instructional material for use thereof.

17. The kit of claim 16, wherein the plurality of nucleic acids comprises at least one selected from the group consisting of SEQ ID NOs. 1-280.

18. The kit of claim 16, wherein the plurality of nucleic acids comprises SEQ ID NOs. 1-280.

19. A method of determining at least one glioblastoma driver mutation in vivo in a glioblastoma-affected subject, the method comprising:
administering into the brain of the subject a plurality of AAV-CRISPR vectors, wherein the AAV-CRISPR vectors comprise Cas9 and a plurality of short guide RNAs (sgRNAs) homologous to a plurality of tumor suppressor genes (TSGs); and
sequencing a plurality of nucleic acids isolated from the subject's glioblastoma; whereby analysis of the sequencing data indicates whether any glioblastoma driver mutation is present in the subject's glioblastoma.

20. The method of claim 19, wherein the plurality of sgRNAs comprises at least one selected from the group consisting of SEQ ID NOs. 1-280.

21. The method of claim 19, wherein the plurality of sgRNAs comprises SEQ ID NOs. 1-280.

22. The method of claim 19, wherein the sequencing comprises targeted capture sequencing.

23. The method of claim 19, wherein the mutation comprises a nucleotide insertion.

24. The method of claim 23, wherein the insertion comprises more than one nucleotide base.

25. The method of claim 19, wherein the mutation comprises a nucleotide deletion.

26. The method of claim 25, wherein the deletion comprises more than one nucleotide base.

27. The method of claim 19, wherein the subject is a mammal.

28. The method of claim 27, wherein the mammal is a mouse or a human.

29. A vector comprising an adeno-associated virus (AAV) genome, a U6 promoter gene, an sgRNA sequence, a Glial Fibrillary Acidic Protein (GFAP) promoter gene, and a Cre recombinase gene.

30. The vector of claim 29, wherein the GFAP promoter gene comprises the nucleic acid sequence of SEQ ID NO: 290.

31. A vector comprising the nucleic acid sequence of SEQ ID NO: 289.

32. A kit comprising a vector comprising the nucleic acid sequence of SEQ ID NO: 289, and instructional material for use thereof

33. A kit comprising an adeno-associated virus (AAV) genome, a U6 promoter gene, an sgRNA sequence, a Glial Fibrillary Acidic Protein (GFAP) promoter gene, and a Cre recombinase gene, and instructional material for use thereof.

34. The kit of claim 33, wherein the GFAP promoter gene comprises the nucleic acid sequence of SEQ ID NO: 290.
